(19) United States
(12) Patent Application Publication
(10) Pub. No.: US 2009/0143410 A1
(43) Pub. Date: Jun. 4, 2009

(54) Isoxazolines for Controlling Invertebrate Pests

(76) Inventor: Kanu Maganbhai Patel, Wilmington, DE (US)

Correspondence Address: Birch Linda D;E I Du Pont De Nemours and Company, Legal Patent Records Center, 4417 Lancaster Pike, Wilmington, DE, 19805, US

(21) Appl. No.: 12/083,943

(22) PCT Filed: Dec. 13, 2006
(86) PCT No.: PCT/US2006/047628

§ 371 Date: Apr. 21, 2008

Related U.S. Application Data
(60) Provisional application No. 60/750,136, filed on Dec. 14, 2005.
(60) Provisional application No. 60/753,148, filed on Dec. 22, 2005.
(60) Provisional application No. 60/872,114, filed on Dec. 1, 2006.

(51) Int. Cl. A01N 43/54; C07D 401/04; A01P 7/04

(52) U.S. Cl. 514/256; 544/333

(57) ABSTRACT

Disclosed are compounds of Formula 1, including all geometric and stereoisomers, N-oxides, and salts thereof,

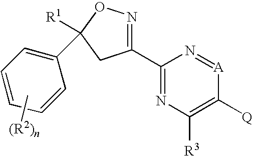

wherein
A is selected from the group consisting of CR³ and N;
each R³ is independently H, halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, —CN, —NO₂ or —CR⁹═NOR¹⁰; or a phenyl or a pyridinyl ring, each ring optionally substituted with one to three substituents independently selected from R⁸;
Q is a 5- or 6-membered saturated or unsaturated heterocyclic ring optionally substituted with one or more substituents independently selected from halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, —CN, —NO₂, —N(R¹¹)R¹², —C(W)N(R¹³)R¹⁴, —C(O)OR¹⁵ and R¹⁶; or
Q is —C(═W)NR⁴R⁵; and
R¹, R², R⁴, R⁵, R⁸, R⁹, R¹⁰, R¹¹, R¹², R¹³, R¹⁴, R¹⁵, R¹⁶, W and n are as defined in the disclosure.

Also disclosed are compositions containing the compounds of Formula 1 and methods for controlling an invertebrate pest comprising contacting the invertebrate pest or its environment with a biologically effective amount of a compound or a composition of the invention.

### FIELD OF THE INVENTION

This invention relates to certain isoxazolines, their N-oxides, salts and compositions suitable for agronomic and nonagronomic uses, including those uses listed below, and methods of their use for controlling invertebrate pests such as arthropods in both agronomic and nonagronomic environments.

### BACKGROUND OF THE INVENTION

The control of invertebrate pests is extremely important in achieving high crop efficiency. Damage by invertebrate pests to growing and stored agronomic crops can cause significant reduction in productivity and thereby result in increased costs to the consumer. The control of invertebrate pests in forestry, greenhouse crops, ornamentals, nursery crops, stored food and fiber products, livestock, household, turf, wood products, and public and animal health is also important. Many products are commercially available for these purposes, but the need continues for new compounds that are more effective, less costly, less toxic, environmentally safer or have different modes of action.

PCT Patent Publication WO 05/085216 discloses isoxazoline derivatives of Formula i as insecticides

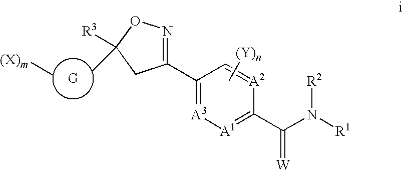

wherein, inter alia, each A¹, A² and A³ is independently C or N; G is a benzene ring; W is O or S; and each X is independently halogen or C₁-C₆ haloalkyl.

### SUMMARY OF THE INVENTION

This invention is directed to compounds of Formula 1 including all geometric and stereoisomers, N-oxides, and salts thereof, and compositions containing them and their use for controlling invertebrate pests:

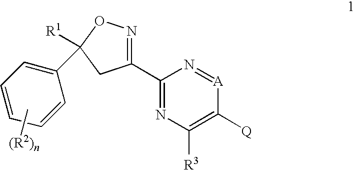

wherein:
A is selected from the group consisting of CR³ and N;
R¹ is C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁶;
each R² is independently H, halogen, C₁-C₆ alkyl, C₁-C₆ haloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkoxycarbonyl, —CN or —NO₂;
each R³ is independently H, halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, —CN, —NO₂, or —CR⁹═NOR¹⁰; or a phenyl ring or a pyridinyl ring, each ring optionally substituted with one to three substituents independently selected from R⁸;
Q is a 5- or 6-membered saturated or unsaturated heterocyclic ring optionally substituted with one or more substituents independently selected from halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, —CN, —NO₂, —N(R¹¹)R¹², —C(W)N(R¹³)R¹⁴, —C(O)OR¹⁵ and R¹⁶; or
Q is —C(═W)NR⁴R⁵;
each R⁴, R¹¹ and R¹³ is independently H, C₁-C₆ alkyl, C₂-C₆ alkenyl, C₂-C₆ alkynyl, C₃-C₆ cycloalkyl, C₄-C₇ alkylcycloalkyl, C₄-C₇ cycloalkylalkyl, C₂-C₇ alkylcarbonyl or C₂-C₇ alkoxycarbonyl;
each R⁵, R¹², R¹⁴ and R¹⁵ is independently H; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁷;
each R⁶ is independently halogen, C₁-C₆ alkyl, C₁-C₆ alkoxy, C₁-C₆ alkylthio, C₁-C₆ alkylsulfinyl, C₁-C₆ alkylsulfonyl, —CN or —NO₂;
each R⁷ is independently halogen, C₁-C₆ alkyl, C₁-C₆ alkoxy, C₁-C₆ alkylthio, C₁-C₆ alkylsulfinyl, C₁-C₆ alkylsulfonyl, C₂-C₇ alkylcarbonyl, C₂-C₇ alkoxycarbonyl, —CN or —NO₂; or Q¹;
each Q¹ is independently a phenyl ring or a 5- or 6-membered saturated or unsaturated heterocyclic ring, each ring optionally substituted with one or more substituents independently selected from halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, —CN, —NO₂, phenyl and pyridinyl;
each R⁸ is independently halogen, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkoxycarbonyl, —CN or —NO₂;
each R⁹ is independently H, NH₂, C₁-C₄ alkyl or C₁-C₄ haloalkyl;
each R¹⁰ is independently H, C₁-C₄ alkyl or C₁-C₄ haloalkyl;
each R¹⁶ is independently a phenyl ring or a pyridinyl ring, each ring optionally substituted with one or more substituents independently selected from R¹⁷;
each R¹⁷ is independently halogen, C₁-C₆ alkyl, C₁-C₆ haloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkylcarbonyl, C₂-C₄ alkoxycarbonyl, C₂-C₇ alkylaminocarbonyl, C₃-C₇ dialkylaminocarbonyl, —OH, —NH₂, —COOH, —CN or —NO₂;
W is O or S; and
n is 1, 2, 3, 4 or 5.

This invention also provides a composition comprising a compound of Formula 1, an N-oxide or a salt thereof, and at least one additional component selected from the group consisting of a surfactant, a solid diluent and a liquid diluent. In one embodiment, this invention also provides a composition for controlling an invertebrate pest comprising a biologically effective amount of a compound of Formula 1, an N-oxide or a salt thereof, and at least one additional component selected from the group consisting of a surfactant, a solid diluent and a liquid diluent, said composition optionally further comprising a biologically effective amount of at least one additional biologically active compound or agent.

This invention further provides a spray composition for controlling an invertebrate pest comprising a biologically effective amount of a compound of Formula 1, an N-oxide or a salt thereof, or the composition described above and a propellant. This invention also provides a bait composition for controlling an invertebrate pest comprising a biologically effective amount of a compound of Formula 1, an N-oxide or a salt thereof, or the composition described in the embodiment above, one or more food materials, optionally an attractant, and optionally a humectant.

This invention further provides a trap device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

This invention also provides a method for controlling an invertebrate pest comprising contacting the invertebrate pest or its environment with a biologically effective amount of a compound of Formula 1, an N-oxide or a salt thereof, (e.g., as a composition described herein). This invention also relates to such method wherein the invertebrate pest or its environment is contacted with a composition comprising a biologically effective amount of a compound of Formula 1, an N-oxide or a salt thereof, and at least one additional component selected from the group consisting of a surfactant, a solid diluent and a liquid diluent, said composition optionally further comprising a biologically effective amount of at least one additional biologically active compound or agent.

This invention also provides a method for protecting a seed from an invertebrate pest comprising contacting the seed with a biologically effective amount of a compound of Formula 1, an N-oxide or a salt thereof. This invention further relates to a treated seed comprising a compound of Formula 1, an N-oxide or a salt thereof, in an amount of from about 0.0001 to 1% by weight of the seed before treatment.

This invention also provides a composition for protecting an animal from an invertebrate parasitic pest comprising a parasiticidally effective amount of a compound of Formula 1, an N-oxide or a salt thereof, and at least one carrier. The present invention further provides the composition described above in a form for oral administration. This invention also provides a method for protecting an animal from an invertebrate parasitic pest comprising administering to the animal a parasiticidally effective amount of a compound of Formula 1, an N-oxide or a salt thereof.

### DETAILS OF THE INVENTION

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in this disclosure, the term “invertebrate pest” includes arthropods, gastropods and nematodes of economic importance as pests. The term “arthropod” includes insects, mites, spiders, scorpions, centipedes, millipedes, pill bugs and symphylans. The term “gastropod” includes snails, slugs and other Stylommatophora. The term “helminths” includes worms in the phyla of Nemathelminthes, Platyhelminthes and Acanthocephalans such as: round worms, heartworms, and phytophagous nematodes (Nematoda), flukes (Trematoda), tape worms (Cestoda) and thorny-headed worms.

In the context of this disclosure “invertebrate pest control” means inhibition of invertebrate pest development (including mortality, feeding reduction, and/or mating disruption), and related expressions are defined analogously.

The term “agronomic” refers to the production of field crops such as for food and fiber and includes the growth of corn, soybeans and other legumes, rice, cereal (e.g., wheat, oats, barley, rye, rice, maize), leafy vegetables (e.g., lettuce, cabbage, and other cole crops), fruiting vegetables (e.g., tomatoes, pepper, eggplant, crucifers and cucurbits), potatoes, sweet potatoes, grapes, cotton, tree fruits (e.g., pome, stone and citrus), small fruit (berries, cherries) and other specialty crops (e.g., canola, sunflower, olives). The term “nonagronomic” refers to other horticultural crops (e.g., greenhouse, nursery or ornamental plants not grown in a field), residential and commercial structures in urban and industrial settings, turf (e.g., sod farm, pasture, golf course, residential lawn, recreational sports field, etc.), wood products, stored product, agro-forestry and vegetation management, public health (human) and animal health (e.g., domesticated animals such as pets, livestock and poultry, undomesticated animals such as wildlife) applications.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkylthio” includes branched or straight-chain alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. “Alkylamino”, “dialkylamino”, and the like, are defined analogously to the above examples. “Cycloalkyl” includes, for example, cyclopropyl, cyclobutyl, cyclopentyl and cyclohexyl. The term “alkylcycloalkyl” denotes alkyl substitution on a cycloalkyl moiety and includes, for example, ethylcyclopropyl, i-propylcyclobutyl, 3-methylcyclopentyl and 4-methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, said alkyl may be partially or fully substituted with halogen atoms which may be the same or different. Examples of “haloalkyl” or “alkyl substituted with halogen” include F₃C—, ClCH₂—, CF₃CH₂— and CF₃CCl₂—. The terms “halocycloalkyl”, “haloalkoxy”, “haloalkylthio”, and the like, are defined analogously to the term “haloalkyl”. Examples of “haloalkoxy” include CF₃O—, CCl₃CH₂O—, HCF₂CH₂CH₂O— and CF₃CH₂O—. Examples of “haloalkylthio” include CCl₃S—, CF₃S—, CCl₃CH₂S— and ClCH₂CH₂CH₂S—. Examples of “haloalkylsulfinyl” include CF₃S(O)—, CCl₃S(O)—, CF₃CH₂S(O)— and CF₃CF₂S(O)—. Examples of “haloalkylsulfonyl” include CF₃S(O)₂—, CCl₃S(O)₂—, CF₃CH₂S(O)₂— and CF₃CF₂S(O)₂—.

“Alkylcarbonyl” denotes a straight-chain or branched alkyl moieties bonded to a C(═O) moiety. Examples of “alkylcarbonyl” include CH₃C(═O)—, CH₃CH₂CH₂C(═O)— and (CH₃)₂CHC(═O)—. Examples of “alkoxycarbonyl” include CH₃C(═O)—, CH₃CH₂C(═O), CH₃CH₂CH₂C(═O)—, (CH₃)₂CHOC(═O)— and the different butoxy- or pentoxycarbonyl isomers.

The total number of carbon atoms in a substituent group is indicated by the “Cᵢ-Cⱼ” prefix where i and j are numbers from 1 to 7. For example, C₁-C₄ alkylsulfonyl designates methylsulfonyl through butylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

When a compound is substituted with a substituent bearing a subscript that indicates the number of said substituents can exceed 1, said substituents (when they exceed 1) are independently selected from the group of defined substituents, e.g., (R²)ₙ, n is 1, 2, 3, 4 or 5. When a group contains a substituent which can be hydrogen, for example R², then when this substituent is taken as hydrogen, it is recognized that this is equivalent to said group being unsubstituted.

“Aromatic” indicates that each of the ring atoms is essentially in the same plane and has a p-orbital perpendicular to the ring plane, and in which (4n+2) π electrons, where n is a positive integer, are associated with the ring to comply with Hückel's rule.

The terms “heterocyclic ring” or “heterocycle” denote a ring in which at least one atom forming the ring backbone is not carbon, e.g., nitrogen, oxygen or sulfur. Typically a heterocyclic ring contains no more than 4 nitrogens, no more than 2 oxygens and no more than 2 sulfurs. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring”, “aromatic heterocyclic ring”. Unless otherwise indicated, heterocyclic rings and ring systems can be attached through any available carbon or nitrogen by replacement of a hydrogen on said carbon or nitrogen.

The term “optionally substituted” in connection with the heterocyclic rings refers to groups which are unsubstituted or have at least one non-hydrogen substituent that does not extinguish the biological activity possessed by the unsubstituted analog. As used herein, the following definitions shall apply unless otherwise indicated. The term “optionally substituted” is used interchangeably with the phrase “substituted or unsubstituted” or with the term “(un)substituted.” Unless otherwise indicated, an optionally substituted group may have a substituent at each substitutable position of the group, and each substitution is independent of the other.

When Q is a 5- or 6-membered nitrogen-containing heterocyclic ring, it may be attached to the remainder of Formula 1 though any available carbon or nitrogen ring atom, unless otherwise described. Similarly, when Q¹ is a 5- or 6-membered nitrogen-containing heterocyclic ring, it may be attached through any available carbon or nitrogen ring atom, unless otherwise described.

As noted above, Q¹ can be (among others) phenyl optionally substituted with one or more substituents selected from a group of substituents as defined in the Summary of Invention. An example of phenyl optionally substituted with one to five substituents is the ring illustrated as U-1 in Exhibit 1, wherein R^{v} is selected from a group of substituents as defined in the Summary of the Invention for Q¹ and r is an integer from 0 to 5.

As noted above, Q or Q¹ can be (among others) a 5- or 6-membered heterocyclic ring, which may be saturated or unsaturated, optionally substituted with one or more substituents selected from a group of substituents as defined in the Summary of Invention. Examples of a 5- or 6-membered unsaturated aromatic heterocyclic ring optionally substituted with from one or more substituents include the rings U-2 through U-61 illustrated in Exhibit 1 wherein R^{v} is any substituent as defined in the Summary of the Invention for Q or Q and r is an integer from 0 to 4, limited by the number of available positions on each U group. As U-29, U-30, U-36, U-37, U-38, U-39, U-40, U-41, U-42 and U-43 have only one available position, for these U groups r is limited to the integers 0 or 1, and r being 0 means that the U group is unsubstituted and a hydrogen is present at the position indicated by (R^{v})ᵣ.

### Exhibit 1

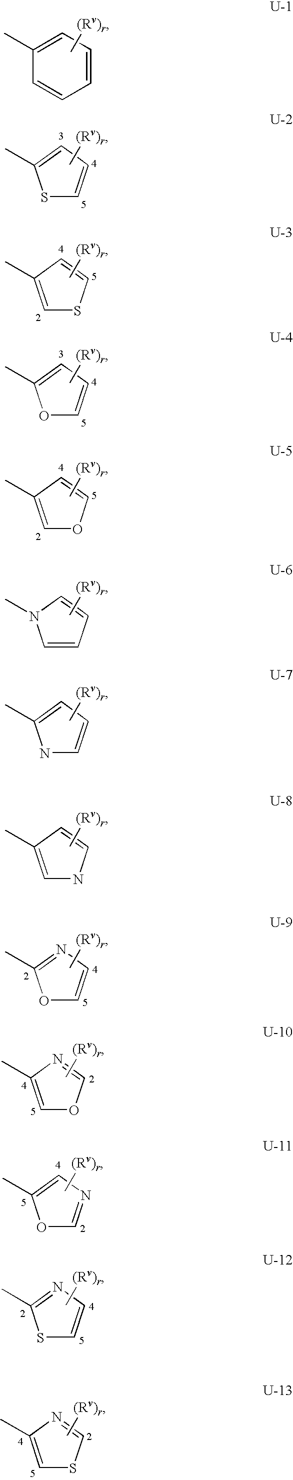

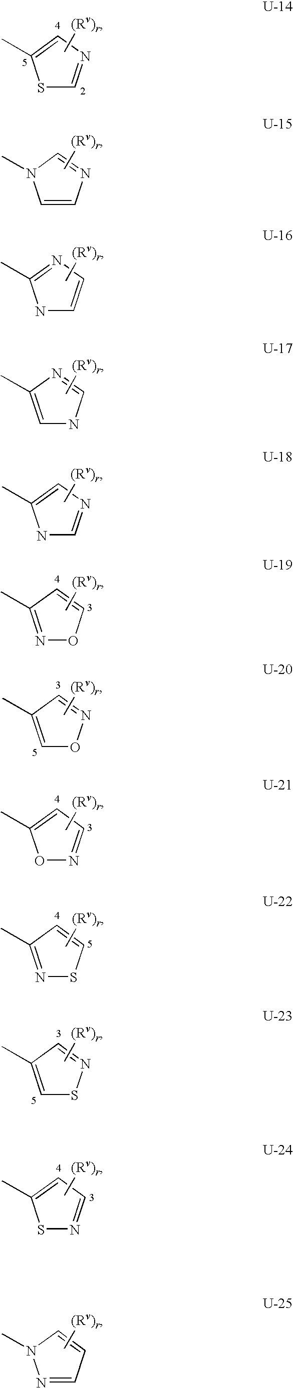

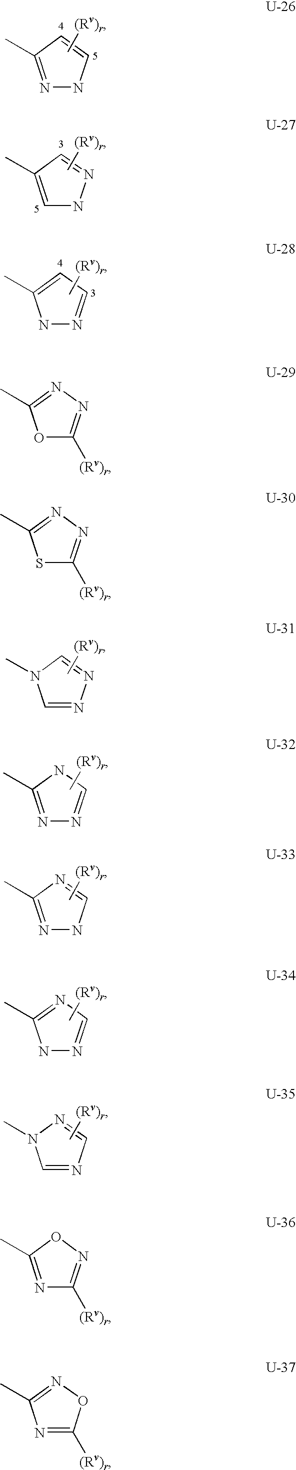

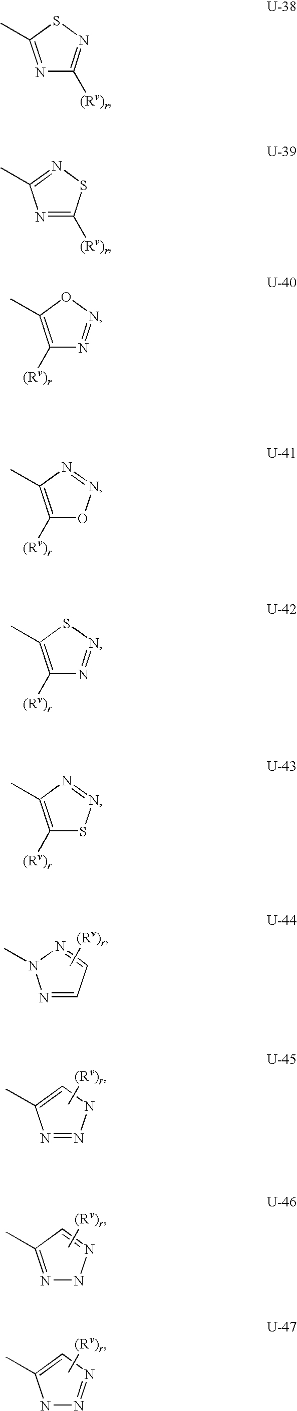

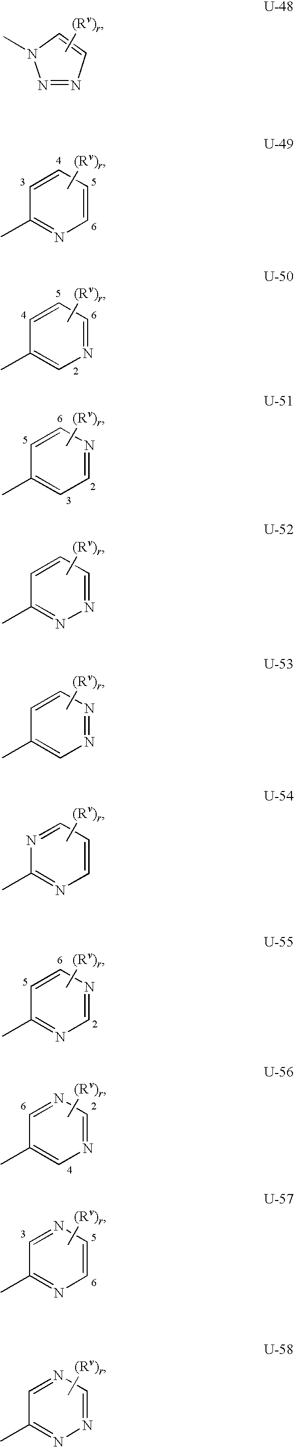

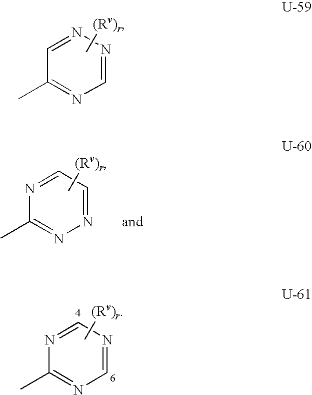

Although R^{v} groups are shown in the structures U-1 through U-61, it is noted that they do not need to be present since they are optional substituents. Note that when R^{v} is H and attached to an atom, this is the same as if said atom is unsubstituted. The nitrogen atoms that require substitution to fill their valence are substituted with H or R^{v}. Note that when the attachment point between (R^{v})ᵣ and the U group is illustrated as floating, (R^{v})ᵣ can be attached to any available carbon atom or nitrogen atom of the U group. Note that when the attachment point on the U group is illustrated as floating, the U group can be attached to the remainder of Formula 1 through any available carbon or nitrogen of the U group by replacement of a hydrogen atom. Note that some U groups can only be substituted with less than four R^{v} groups (e.g., U-2 through U-5, U-7 through U-48, and U-52 through U-61).

Note that when Q or Q¹ is a 5- or 6-membered saturated or unsaturated non-aromatic heterocyclic ring optionally substituted with one or more substituents selected from the group of substituents as defined in the Summary of Invention for Q or Q¹, one or two carbon ring members of the heterocyclic ring can optionally be in the oxidized form of a carbonyl moiety.

Examples of a 5- or 6-membered saturated or non-aromatic unsaturated heterocyclic ring include the rings G-1 through G-35 as illustrated in Exhibit 2. Note that when the attachment point on the G group is illustrated as floating, the G group can be attached to the remainder of Formula 1 through any available carbon or nitrogen of the G group by replacement of a hydrogen atom. The optional substituents corresponding to R^{v} can be attached to any available carbon or nitrogen by replacing a hydrogen atom. For these G rings, r is typically an integer from 0 to 5, limited by the number available positions on each G group.

Note that when Q or Q¹ comprises a ring selected from G-28 through G-35, G² is selected from O, S or N. Note that when G² is N, the nitrogen atom can complete its valence by substitution with either H or the substituents as defined in the Summary of Invention for Q or Q¹.

### Exhibit 2

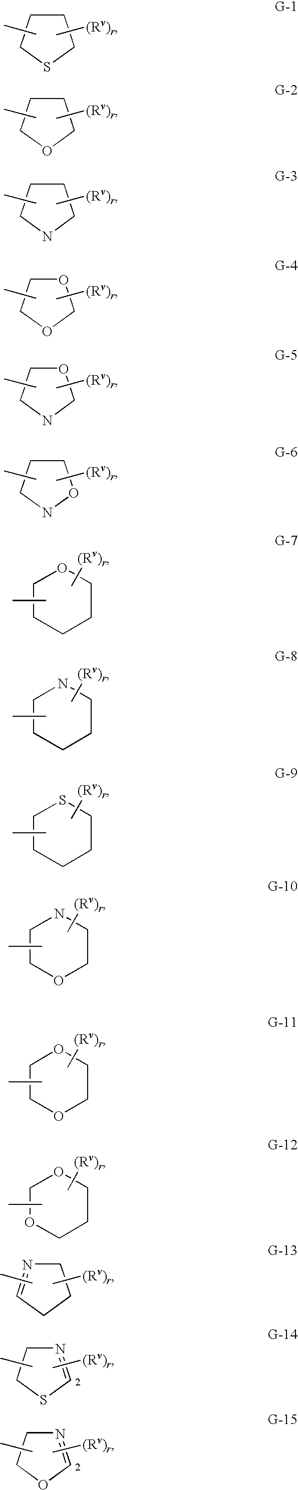

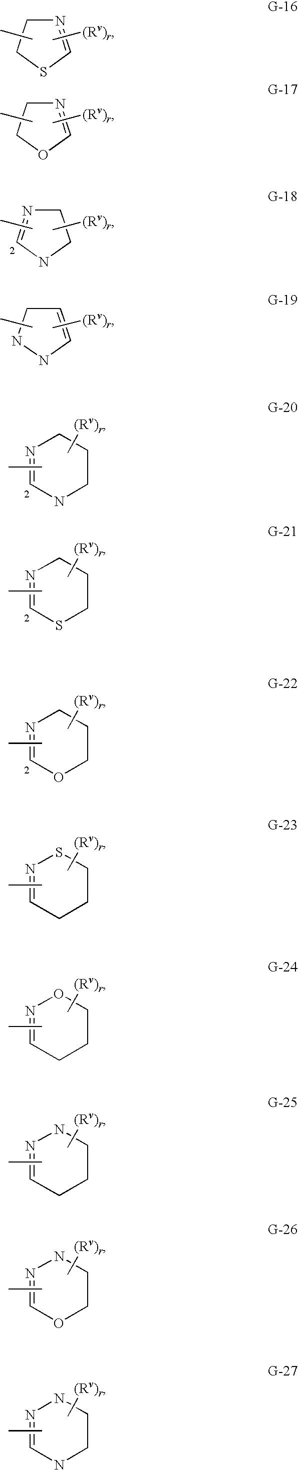

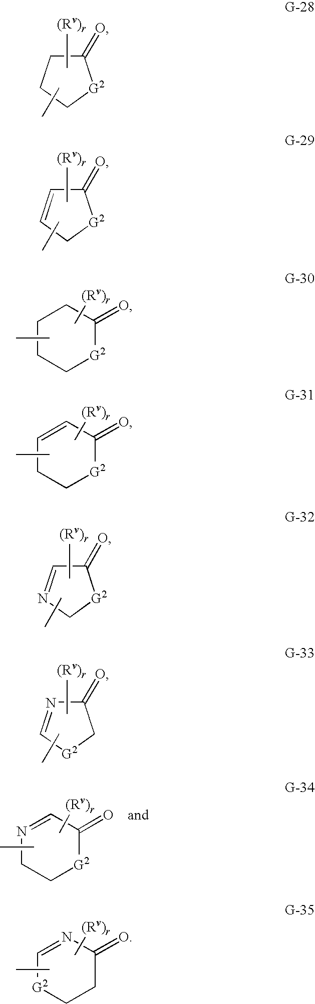

A wide variety of synthetic methods are known in the art to enable preparation of aromatic and nonaromatic heterocyclic rings; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

One skilled in the art will appreciate that not all nitrogen containing heterocyclic rings can form N-oxides since the nitrogen requires an available lone pair for oxidation to the oxide; one skilled in the art will recognize those nitrogen containing heterocyclic rings which can form N-oxides. One skilled in the art will also recognize that tertiary amines can form N-oxides. Synthetic methods for the preparation of N-oxides of heterocyclic rings and tertiary amines are very well known by one skilled in the art including the oxidation of heterocyclic rings and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of undesired vegetation (i.e. are agriculturally suitable). The salts of the compounds of the invention include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. The salts of the compounds of the invention also include those formed with organic bases (e.g., pyridine, ammonia, or triethylamine) or inorganic bases (e.g., hydrides, hydroxides, or carbonates of sodium, potassium, lithium, calcium, magnesium or barium) when the compound contains an acidic moiety such as when R⁴ is alkylcarbonyl and R⁵ is H. Accordingly, the present invention comprises compounds selected from Formula 1, N-oxides and agriculturally suitable salts thereof.

Embodiments of the present invention as described in the Summary of the Invention include:
Embodiment 1. A compound of Formula 1 wherein R¹ is C₁-C₃ alkyl optionally substituted with one or more substituents independently selected from R⁶.
Embodiment 2. A compound of Embodiment 1 wherein R¹ is C₁-C₃ alkyl independently substituted with halogen.
Embodiment 3. A compound of Embodiment 2 wherein R¹ is CF₃.
Embodiment 4. A compound of Formula 1 wherein each R⁶ is independently halogen.
Embodiment 5. A compound of Formula 1 wherein each R² is independently H, halogen, C₁-C₆ haloalkyl, C₁-C₆ haloalkoxy or —CN.
Embodiment 6. A compound of Embodiment 5 wherein each R² is independently H, halogen, CF₃, OCF₃ or —CN.
Embodiment 7. A compound of Formula 1 wherein each R³ is independently H, halogen, C₁-C₆ alkyl, C₁-C₆ haloalkyl, C₃-C₆ cycloalkyl, C₁-C₆ alkoxy, C₁-C₆ haloalkoxy, —CN, —NO₂ or —CR⁹═NOR¹⁰; or a phenyl ring or a pyridinyl ring, each ring optionally substituted with one to three substituents independently selected from R⁸.
Embodiment 8. A compound of Formula 1 wherein each R⁹ is independently C₁-C₄ alkyl.
Embodiment 9. A compound of Formula 1 wherein each R¹⁰ is independently H
Embodiment 10. A compound of Embodiment 7 wherein each R³ is independently H, C₁-C₄ alkyl, C₁-C₄ haloalkyl, cyclopropyl, C₁-C₄ alkoxy, —CN or —NO₂; or a phenyl ring optionally substituted with one to three substituents independently selected from R⁸.
Embodiment 11. A compound of Embodiment 10 wherein each R³ is independently H, C₁-C₄ alkyl or C₁-C₄ haloalkyl; or a phenyl ring optionally substituted with one to three substituents independently selected from R⁸.
Embodiment 12. A compound of Formula 1 wherein each R⁸ is independently halogen, C₁-C₆ alkoxy, C₁-C₆ haloalkoxy, C₂-C₄ alkoxycarbonyl, —CN or —NO₂.
Embodiment 13. A compound of Embodiment 12 wherein each R⁸ is independently halogen.
Embodiment 14. A compound of Formula 1 wherein Q is a pyridinyl ring, a pyrimidinyl ring, a triazinyl ring, a pyrazolyl ring, a triazolyl ring, a tetrazolyl ring, an imidazolyl ring, an oxazolyl ring, an isoxazolyl ring, a thiazolyl ring or an isothiazolyl ring, each ring optionally substituted with one or more substituents independently selected from the group consisting of halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, —CN, —NO₂, —N(R¹¹)R¹², —C(W)N(R¹³)R¹⁴, —C(O)OR¹⁵ and R¹⁶.
Embodiment 15. A compound of Embodiment 14 wherein Q is a pyrazolyl ring, a triazolyl ring, a tetrazolyl ring or an imidazolyl ring, each ring attached to the remainder of Formula 1 through nitrogen and optionally substituted with one or more substituents independently selected from the group consisting of halogen, C₁-C₄ alkyl, C₁-C₄ haloalkyl, C₃-C₆ cycloalkyl, C₃-C₆ halocycloalkyl, C₁-C₄ alkoxy, C₁-C₄ haloalkoxy, —CN, —NO₂, —N(R¹¹)R¹², —C(W)N(R¹³)R¹⁴, —C(O)OR¹⁵ and R¹⁶.
Embodiment 16. A compound of Embodiment 15 wherein Q is a pyrazolyl ring, a triazolyl ring, a tetrazolyl ring or an imidazolyl ring, each ring attached to the remainder of Formula 1 through nitrogen and optionally substituted with one or more substituents independently selected from the group consisting of halogen, C₁-C₄ alkyl, C₁-C₄ haloalkyl, C₁-C₄ alkoxy, C₁-C₄ haloalkoxy, —CN and —NH₂.
Embodiment 17. A compound of Embodiment 16 wherein Q is a pyrazolyl ring, a triazolyl ring, a tetrazolyl ring or an imidazolyl ring, each ring attached to the remainder of Formula 1 through nitrogen.
Embodiment 18. A compound of Embodiment 17 wherein Q is a triazolyl ring attached to the remainder of Formula 1 through nitrogen.
Embodiment 19. A compound of Formula 1 wherein Q is —C(═W)NR⁴R⁵.
Embodiment 20. A compound of Formula 1 wherein each R⁴, R¹¹ and R¹³ is independently H, C₁-C₆ alkyl, C₂-C₇ alkylcarbonyl or C₂-C₇ alkoxycarbonyl.
Embodiment 21. A compound of Embodiment 20 wherein each R⁴, R¹¹ and R¹³ is independently H.
Embodiment 22. A compound of Formula 1 wherein each R⁵, R¹², R¹⁴ and R¹⁵ is independently H; or C₁-C₄ alkyl, C₂-C₄ alkenyl, C₂-C₄ alkynyl, C₃-C₄ cycloalkyl, C₄-C₇ alkylcycloalkyl or C₄-C₇ cycloalkylalkyl, each optionally substituted with one or more substituents independently selected from R⁷.
Embodiment 23. A compound of Embodiment 22 wherein each R⁵, R¹², R¹⁴ and R¹⁵ is independently H; or C₁-C₄ alkyl optionally substituted with one of more substituents independently selected from R⁷.
Embodiment 24. A compound of Formula 1 wherein each R⁷ is independently halogen, C₁-C₄ alkyl, C₁-C₄ alkoxy, C₁-C₄ alkylthio, C₁-C₄ alkylsulfinyl, C₁-C₄ alkylsulfonyl, C₂-C₄ alkylcarbonyl, C₂-C₄ alkoxycarbonyl, —CN, —NO₂ or Q¹.
Embodiment 25. A compound of Formula 1 wherein each Q¹ is independently a phenyl ring, a pyridinyl ring or a thiazolyl ring, each ring optionally substituted with one or more substituents independently selected from halogen, C₁-C₃ alkyl, C₁-C₃ haloalkyl, —CN, phenyl and pyridinyl.
Embodiment 26. A compound of Embodiment 25 wherein Q¹ is a phenyl ring, a pyridinyl ring or a thiazolyl ring.
Embodiment 27. A compound of Formula 1 wherein R⁵ is C₁-C₄ alkyl optionally substituted with one or more substituents independently selected from R⁷.
Embodiment 28. A compound of Embodiment 27 wherein R⁵ is C₁-C₄ alkyl optionally substituted with one Q¹ and optionally substituted with one or more fluorine.
Embodiment 29. A compound of Formula 1 wherein each R⁷ is independently halogen or Q¹.
Embodiment 30. A compound of Embodiment 29 wherein each R⁷ is independently F or Q¹.
Embodiment 31. A compound of Embodiment 30 wherein each R⁷ is F.
Embodiment 32. A compound of Embodiment 27 wherein R⁵ is CH₂CF₃.
Embodiment 33. A compound of Embodiment 27 wherein R⁵ is CH₂-2-pyridinyl.
Embodiment 34. A compound of Formula 1 wherein W is O.
Embodiment 35. A compound of Formula 1 wherein n is 1 or 2.
Embodiment 36. A compound of Formula 1 wherein A is CR³.
Embodiment 37. A compound of Formula 1 wherein A is N.

Embodiments of this invention, including Embodiments 1-37 above as well as any other embodiments described herein, can be combined in any manner, and the descriptions of variables in the embodiments pertain not only to the compounds of Formula 1 but also to the starting compounds and intermediate compounds. In addition, embodiments of this invention, including Embodiments 1-37 above as well as any other embodiments described herein, and any combination thereof, pertain to the compositions and methods of the present invention.

Combinations of Embodiments 1-37 are illustrated by:

Embodiment A. A compound of Formula 1 wherein
R¹ is C₁-C₃ alkyl optionally substituted with one or more substituents independently selected from R⁶;
each R² is independently H, halogen, C₁-C₆ haloalkyl, C₁-C₆ haloalkoxy or —CN;
each R³ is independently H, halogen, C₁-C₆ alkyl, C₁-C₆ haloalkyl, C₃-C₆ cycloalkyl, C₁-C₆ alkoxy, C₁-C₆ haloalkoxy, —CN, —NO₂ or —CR⁹═NOR¹⁰; or a phenyl ring or a pyridinyl ring, each ring optionally substituted with one to three substituents independently selected from R⁸;
Q is a pyridinyl ring, a pyrimidinyl ring, a triazinyl ring, a pyrazolyl ring, a triazolyl ring, a tetrazolyl ring, an imidazolyl ring, an oxazolyl ring, an isoxazolyl ring, a thiazolyl ring or an isothiazolyl ring, each ring optionally substituted with one or more substituents independently selected from the group consisting of halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, —CN, —NO₂, —N(R¹¹)R¹², —C(W)N(R¹³)R¹⁴, —C(O)OR¹⁵ and R¹⁶; or
Q is C(═W)NR⁴R⁵;
each R⁴, R¹¹ and R¹³ is independently H, C₁-C₆ alkyl, C₂-C₇ alkylcarbonyl or C₂-C₇ alkoxycarbonyl;
each R⁵, R¹², R¹⁴ and R¹⁵ is independently H; or C₁-C₄ alkyl, C₂-C₄ alkenyl, C₂-C₄ alkynyl, C₃-C₄ cycloalkyl, C₄-C₇ alkylcycloalkyl or C₄-C₇ cycloalkylalkyl, each optionally substituted with one or more substituents independently selected from R⁷; and
each R⁷ is independently halogen, C₁-C₄ alkyl, C₁-C₄ alkoxy, C₁-C₄ alkylthio, C₁-C₄ alkylsulfinyl, C₁-C₄ alkylsulfonyl, C₂-C₄ alkylcarbonyl, C₂-C₄ alkoxycarbonyl, —CN, —NO₂ or Q¹.

Embodiment B. A compound of Embodiment A wherein
R¹ is C₁-C₃ alkyl independently substituted with halogen;
each R² is independently H, halogen, CF₃, OCF₃ or —CN;
each R³ is independently H, C₁-C₄ alkyl, C₁-C₄ haloalkyl, cyclopropyl, C₁-C₄ alkoxy, —CN or —NO₂; or a phenyl ring optionally substituted with one to three substituents independently selected from R⁸;
Q is a pyrazolyl ring, a triazolyl ring, a tetrazolyl ring or an imidazolyl ring, each ring attached to the remainder of Formula 1 through nitrogen and optionally substituted with one or more substituents independently selected from the group consisting of halogen, C₁-C₄ alkyl, C₁-C₄ haloalkyl, C₁-C₄ alkoxy, C₁-C₄ haloalkoxy, —CN and NH₂; or
Q is —C(═W)NR⁴R⁵;
R⁴ is H;
R⁵ is C₁-C₄ alkyl optionally substituted with one of more substituents independently selected from R⁷;
each R⁷ is independently halogen or Q¹; and
Q¹ is a phenyl ring, a pyridinyl ring or a thiazolyl ring, each ring optionally substituted with one or more substituents independently selected from the group consisting of halogen, C₁-C₃ alkyl, C₁-C₃ haloalkyl, —CN, phenyl and pyridinyl.

Embodiment C. A compound of Embodiment B wherein
R¹ is CF₃; and
R⁵ is CH₂CF₃ or CH₂-2-pyridinyl.

Specific embodiments include compounds of Formula 1 selected from the group consisting of:
2-[5-(3,5-dichlorophenyl)-4,5-dihydro-5-(trifluoromethyl)-3-isoxazolyl]-N-(2-pyridinylmethyl)-4-(trifluoromethyl)-5-pyrimidinecarboxamide;
2-[5-(3,5-dichlorophenyl)-4,5-dihydro-5-(trifluoromethyl)-3-isoxazolyl]-N-(2,2,2-trifluoroethyl)-4-(trifluoromethyl)-5-pyrimidinecarboxamide;
2-[5-(3,5-dichlorophenyl)-4,5-dihydro-5-(trifluoromethyl)-3-isoxazolyl]-4-methyl-N-(2,2,2-trifluoroethyl)-5-pyrimidinecarboxamide; and
2-[5-(3,5-dichlorophenyl)-4,5-dihydro-5-(trifluoromethyl)-3-isoxazolyl]-4-methyl-N-(2-pyridinylmethyl)-5-pyrimidinecarboxamide.

Further specific embodiments include any combination of the compounds of Formula 1 selected from the group immediately above.

Embodiments of the present invention further include:

Embodiment AA. A compound of Formula 1p, an N-oxide, or a salt thereof,

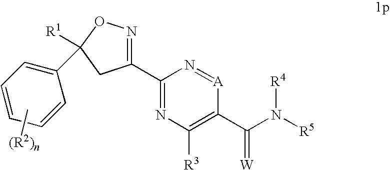

wherein:
A is selected from the group consisting of CR³ and N;
R¹ is C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁶;
each R² is independently H, halogen, C₁-C₆ alkyl, C₁-C₆ haloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkoxycarbonyl, —CN or —NO₂;
each R³ is independently H, halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, —CN or —NO₂; or a phenyl ring or a pyridinyl ring, each ring optionally substituted with one to three substituents independently selected from R⁸;
R⁴ is H, C₁-C₆ alkyl, C₂-C₆ alkenyl, C₂-C₆ alkynyl, C₃-C₆ cycloalkyl, C₄-C₇ alkylcycloalkyl, C₄-C₇ cycloalkylalkyl, C₂-C₇ alkylcarbonyl or C₂-C₇ alkoxycarbonyl;
R⁵ is H; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁷;
each R⁶ is independently halogen, C₁-C₆ alkyl, C₁-C₆ alkoxy, C₁-C₆ alkylthio, C₁-C₆ alkylsulfinyl, C₁-C₆ alkylsulfonyl, —CN or —NO₂;
each R⁷ is independently halogen, C₁-C₆ alkyl, C₁-C₆ alkoxy, C₁-C₆ alkylthio, C₁-C₆ alkylsulfinyl, C₁-C₆ alkylsulfonyl, C₂-C₇ alkylcarbonyl, C₂-C₇ alkoxycarbonyl, —CN or —NO₂; or Q¹;
each Q¹ is independently a phenyl ring or a 5- or 6-membered saturated or unsaturated heterocyclic ring, each ring optionally substituted with one or more substituents independently selected from halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, —CN, —NO₂, phenyl and pyridinyl;
each R⁸ is independently halogen, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkoxycarbonyl, —CN or —NO₂;
W is O or S; and
n is 1, 2, 3, 4 or 5.

Embodiment BB. A compound of Embodiment AA wherein
R¹ is C₁-C₃ alkyl optionally substituted with one or more substituents selected from R⁶;
each R² is independently selected from the group consisting of H, halogen, C₁-C₆ haloalkyl, C₁-C₆ haloalkoxy and —CN;
each R³ is independently selected from the group consisting of H, halogen, C₁-C₆ alkyl, C₁-C₆ haloalkyl, C₃-C₆ cycloalkyl, C₁-C₆ alkoxy, C₁-C₆ haloalkoxy, —CN and NO₂; or a phenyl ring or a pyridinyl ring, each ring optionally substituted with one to three substituents independently selected from R⁸;
R⁴ is H, C₁-C₆ alkyl, C₂-C₇ alkylcarbonyl or C₂-C₇ alkoxycarbonyl;
R⁵ is H; or C₁-C₄ alkyl, C₂-C₄ alkenyl, C₂-C₄ alkynyl, C₃-C₄ cycloalkyl, C₄-C₇ alkylcycloalkyl or C₄-C₇ cycloalkylalkyl, each optionally substituted with one or more substituents selected from R⁷; and
each R⁷ is independently selected from the group consisting of halogen, C₁-C₄ alkyl, C₁-C₄ alkoxy, C₁-C₄ alkylthio, C₁-C₄ alkylsulfinyl, C₁-C₄ alkylsulfonyl, C₂-C₄ alkylcarbonyl, C₂-C₄ alkoxycarbonyl, —CN and —NO₂; or Q¹.

Embodiment CC. A compound of Embodiment BB wherein
R¹ is C₁-C₃ alkyl substituted with halogen;
each R² is independently selected from the group consisting of H, CF₃, OCF₃, halogen and —CN;
each R³ is independently selected from the group consisting of H, C₁-C₄ alkyl, C₁-C₄ haloalkyl, cyclopropyl, C₁-C₄ alkoxy, —CN and —NO₂; or phenyl optionally substituted with one to three substituents independently selected from R⁸;
R⁴ is H;
R⁵ is C₁-C₄ alkyl optionally substituted with one of more substituents selected from R⁷; and
Q¹ is phenyl, pyridinyl or thiazolyl, each optionally substituted with one or more substituents independently selected from halogen, C₁-C₃ alkyl, C₁-C₃ haloalkyl, —CN, phenyl and pyridinyl.

Embodiment DD. A compound of Embodiment CC wherein
R¹ is CF₃; and
R⁵ is CH₂CF₃ or CH₂-2-pyridinyl.

Specific embodiments include compounds of Formula 1p selected from the group consisting of:
2-[5-(3,5-dichlorophenyl)-4,5-dihydro-5-(trifluoromethyl)-3-isoxazolyl]-4-methyl-N-(2,2,2-trifluoroethyl)-5-pyrimidinecarboxamide; and
2-[5-(3,5-dichlorophenyl)-4,5-dihydro-5-(trifluoromethyl)-3-isoxazolyl]-4-methyl-N-(2-pyridinylmethyl)-5-pyrimidinecarboxamide.

Also noteworthy as embodiments of the present invention are compositions comprising a compound of any of the preceding Embodiments, as well as any other embodiments described herein, and any combinations thereof, and at least one additional component selected from the group consisting of a surfactant, a solid diluent and a liquid diluent, said compositions optionally further comprising at least one additional biologically active compound or agent.

Further noteworthy as embodiments of the present invention are compositions for controlling an invertebrate pest comprising a biologically effective amount of a compound of any of the preceding Embodiments, and at least one additional component selected from the group consisting of a surfactant, a solid diluent and a liquid diluent, said composition optionally further comprising a biologically effective amount of at least one additional biologically active compound or agent. Embodiments of the invention further include methods for controlling an invertebrate pest comprising contacting the invertebrate pest or its environment with a biologically effective amount of a compound of any of the preceding Embodiments (e.g., as a composition described herein).

Embodiments of the invention also include a composition comprising a compound of any of the preceding Embodiments, in the form of a soil drench liquid formulation. Embodiments of the invention further include methods for controlling an invertebrate pest comprising contacting the soil with a liquid composition as a soil drench comprising a biologically effective amount of a compound of any of the preceding Embodiments.

Embodiments of the invention also include a spray composition for controlling an invertebrate pest comprising a biologically effective amount of a compound of any of the preceding Embodiments and a propellant. Embodiments of the invention further include a bait composition for controlling an invertebrate pest comprising a biologically effective amount of a compound of any of the preceding Embodiments, one or more food materials, optionally an attractant, and optionally a humectant. Embodiments of the invention also include a device for controlling an invertebrate pest comprising said bait composition and a housing adapted to receive said bait composition, wherein the housing has at least one opening sized to permit the invertebrate pest to pass through the opening so the invertebrate pest can gain access to said bait composition from a location outside the housing, and wherein the housing is further adapted to be placed in or near a locus of potential or known activity for the invertebrate pest.

Embodiments of the invention also include a method for protecting a seed from an invertebrate pest comprising contacting the seed with a biologically effective amount of a compound of any of the preceding Embodiments. Embodiments of the invention further include a treated seed comprising a compound of any of the preceding Embodiments in an amount of from about 0.0001 to 1% by weight of the seed before treatment.

Embodiments of the invention also include a composition for protecting an animal from an invertebrate parasitic pest comprising a parasiticidally effective amount of a compound of any of the preceding Embodiments and at least one carrier. Embodiments of the invention also include a composition comprising a compound of any of the preceding Embodiments in a form for oral administration. Embodiments of the invention further include a method for protecting an animal from an invertebrate parasitic pest comprising administering to the animal a parasiticidally effective amount of a compound of any of the preceding Embodiments.

Compounds of Formula 1 can be prepared by one or more of the following methods and variations as described in Schemes 1-11. The definitions of R¹, R², R³, R⁴, R⁵, A, Q, n and W in the compounds of Formulae 1-18 below are as defined above in the Summary of the Invention. Formula 1a is a subset of Formula 1, Formula 2a is a subset of Formula 2, Formula 3a is a subset of Formula 3, Formulae 5a and 5b are subsets of Formula 5, and Formula 15a is a subset of Formula 15.

Compounds of Formula 1 can be prepared by the cycloaddition of styrenes of Formula 2 with nitrile oxides derived from oximes of Formula 3 as outlined in Scheme 1. The reaction typically proceeds through the intermediacy of an in situ generated hydroxamyl chloride. In a typical procedure a chlorinating reagent such as sodium hypochlorite, N-chlorosuccinimide, or chloramine-T is combined with the oxime in the presence of the styrene. Depending on the conditions, amine bases such as pyridine or triethylamine may be necessary. The reaction can be run in a wide variety of solvents including tetrahydrofuran, diethyl ether, methylene chloride, dioxane, and toluene with optimum temperatures ranging from room temperature to the reflux temperature of the solvent. General procedures for cycloaddition of nitrile oxides with olefins are well documented in the chemical literature. For relevant references see Lee, Synthesis, 1982, 6, 508-509 and Kanemasa et al., Tetrahedron, 2000, 56, 1057-1064 as well as references cited within. The method of Scheme 1 is illustrated in Example 2, Step E and Example 3, Step D.

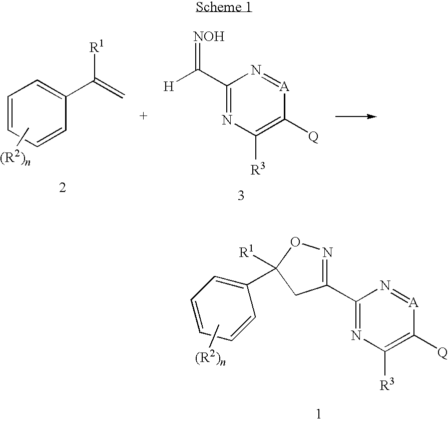

Oximes of Formula 3 can be prepared from contacting the corresponding aldehydes with hydroxylamine according to known methods as shown in Scheme 2.

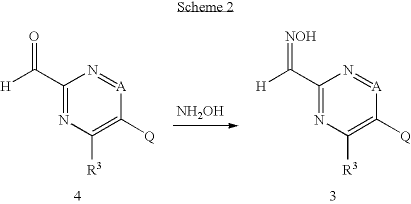

Alternatively, oximes of Formula 3 can be prepared directly from acetals of Formula 5 and an excess of hydroxylamine hydrochloride as shown in the Scheme 3. This reaction can be run in a wide variety of polar solvents including ethanol, methanol or water with optimum temperatures ranging from room temperature to the reflux temperature of the solvent. The method of Scheme 3 is illustrated in Example 2, Step D and Example 3, Step C.

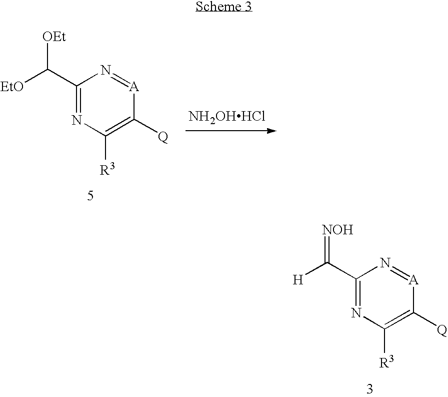

Compounds of Formula 5 can be prepared by following literature methods, see Schaefer F. C., Peters G. A., J. Org. Chem., 1961, 26, 412. For example, compounds of Formula 5a, (i.e. Formula 5 wherein A is CR³) wherein each R³ can be either the same or different, can be prepared as shown in Scheme 4.

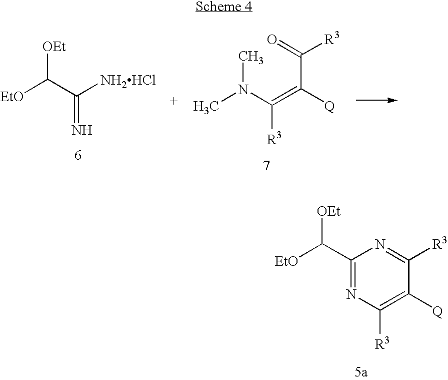

In the method of Scheme 4, compounds of Formula 5a are prepared by the reaction of compounds of Formulae 6 and 7; typically a base such as triethylamine or sodium ethoxide is needed. The reaction can be run in a wide variety of solvents including tetrahydrofuran, u toluene or ethanol, with optimum temperatures ranging from room temperature to the reflux temperature of the solvent. The method of Scheme 4 is illustrated in Example 3, Step B.

Compounds of Formula 7 can be prepared by modification of known procedures, see Menozzi, G., J. Heterocyclic Chem., 1987, 24, 1669. For a specific example, compounds of the Formula 7, wherein Q is a triazolyl ring can be prepared by known methods, see Abdel-Megid, M., et al.; J. Heterocyclic Chem., 2002, 39, 105-108 and German patent application DE 3144670. An example of preparing a compound of Formula 7 is illustrated in Example 3, Step A.

An especially useful group of styrenes for the synthesis of compounds of Formula 1 are represented by Formula 2a (i.e. Formula 2 wherein R¹ is CF₃) as shown in Scheme 5. These intermediates can be prepared by the palladium-catalyzed coupling of commercially available 2-bromo-3,3,3-trifluoropropene (Formula 8) with aryl boronic acids of Formula 9. A typical procedure for the method of Scheme 5 is described in Example 1, Step C.

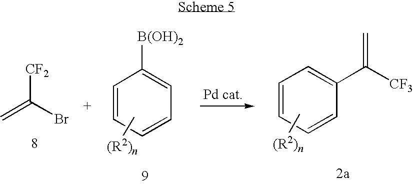

Compounds of Formula 1a (i.e. Formula 1 wherein Q is —C(═W)NR⁴R⁵) wherein W is can be prepared from compounds of Formula 10 as shown in Scheme 6. This method first involves hydrolysis of the ester of Formula 10 with a base such as sodium or lithium hydroxide and conversion of the resulting acid of Formula 11 to the acid chloride of Formula 12 by known methods such as reaction with oxalyl chloride or thionyl chloride. Compounds of Formula 1a wherein W is O are then prepared by treatment of the acid chlorides of Formula 12 with amines of Formula 13. A base such as triethylamine or pyridine may be needed. The reaction can be run in a wide variety of solvents including tetrahydrofuran, diethyl ether, methylene chloride, dioxane and toluene, with optimum temperatures ranging from room temperature to the reflux temperature of the solvent. Compounds of Formula 1a wherein W is S can be then prepared by treatment of the corresponding amide compounds of Formula 1a wherein W is O with thio transfer reagents, such as P₂S₅ (see for example, E. Klingsberg et al., J. Am. Chem. Soc. 1951, 72, 4988; E. C. Taylor Jr. et al., J. Am. Chem. Soc. 1953, 75, 1904; R. Crossley et al., J. Chem. Soc. Perkin Trans. 11976, 977) or Lawesson's reagent (2,5-bis(4-methoxyphenyl)-1,3-dithia-2,4-diphosphetane-2,4-disulfide; see, for example, S. Prabhakar et al. Synthesis, 1984, 829). The method of Scheme 6 is illustrated in Example 1, Steps E and F.

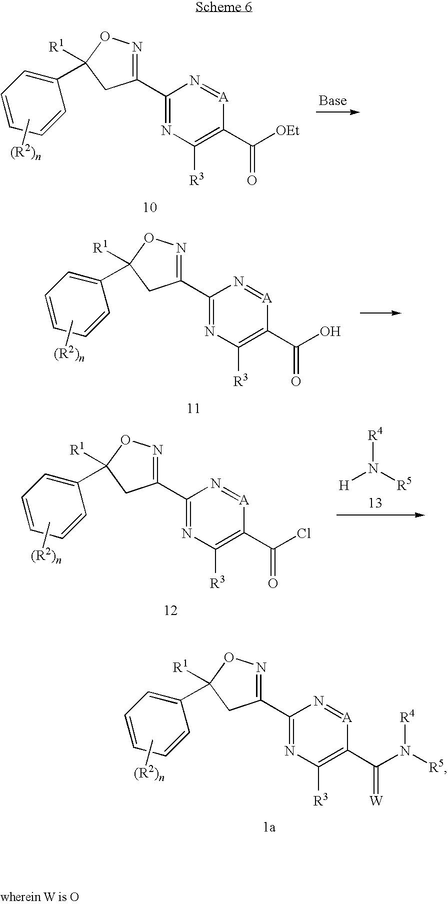

As outlined in Scheme 7, compounds of Formula 10 can be prepared by the cycloaddition of styrenes of Formula 2 with oximes of Formula 14 using methods analogous to those already described for Scheme 1. The method of Scheme 7 is illustrated in Example 1, Step D.

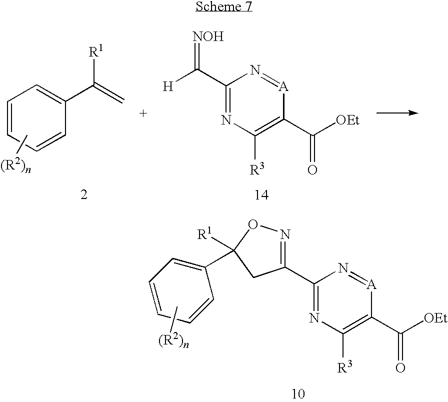

As shown in Scheme 8, condensation of the acetals of Formula 15 with excess hydroxylamine hydrochloride provides the oximes of Formula 14. Conditions for the method of Scheme 8 are analogous to those described for the method of Scheme 3. The method of Scheme 8 is illustrated in Example 1, Step B.

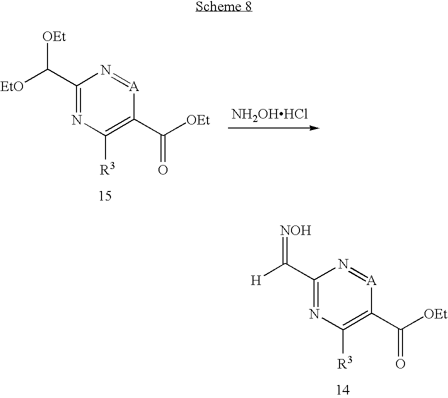

Compounds of the Formula 15 can be prepared using the conditions similar to what is described for the preparation of compounds of the Formula 5 in Scheme 4. As shown in Scheme 9, compounds of Formula 15a (i.e. Formula 15 wherein A is CR³) wherein each R³ can be either the same or different, are prepared by the reaction of compounds of Formulae 6 and 16. The method of Scheme 9 is illustrated in Example 1, Step A.

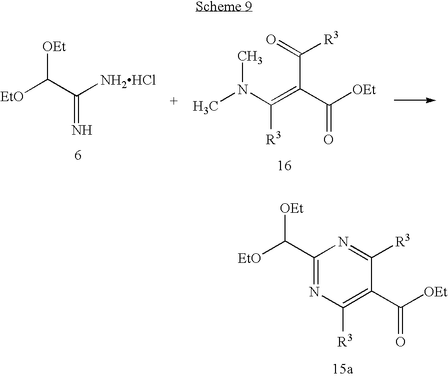

Alternatively, compounds of Formula 1a wherein W is O can also be prepared by the method shown in Scheme 10, which is a subset of the method of Scheme 1. The method of Scheme 10 is illustrated in Example 2, Step E.

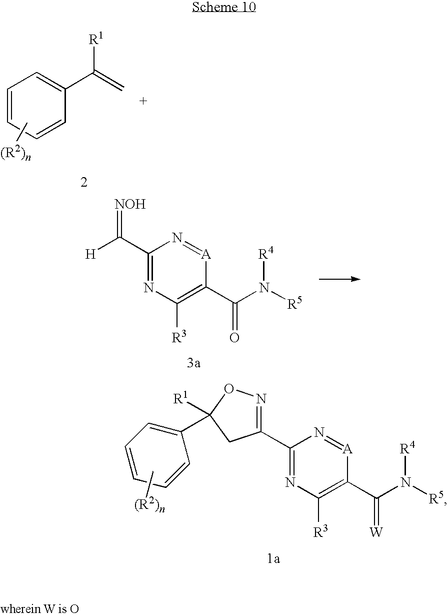

Compounds of Formula 3a (i.e. Formula 3 wherein Q is —C(═W)NR⁴R⁵ and W is O) can be prepared from compounds of Formula 15 as shown in Scheme 11, which is analogous to the method of Scheme 6 followed by the method of Scheme 3. The acid chloride of Formula 18 can be prepared by hydrolysis of the ester of Formula 15 with a base such as sodium hydroxide, followed by conversion of the resulting acid of Formula 17 to the corresponding acid chloride of Formula 18. Treatment of the acid chlorides 18 with amines of Formula 13 provides compounds of Formula 5b (i.e. 5 wherein Q is —C(═W)NR⁴R⁵ and W is O). Compounds of Formula 3a can then be prepared from compounds of Formula 5b by the method of Scheme 3. The method of Scheme 11 is illustrated in Example 2, Steps A, B, C and D.

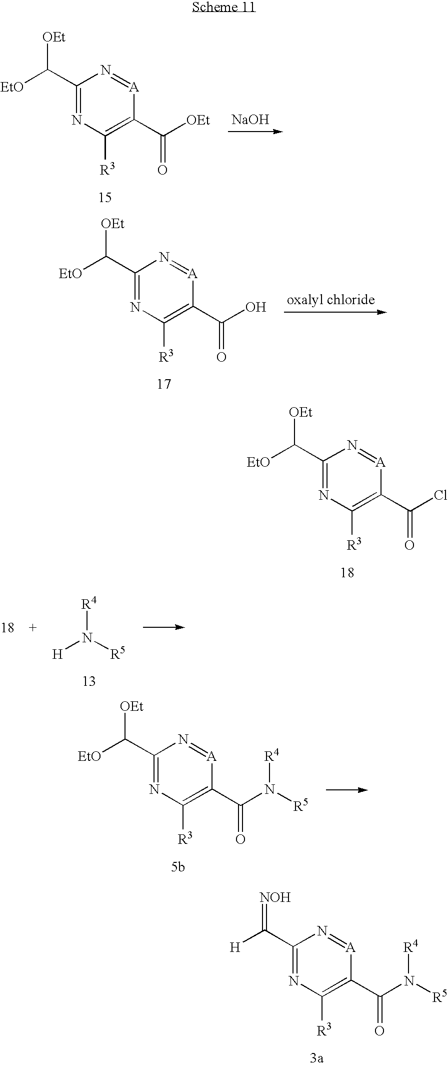

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. ¹H NMR spectra are reported in ppm downfield from tetramethylsilane; “s” means singlet, “d” means doublet, “t” means triplet, “q” means quartet, “m” means multiplet and “br s” means broad singlet.

### EXAMPLE 1

### Preparation of 2-[5-(3,5-dichlorophenyl)-4,5-dihydro-5-(trifluoromethyl)-3-isoxazolyl]-4-methyl-N-(2,2,2-trifluoroethyl)-5-pyrimidinecarboxamide

### Step A: Preparation of Ethyl 2-(diethoxymethyl)-4-methyl-5-pyrimidinecarboxylate

A solution of ethyl 2-[(dimethylamino)methylene]-3-oxobutanoate (see J. Heterocyclic Chem. 1987, 24, 1669 for preparation) (1.85 g, 0.01 mol) in ethanol (10 mL) was added to a mixture of 2,2-diethoxyethanimidamide monohydrochloride (also known as diethoxyacetamidine hydrochloride) (see J. Org Chem. 1961, 26, 412 for preparation) (1.45 g, 0.01 mol) and sodium ethoxide (3.15 mL of 21% in ethanol). The reaction mixture was heated at reflux for 18 h. The resulting mixture was concentrated under reduced pressure, and the residue was suspended in water, followed by extraction with dichloromethane (2×50 mL). The organic extracts were dried (MgSO₄) and concentrated under reduced pressure to afford the title compound as a yellow oil (2.3 g).

¹H NMR (CDCl₃): δ 9.2 (s, 1H), 5.6 (s, 1H), 4.4 (q, 2H), 3.8 (q, 2H), 3.6 (q, 2H), 2.68 (s, 3H), 1.29 (q, 2H), 1.25 (m, 6H).

### Step B: Preparation of Ethyl 2-[(hydroxyimino)methyl]-4-methyl-5-pyrimidinecarboxylate

A solution of ethyl 2-(diethoxymethyl)-4-methyl-5-pyrimidinecarboxylate (i.e. the product from Step A) (2 g, 0.00746 mol), hydroxylamine hydrochloride (1 g, 0.014 mol), ethanol (10 mL) and water (1 mL) was heated at reflux for 2 h, and then the reaction mixture was concentrated under reduced pressure. The residue was dissolved in water (10 mL), neutralized with aqueous NaHCO₃ solution and extracted with dichloromethane (2×20 mL). The dichloromethane solution was dried (MgSO₄) and concentrated under reduced pressure to provide the title compound as a brown solid (1.2 g).

¹H NMR (CDCl₃): δ 9.2 (br s, 1H), 8.4 (s, 1H), 4.43 (q, 2H), 2.86 (s, 3H), 1.42 (t, 3H).

### Step C: Preparation of 1,3-dichloro-5-[1-(trifluoromethyl)ethenyl]benzene

To a mixture of tetrahydrofuran (33 mL), ethylene glycol dimethyl ether (33 mL), and 4 N aqueous potassium hydroxide (33 mL) in a 200 mL Fisher-Porter sealed tube was added 3,5-dichlorophenyl boronic acid (8.72 g, 45.7 mmol) and 2-bromo-3,3,3-trifluoropropene (10.0 g, 57.2 mmol), followed by the addition of tetrakis(triphenylphosphine)palladium (0) (264 mg, 0.229 mmol). The mixture was heated to 75° C. for 3 h. The reaction mixture was then partitioned between diethyl ether and water. The aqueous layer was extracted with diethyl ether (2×20 mL). The organic extracts were combined, dried (MgSO₄), and concentrated under reduced pressure to provide a residue. The residue was purified by silica gel chromatography to afford the title compound as a clear oil (4.421 g).

¹H NMR (CDCl₃): δ 7.41 (s, 2H), 7.33 (s, 1H), 6.04 (d, 1H), 5.82 (d, 1H).

### Step D: Preparation of ethyl 2-[5-(3,5-dichlorophenyl)-4,5-dihydro-5-(trifluoromethyl)-3-isoxazolyl]-4-methyl-5-pyrimidinecarboxylate

A solution of Clorox® brand aqueous sodium hypochlorite (6.15%, 1.4 mL) and 1 N aqueous NaOH (0.1 mL) was added dropwise to a mixture of ethyl 2-[(hydroxyimino)methyl]-4-methyl-5-pyrimidinecarboxylate (i.e. the product from Step B) (0.21 g, 1 mmol) and 1,3-dichloro-5-[1-(trifluoromethyl)ethenyl]benzene (i.e. the product from Step C) (0.241 g, 1 mmol) in tetrahydrofuran (5 mL) and diethyl ether (5 mL) at 5° C. The reaction mixture was then stirred at room temperature for 1 h. The resulting mixture was poured into water (20 mL) and extracted with ethyl acetate (2×20 mL). The organic extracts were combined, dried (MgSO₄) and concentrated under reduced pressure to provide an oil, which was purified by chromatography on a silica gel column eluted with 10% ethyl acetate in hexanes to provide the title compound as a light yellow oil (30 mg).

¹H NMR (CDCl₃): δ 9.18 (s, 1H), 7.52 (m, 2H), 7.4 (s, 1H), 4.44 (q, 2H), 4.2 (d, 1H), 3.8 (d, 1H), 2.87 (s, 3H), 1.43 (t, 3H).

### Step E: Preparation of 2-[5-(3,5-dichlorophenyl)-4,5-dihydro-5-(trifluoromethyl)-3-isoxazolyl]-4-methyl-5-pyrimidinecarbonyl Chloride

A mixture of ethyl 2-[5-(3,5-dichlorophenyl)-4,5-dihydro-5-(trifluoromethyl)-3-isoxazolyl]-4-methyl-5-pyrimidinecarboxylate (i.e. the product from Step D) (0.15 g, 0.33 mmol) and 1 N aqueous sodium hydroxide (2 mL) in tetrahydrofuran (1 mL) was stirred at room temperature for 18 h. The resulting mixture was acidified with 1 N aqueous hydrochloric acid (3 mL) and extracted with dichloromethane (2×20 mL). The organic extracts were combined, dried (MgSO₄) and concentrated under reduced pressure to provide 2-[5-(3,5-dichlorophenyl)-4,5-dihydro-5-(trifluoromethyl)-3-isoxazolyl]-4-methyl-5-pyrimidinecarboxylic acid (0.1 g). This crude acid was dissolved in dichloromethane (2 mL), and oxalyl chloride (0.038 g, 0.3 mmol) was added followed by a drop of N,N-dimethylformamide. The mixture was stirred at room temperature for 0.5 h and then concentrated under reduced pressure to provide the title compound as an oil, which was used directly in the next step.

### Step F: Preparation of 2-[5-(3,5-dichlorophenyl)-4,5-dihydro-5-(trifluoromethyl)-3-isoxazolyl]-4-methyl-N-(2,2,2-trifluoroethyl)-5-pyrimidinecarboxamide

A solution of 2-[5-(3,5-dichlorophenyl)-4,5-dihydro-5-(trifluoromethyl)-3-isoxazolyl]-4-methyl-5-pyrimidinecarbonyl chloride (i.e. the product from Step E) in dichloromethane (2 mL) was added to a solution of 2,2,2-trifluoroethylamine (0.038 g, 0.388 mmol) and triethylamine (0.032 g, 0.032 mmol) in dichloromethane (3 mL) at 5° C. The mixture was then stirred at room temperature for 18 h. The mixture was concentrated under reduced pressure to provide a residue, which was purified by chromatography on silica gel eluted with 20% ethyl acetate in hexanes to provide the title compound, a compound of the present invention, as an oil (70 mg).

¹H NMR (CDCl₃): δ 8.76 (s, 1H), 7.51 (m, 2H), 7.4 (m, 1H), 6.3 (br s, 1H), 4.23 (q, 2H), 4.2 (m, 2H), 3.8 (d, 1H), 2.72 (s, 3H).

### EXAMPLE 2

### Preparation of 2-[5-(3,5-dichlorophenyl)-4,5-dihydro-5-(trifluoromethyl)-3-isoxazolyl]-4-methyl-N-(2-pyridinylmethyl)-5-pyrimidinecarboxamide

### Step A: Preparation of 2-(diethoxymethyl)-4-methyl-5-pyrimidinecarboxylic Acid

To a solution of ethyl 2-(diethoxymethyl)-4-methyl-5-pyrimidinecarboxylate (i.e. the product from Step A, Example 1) (2.68 g, 0.01 mol) in tetrahydrofuran (15 mL) was added 1 N aqueous sodium hydroxide (15 mL). The reaction mixture was stirred at room temperature for 18 h, then extracted with diethyl ether (2×10 mL), and the aqueous layer was acidified with 1 N aqueous hydrochloric acid to pH 5 and extracted with dichloromethane (2×10 mL). The dichloromethane extracts were dried (MgSO₄) and concentrated under reduced pressure to provide the title compound as a white solid (2 g).

¹H NMR (CDCl₃): δ 9.33 (s, 1H), 5.6 (s, 1H), 3.8 (q, 2H), 3.77 (q, 2H), 2.92 (s, 3H), 1.3 (t, 3H), 1.28 (t, 3H).

### Step B: Preparation of 2-(diethoxymethyl)-4-methyl-5-pyrimidinecarbonyl Chloride

To a solution of 2-(diethoxymethyl)-4-methyl-5-pyrimidinecarboxylic acid (i.e. the product from Step A) (1.2 g, 5.02 mmol) in dichloromethane (2 mL) was added oxalyl chloride (0.65 g, 5.2 mmol), followed by a drop of N,N-dimethylformamide. The mixture was stirred at room temperature for 0.5 h. The mixture was concentrated under reduced pressure to provide the title compound as an oil, which was used directly in the next step.

### Step C: Preparation of 2-(diethoxymethyl)-4-methyl-N-(2-pyridinylmethyl)-5-pyrimidinecarboxamide

To a solution of 2-(diethoxymethyl)-4-methyl-5-pyrimidinecarbonyl chloride (i.e. the product from Step B) in dichloromethane (2 mL) was added to a solution of 2-pyridinemethanamine (also known as 2-(aminomethyl)-pyridine) (0.6 g, 5.55 mmol) and triethylamine (0.52 g, 5.14 mmol) in dichloromethane (3 mL) at 5° C. The reaction mixture was then stirred at room temperature for 18 h. The resulting mixture was concentrated under reduced pressure to provide a residue, which was purified by chromatography on a silica gel column eluted with ethyl acetate to provide the title compound as a light yellow solid (1.5 g).

¹H NMR (CDCl₃): δ 8.84 (s, 1H), 8.6 (d, 1H), 7.8 (m, 1H), 7.6 (br s, 1H), 7.3 (s, 1H), 7.2 (m, 1H), 5.55 (m, 1H), 4.6 (d, 2H), 3.8 (q, 2H), 3.7 (q, 2H), 2.74 (s, 3H), 1.29 (m, 6H).

### Step D: Preparation of 2-[(hydroxyimino)methyl]-4-methyl-N-(2-pyridinylmethyl)-5-pyrimidinecarboxamide

A mixture of 2-(diethoxymethyl)-4-methyl-N-(2-pyridinylmethyl)-5-pyrimidinecarboxamide (i.e. the product from Step C) (0.30 g, 1 mmol) and hydroxylamine hydrochloride (0.15 g, 2.0 mmol) in ethanol (10 mL) was heated at reflux for 1 h. Additional hydroxylamine hydrochloride (0.15 g, 2.00 mmol) was added, and heating was continued for 10 minutes, then sodium acetate (30 mg, catalytic amount) was added, and the reaction mixture was heated for another 30 minutes. The mixture was allowed to come to room temperature, and diluted with water (10 mL), neutralized with solid sodium bicarbonate, and extracted with dichloromethane (2×20 mL). The organic extracts were dried (MgSO₄) and concentrated under reduced pressure to provide the title compound as a white solid (0.21 g).

¹H NMR (DMSO-d₆): δ 10.2 (s, 1H), 9.2 (m, 1H), 8.8 (s, 1H), 8.5 (m, 1H), 8.00 (s, 1H), 7.8 (s, 1H), 7.4 (m, 1H), 7.2 (m, 1H), 4.6 (d, 2H), 2.57 (s, 3H).

### Step E: Preparation of 2-[5-(3,5-dichlorophenyl)-4,5-dihydro-5-(trifluoromethyl)-3-isoxazolyl]-4-methyl-N-(2-pyridinylmethyl)-5-pyrimidinecarboxamide

To a solution of 2-[(hydroxyimino)methyl]-4-methyl-N-(2-pyridinylmethyl)-5-pyrimidinecarboxamide (i.e. the product from Step D) (0.135 g, 0.50 mmol) and 1,3-dichloro-5-[1-(trifluoromethyl)ethenyl]benzene (i.e. the product from Step C, Example 1) (0.135 g, 0.55 mmol) in dichloromethane (5 mL) at 5° C. was added Clorox® brand aqueous sodium hypochlorite (6.15%, 2.75 mL) and then triethylamine (3 drops). The reaction mixture was stirred for 1 h at room temperature. The mixture was then diluted with water (10 mL) and extracted with dichloromethane (2×20 mL). The organic extracts were dried (MgSO₄) and concentrated under reduced pressure to provide a solid residue, which was purified by silica gel column chromatography (ethyl acetate) to provide the title product, a compound of the present invention, as a solid (21 mg).

¹H NMR (CDCl₃): δ 8.9 (s, 1H), 8.6 (d, 1H), 7.8 (m, 1H), 7.5 (m, 2H), 7.4 (s, 1H), 7.2 (m, 1H), 7.1 (m, 1H), 4.8 (d, 2H), 4.25 (d, 1H), 3.88 (d, 1H), 2.77 (s, 3H).

### EXAMPLE 3

### Preparation of 2-[5-(3,5-dichlorophenyl)-4,5-dihydro-5-(trifluoromethyl)-3-isoxazolyl]-5-(1H-1,2,4-triazol-1-yl)-4-(trifluoromethyl)pyrimidine

### Step A: Preparation of 4-(dimethylamino)-1,1,1-trifluoro-3-(1H-1,2,4-triazol-1-yl)-3-buten-2-one

A mixture of 3-bromo-1,1,1′-trifluoro-2-propanone (4.00 g, 0.02 mol) and 1H-1,2,4-triazole (1.7 g, 0.02 mol) in isopropanol (20 mL) was heated at reflux for 4 h, and then concentrated under reduced pressure to provide a solid. To a solution of the solid dissolved in water (5 mL) and concentrated hydrochloric acid (3.63 mL) was added a solution of sodium nitrite (1.6 g, 0.024 mol) in water (5 mL) dropwise over 15 minutes at 5° C. The reaction mixture was stirred for 1 h at room temperature, then water (25 mL) was added and stirring was continued for an additional 15 minutes. The aqueous mixture was extracted with ethyl acetate (2×25 mL), and the combined organic extracts were dried (MgSO₄), filtered and concentrated under reduced pressure to give a solid. A mixture of the solid (3.00 g, 0.016 mol) and N,N-dimethylformamide dimethylacetal (2.5 mL, 0.018 mol) was heated at reflux for 2 h and then concentrated under reduced pressure to provide the title compound as a yellow oil (3.2 g).

¹H NMR (CDCl₃): δ 8.16 (s, 1H), 8.09 (s, 1H), 8.02 (s, 1H), 2.95 (s, 3H), 2.80 (3H).

### Step B: Preparation of 2-(diethoxymethyl)-5-(1H-1,2,4-triazol-1-yl)-4-(trifluoromethyl)pyrimidine

To a solution of 4-(dimethylamino)-1,1,1-trifluoro-3-(1H-1,2,4-triazol-1-yl)-3-buten-2-one (i.e. the product from Step A) (2.34 g, 0.01 mol) in ethanol (10 mL) was added a mixture of 2,2-diethoxyethanimidamide monohydrochloride (see J. Org. Chem. 1961, 26, 412 for preparation) (1.85 g, 0.01 mol) and sodium ethoxide (2.2 mL of 21% in ethanol). The mixture was heated to reflux for 5 h and then concentrated under reduced pressure. The resulting residue was suspended in water and extracted with dichloromethane (2×25 mL). The combined organic extracts were dried (MgSO₄), filtered and concentrated under reduced pressure to give a residue, which was purified by silica gel column chromatography eluted with dichloromethane-ethyl acetate (8:2) to provide the title compound as a yellow oil (0.591 g).

¹H NMR (CDCl₃): δ 9.20 (s, 1H), 8.40 (s, 1H), 8.20 (s, 1H), 5.54 (s, 1H), 3.80 (m, 2H), 3.70 (m, 2H), 1.26 (m, 6H).

### Step C: Preparation of 5-(1H-1,2,4-triazol-1-yl)-4-(trifluoromethyl)-2-pyrimidinecarboxaldehyde Oxime

A mixture of ethyl 2-(diethoxymethyl)-5-(1H-1,2,4-triazol-1-yl)-4-(trifluoromethyl)pyrimidine (i.e. the product from Step B) (0.59 g 1.86 mmol), hydroxylamine hydrochloride (0.624 g, 9.32 mmol), ethanol (10 mL) and 3 N hydrochloric acid (2 drops) was heated at reflux for 7 h, and then the reaction mixture was concentrated under reduced pressure. The resulting residue was dissolved in water (20 mL) and extracted with dichloromethane (2×25 mL). The combined organic extracts were dried (MgSO₄), filtered and concentrated under reduced pressure to provide the title compound as a solid (0.25 g).

¹H NMR (CDCl₃): δ 10.2 (s, 1H), 9.2 (m, 1H), 8.4 (s, 1H) 8.26 (s, 1H).

### Step D: Preparation of ethyl 2-[5-(3,5-dichlorophenyl)-4,5-dihydro-5-(trifluoromethyl)-3-isoxazolyl]-5-(1H-1,2,4-triazol-1-yl)-4-(trifluoromethyl)pyrimidine

A mixture of 5-(1H-1,2,4-triazol-1-yl)-4-(trifluoromethyl)-2-pyrimidinecarboxaldehyde oxime (i.e. the product of Step C) (0.25 g, 0.097 mmol), 1,3-dichloro-5-[1-(trifluoromethylethenyl]benzene (i.e. the product of Step C, Example 1) (0.163 g, 0.66 mmol), N-chlorosuccinimide (0.09 g, 0.67 mmol), potassium carbonate (0.303 g, 3.03 mmol) and ethyl acetate (10 mL) was heated at reflux. After 15 h, the reaction mixture was allowed to cool to room temperature and filtered, and the filtrate was concentrated under reduced pressure to provide a solid. The solid was purified by silica gel column chromatography eluted with ethyl acetate-hexanes (3:7) to provide the title compound, a compound of the present invention, as a white solid (0.11 mg).

¹H NMR (CDCl₃): δ 9.24 (s, 1H), 8.50 (s, 1H), 8.25 (s, 1H), 7.54 (m, 2H), 7.53 (m, 1H), 4.30 (d, 1H), 3.95 (d, 1H).

By the procedures described herein together with methods known in the art, the following compounds of Tables 1 to 3 can be prepared. The following abbreviations are used in the Tables which follow: t means tertiary, s means secondary, i means iso, n means normal, Me means methyl, Et means ethyl, Pr means propyl, Bu means butyl, i-Pr means isopropyl, s-Bu means sec butyl, i-Bu means isobutyl, t-Bu means tert-butyl, Ph means phenyl, OMe means methoxy, SMe means methylthio, CF₃ means trifluoromethyl, 2,4-di-F-Ph means 2,4-di-fluoro-phenyl, —CN means cyano and —NO₂ means nitro.

TABLE 1

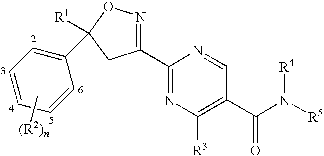

| R¹ | (R²)ₙ | R³ | R⁴ | R⁵ |
|---|---|---|---|---|
| CF₃ | H | Et | H | CH₂-2-pyridinyl |
| CF₃ | 2-Cl | Et | H | CH₂-2-pyridinyl |
| CF₃ | 3-Cl | Et | H | CH₂-2-pyridinyl |
| CF₃ | 4-Cl | Et | H | CH₂-2-pyridinyl |
| CF₃ | 2-Cl, 4-Cl | Et | H | CH₂-2-pyridinyl |
| CF₃ | 3-Cl, 4-Cl | Et | H | CH₂-2-pyridinyl |
| CF₃ | 3-Cl, 5-Cl | Et | H | CH₂-2-pyridinyl |
| CF₃ | 2-F | Et | H | CH₂-2-pyridinyl |
| CF₃ | 3-F | Et | H | CH₂-2-pyridinyl |
| CF₃ | 4-F | Et | H | CH₂-2-pyridinyl |
| CF₃ | 2-F, 4-F | Et | H | CH₂-2-pyridinyl |
| CF₃ | 3-F, 4-F | Et | H | CH₂-2-pyridinyl |
| CF₃ | 3-F, 5-F | Et | H | CH₂-2-pyridinyl |
| CF₃ | 3-CF₃ | Et | H | CH₂-2-pyridinyl |
| CF₃ | 4-CF₃ | Et | H | CH₂-2-pyridinyl |
| CF₃ | 3-F₃, 5-CF₃ | Et | H | CH₂-2-pyridinyl |
| CF₃ | 3-Br | Et | H | CH₂-2-pyridinyl |
| CF₃ | 4-Br | Et | H | CH₂-2-pyridinyl |
| CF₃ | 3-I | Et | H | CH₂-2-pyridinyl |
| CF₃ | 4-I | Et | H | CH₂-2-pyridinyl |
| CF₃ | 3-CN | Et | H | CH₂-2-pyridinyl |
| CF₃ | 4-CN | Et | H | CH₂-2-pyridinyl |
| CF₃ | 3-Me | Et | H | CH₂-2-pyridinyl |
| CF₃ | 4-Me | Et | H | CH₂-2-pyridinyl |
| CF₃ | 3-OMe | Et | H | CH₂-2-pyridinyl |
| CF₃ | 4-OMe | Et | H | CH₂-2-pyridinyl |
| CF₃ | 3-OCF₃ | Et | H | CH₂-2-pyridinyl |
| CF₃ | 4-OCF₃ | Et | H | CH₂-2-pyridinyl |
| CF₃ | H | Me | H | CH₂-2-pyridinyl |
| CF₃ | 2-Cl | Me | H | CH₂-2-pyridinyl |
| CF₃ | 3-Cl | Me | H | CH₂-2-pyridinyl |
| CF₃ | 4-Cl | Me | H | CH₂-2-pyridinyl |
| CF₃ | 2-Cl, 4-Cl | Me | H | CH₂-2-pyridinyl |
| CF₃ | 3-Cl, 4-Cl | Me | H | CH₂-2-pyridinyl |
| CF₃ | 3-Cl, 5-Cl | Me | H | CH₂-2-pyridinyl |
| CF₃ | 2-F | Me | H | CH₂-2-pyridinyl |
| CF₃ | 3-F | Me | H | CH₂-2-pyridinyl |
| CF₃ | 4-F | Me | H | CH₂-2-pyridinyl |
| CF₃ | 2-F, 4-F | Me | H | CH₂-2-pyridinyl |
| CF₃ | 3-F, 4-F | Me | H | CH₂-2-pyridinyl |
| CF₃ | 3-F, 5-F | Me | H | CH₂-2-pyridinyl |
| CF₃ | 3-CF₃ | Me | H | CH₂-2-pyridinyl |
| CF₃ | 4-CF₃ | Me | H | CH₂-2-pyridinyl |
| CF₃ | 3-CF₃, 5-CF₃ | Me | H | CH₂-2-pyridinyl |
| CF₃ | 3-Br | Me | H | CH₂-2-pyridinyl |
| CF₃ | 4-Br | Me | H | CH₂-2-pyridinyl |
| CF₃ | 3-I | Me | H | CH₂-2-pyridinyl |
| CF₃ | 4-I | Me | H | CH₂-2-pyridinyl |
| CF₃ | 3-CN | Me | H | CH₂-2-pyridinyl |
| CF₃ | 4-CN | Me | H | CH₂-2-pyridinyl |
| CF₃ | 3-Me | Me | H | CH₂-2-pyridinyl |
| CF₃ | 4-Me | Me | H | CH₂-2-pyridinyl |
| CF₃ | 3-OMe | Me | H | CH₂-2-pyridinyl |
| CF₃ | 4-OMe | Me | H | CH₂-2-pyridinyl |
| CF₃ | 3-OCF₃ | Me | H | CH₂-2-pyridinyl |
| CF₃ | 4-OCF₃ | Me | H | CH₂-2-pyridinyl |
| CF₃ | H | Me | CO₂Me | CH₂-2-pyridinyl |
| CF₃ | 2-Cl | Me | CO₂Me | CH₂-2-pyridinyl |
| CF₃ | 3-Cl | Me | CO₂Me | CH₂-2-pyridinyl |
| CF₃ | 4-Cl | Me | CO₂Me | CH₂-2-pyridinyl |
| CF₃ | 2-Cl, 4-Cl | Me | CO₂Me | CH₂-2-pyridinyl |
| CF₃ | 3-Cl, 4-Cl | Me | CO₂Me | CH₂-2-pyridinyl |
| CF₃ | 3-Cl, 5-Cl | Me | CO₂Me | CH₂-2-pyridinyl |
| CF₃ | 2-F | Me | CO₂Me | CH₂-2-pyridinyl |
| CF₃ | 3-F | Me | CO₂Me | CH₂-2-pyridinyl |
| CF₃ | 4-F | Me | CO₂Me | CH₂-2-pyridinyl |
| CF₃ | 2-F, 4-F | Me | CO₂Me | CH₂-2-pyridinyl |
| CF₃ | 3-F, 4-F | Me | CO₂Me | CH₂-2-pyridinyl |
| CF₃ | 3-F, 5-F | Me | CO₂Me | CH₂-2-pyridinyl |
| CF₃ | 3-CF₃ | Me | CO₂Me | CH₂-2-pyridinyl |
| CF₃ | 4-CF₃ | Me | CO₂Me | CH₂-2-pyridinyl |
| CF₃ | 3-CF₃, 5-CF₃ | Me | CO₂Me | CH₂-2-pyridinyl |
| CF₃ | 3-Br | Me | CO₂Me | CH₂-2-pyridinyl |
| CF₃ | 4-Br | Me | CO₂Me | CH₂-2-pyridinyl |
| CF₃ | 3-I | Me | CO₂Me | CH₂-2-pyridinyl |
| CF₃ | 4-I | Me | CO₂Me | CH₂-2-pyridinyl |
| CF₃ | 3-CN | Me | CO₂Me | CH₂-2-pyridinyl |
| CF₃ | 4-CN | Me | CO₂Me | CH₂-2-pyridinyl |
| CF₃ | 3-Me | Me | CO₂Me | CH₂-2-pyridinyl |
| CF₃ | 4-Me | Me | CO₂Me | CH₂-2-pyridinyl |
| CF₃ | 3-OMe | Me | CO₂Me | CH₂-2-pyridinyl |
| CF₃ | 4-OMe | Me | CO₂Me | CH₂-2-pyridinyl |
| CF₃ | 3-OCF₃ | Me | CO₂Me | CH₂-2-pyridinyl |
| CF₃ | 4-OCF₃ | Me | CO₂Me | CH₂-2-pyridinyl |
| CF₃ | H | Et | H | CH₂CF₃ |
| CF₃ | 2-Cl | Et | H | CH₂CF₃ |
| CF₃ | 3-Cl | Et | H | CH₂CF₃ |
| CF₃ | 4-Cl | Et | H | CH₂CF₃ |
| CF₃ | 2-Cl, 4-Cl | Et | H | CH₂CF₃ |
| CF₃ | 3-Cl, 4-Cl | Et | H | CH₂CF₃ |
| CF₃ | 3-Cl, 5-Cl | Et | H | CH₂CF₃ |
| CF₃ | 2-F | Et | H | CH₂CF₃ |
| CF₃ | 3-F | Et | H | CH₂CF₃ |
| CF₃ | 4-F | Et | H | CH₂CF₃ |
| CF₃ | 2-F, 4-F | Et | H | CH₂CF₃ |
| CF₃ | 3-F, 4-F | Et | H | CH₂CF₃ |
| CF₃ | 3-F, 5-F | Et | H | CH₂CF₃ |
| CF₃ | 3-CF₃ | Et | H | CH₂CF₃ |
| CF₃ | 4-CF₃ | Et | H | CH₂CF₃ |
| CF₃ | 3-CF₃, 5-CF₃ | Et | H | CH₂CF₃ |
| CF₃ | 3-Br | Et | H | CH₂CF₃ |
| CF₃ | 4-Br | Et | H | CH₂CF₃ |
| CF₃ | 3-I | Et | H | CH₂CF₃ |
| CF₃ | 4-I | Et | H | CH₂CF₃ |
| CF₃ | 3-CN | Et | H | CH₂CF₃ |
| CF₃ | 4-CN | Et | H | CH₂CF₃ |
| CF₃ | 3-Me | Et | H | CH₂CF₃ |
| CF₃ | 4-Me | Et | H | CH₂CF₃ |
| CF₃ | 3-OMe | Et | H | CH₂CF₃ |
| CF₃ | 4-OMe | Et | H | CH₂CF₃ |
| CF₃ | 3-OCF₃ | Et | H | CH₂CF₃ |
| CF₃ | 4-OCF₃ | Et | H | CH₂CF₃ |
| CF₃ | H | Me | CO₂Me | CH₂CF₃ |
| CF₃ | 2-Cl | Me | CO₂Me | CH₂CF₃ |
| CF₃ | 3-Cl | Me | CO₂Me | CH₂CF₃ |
| CF₃ | 4-Cl | Me | CO₂Me | CH₂CF₃ |
| CF₃ | 2-Cl, 4-Cl | Me | CO₂Me | CH₂CF₃ |
| CF₃ | 3-Cl, 4-Cl | Me | CO₂Me | CH₂CF₃ |
| CF₃ | 3-Cl, 5-Cl | Me | CO₂Me | CH₂CF₃ |
| CF₃ | 2-F | Me | CO₂Me | CH₂CF₃ |
| CF₃ | 3-F | Me | CO₂Me | CH₂CF₃ |
| CF₃ | 4-F | Me | CO₂Me | CH₂CF₃ |
| CF₃ | 2-F, 4-F | Me | CO₂Me | CH₂CF₃ |
| CF₃ | 3-F, 4-F | Me | CO₂Me | CH₂CF₃ |
| CF₃ | 3-F, 5-F | Me | CO₂Me | CH₂CF₃ |
| CF₃ | 3-CF₃ | Me | CO₂Me | CH₂CF₃ |
| CF₃ | 4-CF₃ | Me | CO₂Me | CH₂CF₃ |
| CF₃ | 3-CF₃, 5-CF₃ | Me | CO₂Me | CH₂CF₃ |
| CF₃ | 3-Br | Me | CO₂Me | CH₂CF₃ |
| CF₃ | 4-Br | Me | CO₂Me | CH₂CF₃ |
| CF₃ | 3-I | Me | CO₂Me | CH₂CF₃ |
| CF₃ | 4-I | Me | CO₂Me | CH₂CF₃ |
| CF₃ | 3-CN | Me | CO₂Me | CH₂CF₃ |
| CF₃ | 4-CN | Me | CO₂Me | CH₂CF₃ |
| CF₃ | 3-Me | Me | CO₂Me | CH₂CF₃ |
| CF₃ | 4-Me | Me | CO₂Me | CH₂CF₃ |
| CF₃ | 3-OMe | Me | CO₂Me | CH₂CF₃ |
| CF₃ | 4-OMe | Me | CO₂Me | CH₂CF₃ |
| CF₃ | 3-OCF₃ | Me | CO₂Me | CH₂CF₃ |
| CF₃ | 4-OCF₃ | Me | CO₂Me | CH₂CF₃ |
| Me | H | Me | H | CH₂CF₃ |
| Me | 2-Cl | Me | H | CH₂CF₃ |
| Me | 3-Cl | Me | H | CH₂CF₃ |
| Me | 4-Cl | Me | H | CH₂CF₃ |
| Me | 2-Cl, 4-Cl | Me | H | CH₂CF₃ |
| Me | 3-Cl, 4-Cl | Me | H | CH₂CF₃ |
| Me | 3-Cl, 5-Cl | Me | H | CH₂CF₃ |
| Me | 2-F | Me | H | CH₂CF₃ |
| Me | 3-F | Me | H | CH₂CF₃ |
| Me | 4-F | Me | H | CH₂CF₃ |
| Me | 2-F, 4-F | Me | H | CH₂CF₃ |
| Me | 3-F, 4-F | Me | H | CH₂CF₃ |
| Me | 3-F, 5-F | Me | H | CH₂CF₃ |
| Me | 3-CF₃ | Me | H | CH₂CF₃ |
| Me | 4-CF₃ | Me | H | CH₂CF₃ |
| Me | 3-CF₃, 5-CF₃ | Me | H | CH₂CF₃ |
| Me | 3-Br | Me | H | CH₂CF₃ |
| Me | 4-Br | Me | H | CH₂CF₃ |
| Me | 3-I | Me | H | CH₂CF₃ |
| Me | 4-I | Me | H | CH₂CF₃ |
| Me | 3-CN | Me | H | CH₂CF₃ |
| Me | 4-CN | Me | H | CH₂CF₃ |
| Me | 3-Me | Me | H | CH₂CF₃ |
| Me | 4-Me | Me | H | CH₂CF₃ |
| Me | 3-OMe | Me | H | CH₂CF₃ |
| Me | 4-OMe | Me | H | CH₂CF₃ |
| Me | 3-OCF₃ | Me | H | CH₂CF₃ |
| Me | 4-OCF₃ | Me | H | CH₂CF₃ |
| CF₃ | H | Ph | H | CH₂CF₃ |
| CF₃ | 2-Cl | Ph | H | CH₂CF₃ |
| CF₃ | 3-Cl | Ph | H | CH₂CF₃ |
| CF₃ | 4-Cl | Ph | H | CH₂CF₃ |
| CF₃ | 2-Cl, 4-Cl | Ph | H | CH₂CF₃ |
| CF₃ | 3-Cl, 4-Cl | Ph | H | CH₂CF₃ |
| CF₃ | 3-Cl, 5-Cl | Ph | H | CH₂CF₃ |
| CF₃ | 2-F | Ph | H | CH₂CF₃ |
| CF₃ | 3-F | Ph | H | CH₂CF₃ |
| CF₃ | 4-F | Ph | H | CH₂CF₃ |
| CF₃ | 2-F, 4-F | Ph | H | CH₂CF₃ |
| CF₃ | 3-F, 4-F | Ph | H | CH₂CF₃ |
| CF₃ | 3-F, 5-F | Ph | H | CH₂CF₃ |
| CF₃ | 3-CF₃ | Ph | H | CH₂CF₃ |
| CF₃ | 3-Cl, 5-Cl | Et | H | CH₂CH(Me)SMe |
| CF₃ | 3-Cl, 4-Cl | Et | H | CH₂CH(Me)SMe |
| CF₃ | 3-Cl, 5-Cl | i-Pr | H | CH₂CH(Me)SMe |
| CF₃ | 3-Cl, 4-Cl | i-Pr | H | CH₂CH(Me)SMe |
| CF₃ | 3-Cl, 5-Cl | Me | H | CH₂CH(Me)SMe |
| CF₃ | 3-Cl, 4-Cl | Me | H | CH₂CH(Me)SMe |
| CF₃ | 3-Cl, 5-Cl | Ph | H | CH₂CH(Me)SMe |
| CF₃ | 3-Cl, 4-Cl | Ph | H | CH₂CH(Me)SMe |
| CF₃ | 3-Cl, 5-Cl | Et | H | CH₂CN |
| CF₃ | 3-Cl, 4-Cl | Et | H | CH₂CN |
| CF₃ | 3-Cl, 5-Cl | i-Pr | H | CH₂CN |
| CF₃ | 3-Cl, 4-Cl | i-Pr | H | CH₂CN |
| CF₃ | 3-Cl, 5-Cl | Me | H | CH₂CN |
| CF₃ | 3-Cl, 4-Cl | Me | H | CH₂CN |
| CF₃ | 3-Cl, 5-Cl | Ph | H | CH₂CN |
| CF₃ | 3-Cl, 4-Cl | Ph | H | CH₂CN |
| CF₃ | 3-Cl, 5-Cl | Et | H | CH₂Ph |
| CF₃ | 3-Cl, 4-Cl | Et | H | CH₂Ph |
| CF₃ | 3-Cl, 5-Cl | i-Pr | H | CH₂Ph |
| CF₃ | 3-Cl, 4-Cl | i-Pr | H | CH₂Ph |
| CF₃ | 3-Cl, 5-Cl | Me | H | CH₂Ph |
| CF₃ | 3-Cl, 4-Cl | Me | H | CH₂Ph |
| CF₃ | 3-Cl, 5-Cl | Ph | H | CH₂Ph |
| CF₃ | 3-Cl, 4-Cl | Ph | H | CH₂Ph |
| CF₃ | 3-Cl, 5-Cl | Et | H | i-Bu |
| CF₃ | 3-Cl, 4-Cl | Et | H | i-Bu |
| CF₃ | 3-Cl, 5-Cl | i-Pr | H | i-Bu |
| CF₃ | 3-Cl, 4-Cl | i-Pr | H | i-Bu |
| CF₃ | 3-Cl, 5-Cl | Me | H | i-Bu |
| CF₃ | 3-Cl, 4-Cl | Me | H | i-Bu |
| CF₃ | 3-Cl, 5-Cl | Ph | H | i-Bu |
| CF₃ | 3-Cl, 4-Cl | Ph | H | i-Bu |
| CF₃ | 3-Cl, 5-Cl | Et | H | Me |
| CF₃ | 3-Cl, 4-Cl | Et | H | Me |
| CF₃ | 3-Cl, 5-Cl | i-Pr | H | Me |
| CF₃ | 3-Cl, 4-Cl | i-Pr | H | Me |
| CF₃ | 3-Cl, 5-Cl | Me | H | Me |
| CF₃ | 3-Cl, 4-Cl | Me | H | Me |
| CF₃ | 3-Cl, 5-Cl | Ph | H | Me |
| CF₃ | 3-Cl, 4-Cl | Ph | H | Me |
| CF₃ | 3-Cl, 5-Cl | Et | H | s-Bu |
| CF₃ | 3-Cl, 4-Cl | Et | H | s-Bu |
| CF₃ | 3-Cl, 5-Cl | i-Pr | H | s-Bu |
| CF₃ | 3-Cl, 4-Cl | i-Pr | H | s-Bu |
| CF₃ | H | i-Pr | H | CH₂-2-pyridinyl |
| CF₃ | 2-Cl | i-Pr | H | CH₂-2-pyridinyl |
| CF₃ | 3-Cl | i-Pr | H | CH₂-2-pyridinyl |
| CF₃ | 4-Cl | i-Pr | H | CH₂-2-pyridinyl |
| CF₃ | 2-Cl, 4-Cl | i-Pr | H | CH₂-2-pyridinyl |
| CF₃ | 3-Cl, 4-Cl | i-Pr | H | CH₂-2-pyridinyl |
| CF₃ | 3-Cl, 5-Cl | i-Pr | H | CH₂-2-pyridinyl |
| CF₃ | 2-F | i-Pr | H | CH₂-2-pyridinyl |
| CF₃ | 3-F | i-Pr | H | CH₂-2-pyridinyl |
| CF₃ | 4-F | i-Pr | H | CH₂-2-pyridinyl |
| CF₃ | 2-F, 4-F | i-Pr | H | CH₂-2-pyridinyl |
| CF₃ | 3-F, 4-F | i-Pr | H | CH₂-2-pyridinyl |
| CF₃ | 3-F, 5-F | i-Pr | H | CH₂-2-pyridinyl |
| CF₃ | 3-CF₃ | i-Pr | H | CH₂-2-pyridinyl |
| CF₃ | 4-CF₃ | i-Pr | H | CH₂-2-pyridinyl |
| CF₃ | 3-CF₃, 5-CF₃ | i-Pr | H | CH₂-2-pyridinyl |
| CF₃ | 3-Br | i-Pr | H | CH₂-2-pyridinyl |
| CF₃ | 4-Br | i-Pr | H | CH₂-2-pyridinyl |
| CF₃ | 3-I | i-Pr | H | CH₂-2-pyridinyl |
| CF₃ | 4-I | i-Pr | H | CH₂-2-pyridinyl |
| CF₃ | 3-CN | i-Pr | H | CH₂-2-pyridinyl |
| CF₃ | 4-CN | i-Pr | H | CH₂-2-pyridinyl |
| CF₃ | 3-Me | i-Pr | H | CH₂-2-pyridinyl |
| CF₃ | 4-Me | i-Pr | H | CH₂-2-pyridinyl |
| CF₃ | 3-OMe | i-Pr | H | CH₂-2-pyridinyl |
| CF₃ | 4-OMe | i-Pr | H | CH₂-2-pyridinyl |
| CF₃ | 3-OCF₃ | i-Pr | H | CH₂-2-pyridinyl |
| CF₃ | 4-OCF₃ | i-Pr | H | CH₂-2-pyridinyl |
| CF₃ | H | Me | Me | CH₂-2-pyridinyl |
| CF₃ | 2-Cl | Me | Me | CH₂-2-pyridinyl |
| CF₃ | 3-Cl | Me | Me | CH₂-2-pyridinyl |
| CF₃ | 4-Cl | Me | Me | CH₂-2-pyridinyl |
| CF₃ | 2-Cl, 4-Cl | Me | Me | CH₂-2-pyridinyl |
| CF₃ | 3-Cl, 4-Cl | Me | Me | CH₂-2-pyridinyl |
| CF₃ | 3-Cl, 5-Cl | Me | Me | CH₂-2-pyridinyl |
| CF₃ | 2-F | Me | Me | CH₂-2-pyridinyl |
| CF₃ | 3-F | Me | Me | CH₂-2-pyridinyl |
| CF₃ | 4-F | Me | Me | CH₂-2-pyridinyl |
| CF₃ | 2-F, 4-F | Me | Me | CH₂-2-pyridinyl |
| CF₃ | 3-F, 4-F | Me | Me | CH₂-2-pyridinyl |
| CF₃ | 3-F, 5-F | Me | Me | CH₂-2-pyridinyl |
| CF₃ | 3-CF₃ | Me | Me | CH₂-2-pyridinyl |
| CF₃ | 4-CF₃ | Me | Me | CH₂-2-pyridinyl |
| CF₃ | 3-CF₃, 5-CF₃ | Me | Me | CH₂-2-pyridinyl |
| CF₃ | 3-Br | Me | Me | CH₂-2-pyridinyl |
| CF₃ | 4-Br | Me | Me | CH₂-2-pyridinyl |
| CF₃ | 3-I | Me | Me | CH₂-2-pyridinyl |
| CF₃ | 4-I | Me | Me | CH₂-2-pyridinyl |
| CF₃ | 3-CN | Me | Me | CH₂-2-pyridinyl |
| CF₃ | 4-CN | Me | Me | CH₂-2-pyridinyl |
| CF₃ | 3-Me | Me | Me | CH₂-2-pyridinyl |
| CF₃ | 4-Me | Me | Me | CH₂-2-pyridinyl |
| CF₃ | 3-OMe | Me | Me | CH₂-2-pyridinyl |
| CF₃ | 4-OMe | Me | Me | CH₂-2-pyridinyl |
| CF₃ | 3-OCF₃ | Me | Me | CH₂-2-pyridinyl |
| CF₃ | 4-OCF₃ | Me | Me | CH₂-2-pyridinyl |
| CF₃ | H | Ph | H | CH₂-2-pyridinyl |
| CF₃ | 2-Cl | Ph | H | CH₂-2-pyridinyl |
| CF₃ | 3-Cl | Ph | H | CH₂-2-pyridinyl |
| CF₃ | 4-Cl | Ph | H | CH₂-2-pyridinyl |
| CF₃ | 2-Cl, 4-Cl | Ph | H | CH₂-2-pyridinyl |
| CF₃ | 3-Cl, 4-Cl | Ph | H | CH₂-2-pyridinyl |
| CF₃ | 3-Cl, 5-Cl | Ph | H | CH₂-2-pyridinyl |
| CF₃ | 2-F | Ph | H | CH₂-2-pyridinyl |
| CF₃ | 3-F | Ph | H | CH₂-2-pyridinyl |
| CF₃ | 4-F | Ph | H | CH₂-2-pyridinyl |
| CF₃ | 2-F, 4-F | Ph | H | CH₂-2-pyridinyl |
| CF₃ | 3-F, 4-F | Ph | H | CH₂-2-pyridinyl |
| CF₃ | 3-F, 5-F | Ph | H | CH₂-2-pyridinyl |
| CF₃ | 3-CF₃ | Ph | H | CH₂-2-pyridinyl |
| CF₃ | 4-CF₃ | Ph | H | CH₂-2-pyridinyl |
| CF₃ | 3-CF₃, 5-CF₃ | Ph | H | CH₂-2-pyridinyl |
| CF₃ | 3-Br | Ph | H | CH₂-2-pyridinyl |
| CF₃ | 4-Br | Ph | H | CH₂-2-pyridinyl |
| CF₃ | 3-I | Ph | H | CH₂-2-pyridinyl |
| CF₃ | 4-I | Ph | H | CH₂-2-pyridinyl |
| CF₃ | 3-CN | Ph | H | CH₂-2-pyridinyl |
| CF₃ | 4-CN | Ph | H | CH₂-2-pyridinyl |
| CF₃ | 3-Me | Ph | H | CH₂-2-pyridinyl |
| CF₃ | 4-Me | Ph | H | CH₂-2-pyridinyl |
| CF₃ | 3-OMe | Ph | H | CH₂-2-pyridinyl |
| CF₃ | 4-OMe | Ph | H | CH₂-2-pyridinyl |
| CF₃ | 3-OCF₃ | Ph | H | CH₂-2-pyridinyl |
| CF₃ | 4-OCF₃ | Ph | H | CH₂-2-pyridinyl |
| CF₃ | H | i-Pr | H | CH₂CF₃ |
| CF₃ | 2-Cl | i-Pr | H | CH₂CF₃ |
| CF₃ | 3-Cl | i-Pr | H | CH₂CF₃ |
| CF₃ | 4-Cl | i-Pr | H | CH₂CF₃ |
| CF₃ | 2-Cl, 4-Cl | i-Pr | H | CH₂CF₃ |
| CF₃ | 3-Cl, 4-Cl | i-Pr | H | CH₂CF₃ |
| CF₃ | 3-Cl, 5-Cl | i-Pr | H | CH₂CF₃ |
| CF₃ | 2-F | i-Pr | H | CH₂CF₃ |
| CF₃ | 3-F | i-Pr | H | CH₂CF₃ |
| CF₃ | 4-F | i-Pr | H | CH₂CF₃ |
| CF₃ | 2-F, 4-F | i-Pr | H | CH₂CF₃ |
| CF₃ | 3-F, 4-F | i-Pr | H | CH₂CF₃ |
| CF₃ | 3-F, 5-F | i-Pr | H | CH₂CF₃ |
| CF₃ | 3-CF₃ | i-Pr | H | CH₂CF₃ |
| CF₃ | 4-CF₃ | i-Pr | H | CH₂CF₃ |
| CF₃ | 3-CF₃, 5-CF₃ | i-Pr | H | CH₂CF₃ |
| CF₃ | 3-Br | i-Pr | H | CH₂CF₃ |
| CF₃ | 4-Br | i-Pr | H | CH₂CF₃ |
| CF₃ | 3-I | i-Pr | H | CH₂CF₃ |
| CF₃ | 4-I | i-Pr | H | CH₂CF₃ |
| CF₃ | 3-CN | i-Pr | H | CH₂CF₃ |
| CF₃ | 4-CN | i-Pr | H | CH₂CF₃ |
| CF₃ | 3-Me | i-Pr | H | CH₂CF₃ |
| CF₃ | 4-Me | i-Pr | H | CH₂CF₃ |
| CF₃ | 3-OMe | i-Pr | H | CH₂CF₃ |
| CF₃ | 4-OMe | i-Pr | H | CH₂CF₃ |
| CF₃ | 3-OCF₃ | i-Pr | H | CH₂CF₃ |
| CF₃ | 4-OCF₃ | i-Pr | H | CH₂CF₃ |
| CF₃ | H | Me | H | CH₂CF₃ |
| CF₃ | 2-Cl | Me | H | CH₂CF₃ |
| CF₃ | 3-Cl | Me | H | CH₂CF₃ |
| CF₃ | 4-Cl | Me | H | CH₂CF₃ |
| CF₃ | 2-Cl, 4-Cl | Me | H | CH₂CF₃ |
| CF₃ | 3-Cl, 4-Cl | Me | H | CH₂CF₃ |
| CF₃ | 3-Cl, 5-Cl | Me | H | CH₂CF₃ |
| CF₃ | 2-F | Me | H | CH₂CF₃ |
| CF₃ | 3-F | Me | H | CH₂CF₃ |
| CF₃ | 4-F | Me | H | CH₂CF₃ |
| CF₃ | 2-F, 4-F | Me | H | CH₂CF₃ |
| CF₃ | 3-F, 4-F | Me | H | CH₂CF₃ |
| CF₃ | 3-F, 5-F | Me | H | CH₂CF₃ |
| CF₃ | 3-CF₃ | Me | H | CH₂CF₃ |
| CF₃ | 4-CF₃ | Me | H | CH₂CF₃ |
| CF₃ | 3-CF₃, 5-CF₃ | Me | H | CH₂CF₃ |
| CF₃ | 3-Br | Me | H | CH₂CF₃ |
| CF₃ | 4-Br | Me | H | CH₂CF₃ |
| CF₃ | 3-I | Me | H | CH₂CF₃ |
| CF₃ | 4-I | Me | H | CH₂CF₃ |
| CF₃ | 3-CN | Me | H | CH₂CF₃ |
| CF₃ | 4-CN | Me | H | CH₂CF₃ |
| CF₃ | 3-Me | Me | H | CH₂CF₃ |
| CF₃ | 4-Me | Me | H | CH₂CF₃ |
| CF₃ | 3-OMe | Me | H | CH₂CF₃ |
| CF₃ | 4-OMe | Me | H | CH₂CF₃ |
| CF₃ | 3-OCF₃ | Me | H | CH₂CF₃ |
| CF₃ | 4-OCF₃ | Me | H | CH₂CF₃ |
| CF₃ | H | Me | Me | CH₂CF₃ |
| CF₃ | 2-Cl | Me | Me | CH₂CF₃ |
| CF₃ | 3-Cl | Me | Me | CH₂CF₃ |
| CF₃ | 4-Cl | Me | Me | CH₂CF₃ |
| CF₃ | 2-Cl, 4-Cl | Me | Me | CH₂CF₃ |
| CF₃ | 3-Cl, 4-Cl | Me | Me | CH₂CF₃ |
| CF₃ | 3-Cl, 5-Cl | Me | Me | CH₂CF₃ |
| CF₃ | 2-F | Me | Me | CH₂CF₃ |
| CF₃ | 3-F | Me | Me | CH₂CF₃ |
| CF₃ | 4-F | Me | Me | CH₂CF₃ |
| CF₃ | 2-F, 4-F | Me | Me | CH₂CF₃ |
| CF₃ | 3-F, 4-F | Me | Me | CH₂CF₃ |
| CF₃ | 3-F, 5-F | Me | Me | CH₂CF₃ |
| CF₃ | 3-CF₃ | Me | Me | CH₂CF₃ |
| CF₃ | 4-CF₃ | Me | Me | CH₂CF₃ |
| CF₃ | 3-CF₃, 5-CF₃ | Me | Me | CH₂CF₃ |
| CF₃ | 3-Br | Me | Me | CH₂CF₃ |
| CF₃ | 4-Br | Me | Me | CH₂CF₃ |
| CF₃ | 3-I | Me | Me | CH₂CF₃ |
| CF₃ | 4-I | Me | Me | CH₂CF₃ |
| CF₃ | 3-CN | Me | Me | CH₂CF₃ |
| CF₃ | 4-CN | Me | Me | CH₂CF₃ |
| CF₃ | 3-Me | Me | Me | CH₂CF₃ |
| CF₃ | 4-Me | Me | Me | CH₂CF₃ |
| CF₃ | 3-OMe | Me | Me | CH₂CF₃ |
| CF₃ | 4-OMe | Me | Me | CH₂CF₃ |
| CF₃ | 3-OCF₃ | Me | Me | CH₂CF₃ |
| CF₃ | 4-OCF₃ | Me | Me | CH₂CF₃ |
| CF₃ | 4-CF₃ | Ph | H | CH₂CF₃ |
| CF₃ | 3-CF₃, 5-CF₃ | Ph | H | CH₂CF₃ |
| CF₃ | 3-Br | Ph | H | CH₂CF₃ |
| CF₃ | 4-Br | Ph | H | CH₂CF₃ |
| CF₃ | 3-I | Ph | H | CH₂CF₃ |
| CF₃ | 4-I | Ph | H | CH₂CF₃ |
| CF₃ | 3-CN | Ph | H | CH₂CF₃ |
| CF₃ | 4-CN | Ph | H | CH₂CF₃ |
| CF₃ | 3-Me | Ph | H | CH₂CF₃ |
| CF₃ | 4-Me | Ph | H | CH₂CF₃ |
| CF₃ | 3-OMe | Ph | H | CH₂CF₃ |
| CF₃ | 4-OMe | Ph | H | CH₂CF₃ |
| CF₃ | 3-OCF₃ | Ph | H | CH₂CF₃ |
| CF₃ | 4-OCF₃ | Ph | H | CH₂CF₃ |
| CF₃ | 3-Cl, 5-Cl | Et | H | CH₂CH₂SMe |
| CF₃ | 3-Cl, 4-Cl | Et | H | CH₂CH₂SMe |
| CF₃ | 3-Cl, 5-Cl | i-Pr | H | CH₂CH₂SMe |
| CF₃ | 3-Cl, 4-Cl | i-Pr | H | CH₂CH₂SMe |
| CF₃ | 3-Cl, 5-Cl | Me | H | CH₂CH₂SMe |
| CF₃ | 3-Cl, 4-Cl | Me | H | CH₂CH₂SMe |
| CF₃ | 3-Cl, 5-Cl | Ph | H | CH₂CH₂SMe |
| CF₃ | 3-Cl, 4-Cl | Ph | H | CH₂CH₂SMe |
| CF₃ | 3-Cl, 5-Cl | Et | H | CH₂CO₂Me |
| CF₃ | 3-Cl, 4-Cl | Et | H | CH₂CO₂Me |
| CF₃ | 3-Cl, 5-Cl | i-Pr | H | CH₂CO₂Me |
| CF₃ | 3-Cl, 4-Cl | i-Pr | H | CH₂CO₂Me |
| CF₃ | 3-Cl, 5-Cl | Me | H | CH₂CO₂Me |
| CF₃ | 3-Cl, 4-Cl | Me | H | CH₂CO₂Me |
| CF₃ | 3-Cl, 5-Cl | Ph | H | CH₂CO₂Me |
| CF₃ | 3-Cl, 4-Cl | Ph | H | CH₂CO₂Me |
| CF₃ | 3-Cl, 5-Cl | Et | H | Et |
| CF₃ | 3-Cl, 4-Cl | Et | H | Et |
| CF₃ | 3-Cl, 5-Cl | i-Pr | H | Et |
| CF₃ | 3-Cl, 4-Cl | i-Pr | H | Et |
| CF₃ | 3-Cl, 5-Cl | Me | H | Et |
| CF₃ | 3-Cl, 4-Cl | Me | H | Et |
| CF₃ | 3-Cl, 5-Cl | Ph | H | Et |
| CF₃ | 3-Cl, 4-Cl | Ph | H | Et |
| CF₃ | 3-Cl, 5-Cl | Et | H | i-Pr |
| CF₃ | 3-Cl, 4-Cl | Et | H | i-Pr |
| CF₃ | 3-Cl, 5-Cl | i-Pr | H | i-Pr |
| CF₃ | 3-Cl, 4-Cl | i-Pr | H | i-Pr |
| CF₃ | 3-Cl, 5-Cl | Me | H | i-Pr |
| CF₃ | 3-Cl, 4-Cl | Me | H | i-Pr |
| CF₃ | 3-Cl, 5-Cl | Ph | H | i-Pr |
| CF₃ | 3-Cl, 4-Cl | Ph | H | i-Pr |
| CF₃ | 3-Cl, 5-Cl | Et | H | n-Bu |
| CF₃ | 3-Cl, 4-Cl | Et | H | n-Bu |
| CF₃ | 3-Cl, 5-Cl | i-Pr | H | n-Bu |
| CF₃ | 3-Cl, 4-Cl | i-Pr | H | n-Bu |
| CF₃ | 3-Cl, 5-Cl | Me | H | n-Bu |
| CF₃ | 3-Cl, 4-Cl | Me | H | n-Ru |
| CF₃ | 3-Cl, 5-Cl | Ph | H | n-Bu |
| CF₃ | 3-Cl, 4-Cl | Ph | H | n-Bu |
| CF₃ | 3-Cl, 5-Cl | Me | H | s-Bu |
| CF₃ | 3-Cl, 4-Cl | Me | H | s-Bu |
| CF₃ | 3-Cl, 5-Cl | Ph | H | s-Bu |
| CF₃ | 3-Cl, 4-Cl | Ph | H | s-Bu |

TABLE 2

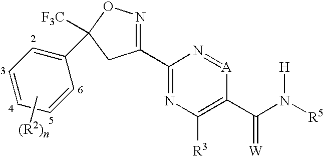

| W | (R²)ₙ | R³ | A | R⁵ |
|---|---|---|---|---|
| O | 3-Cl, 4-Cl | Br | CH | CH₂-2-pyridinyl |
| O | 3-Cl, 5-Cl | Br | CH | CH₂-2-pyridinyl |
| O | 3-Cl, 4-Cl | Cl | CH | CH₂-2-pyridinyl |
| O | 3-Cl, 5-Cl | Cl | CH | CH₂-2-pyridinyl |
| S | 3-Cl, 4-Cl | Et | CH | CH₂-2-pyridinyl |
| S | 3-Cl, 5-Cl | Et | CH | CH₂-2-pyridinyl |
| S | 3-Cl, 4-Cl | i-Pr | CH | CH₂-2-pyridinyl |
| S | 3-Cl, 5-Cl | i-Pr | CH | CH₂-2-pyridinyl |
| S | 3-Cl, 4-Cl | Me | CH | CH₂-2-pyridinyl |
| S | 3-Cl, 5-Cl | Me | CH | CH₂-2-pyridinyl |
| O | 3-Cl, 4-Cl | OMe | CH | CH₂-2-pyridinyl |
| O | 3-Cl, 5-Cl | OMe | CH | CH₂-2-pyridinyl |
| S | 3-Cl, 4-Cl | Ph | CH | CH₂-2-pyridinyl |
| S | 3-Cl, 5-Cl | Ph | CH | CH₂-2-pyridinyl |
| O | 3-Cl, 4-Cl | SMe | CH | CH₂-2-pyridinyl |
| O | 3-Cl, 5-Cl | SMe | CH | CH₂-2-pyridinyl |
| O | 3-Cl, 4-Cl | Et | N | CH₂-2-pyridinyl |
| O | 3-Cl, 5-Cl | Et | N | CH₂-2-pyridinyl |
| S | 3-Cl, 4-Cl | Et | N | CH₂-2-pyridinyl |
| S | 3-Cl, 5-Cl | Et | N | CH₂-2-pyridinyl |
| O | 3-Cl, 4-Cl | i-Pr | N | CH₂-2-pyridinyl |
| O | 3-Cl, 5-Cl | i-Pr | N | CH₂-2-pyridinyl |
| S | 3-Cl, 4-Cl | i-Pr | N | CH₂-2-pyridinyl |
| S | 3-Cl, 5-Cl | i-Pr | N | CH₂-2-pyridinyl |
| O | 3-Cl, 4-Cl | Me | N | CH₂-2-pyridinyl |
| O | 3-Cl, 5-Cl | Me | N | CH₂-2-pyridinyl |
| S | 3-Cl, 4-Cl | Me | N | CH₂-2-pyridinyl |
| S | 3-Cl, 5-Cl | Me | N | CH₂-2-pyridinyl |
| O | 3-Cl, 4-Cl | Ph | N | CH₂-2-pyridinyl |
| O | 3-Cl, 5-Cl | Ph | N | CH₂-2-pyridinyl |
| S | 3-Cl, 4-Cl | Ph | N | CH₂-2-pyridinyl |
| S | 3-Cl, 5-Cl | Ph | N | CH₂-2-pyridinyl |
| O | 3-Cl, 4-Cl | Et | C-Me | CH₂CF₃ |
| O | 3-Cl, 5-Cl | Et | C-Me | CH₂CF₃ |
| S | 3-Cl, 4-Cl | Et | C-Me | CH₂CF₃ |
| S | 3-Cl, 5-Cl | Et | C-Me | CH₂CF₃ |
| O | 3-Cl, 4-Cl | i-Pr | C-Me | CH₂CF₃ |
| O | 3-Cl, 5-Cl | i-Pr | C-Me | CH₂CF₃ |
| S | 3-Cl, 4-Cl | i-Pr | C-Me | CH₂CF₃ |
| S | 3-Cl, 5-Cl | i-Pr | C-Me | CH₂CF₃ |
| O | 3-Cl, 4-Cl | Me | C-Me | CH₂CF₃ |
| O | 3-Cl, 5-Cl | Me | C-Me | CH₂CF₃ |
| S | 3-Cl, 4-Cl | Me | C-Me | CH₂CF₃ |
| S | 3-Cl, 5-Cl | Me | C-Me | CH₂CF₃ |
| O | 3-Cl, 4-Cl | Ph | C-Me | CH₂CF₃ |
| O | 3-Cl, 5-Cl | Ph | C-Me | CH₂CF₃ |
| S | 3-Cl, 4-Cl | Ph | C-Me | CH₂CF₃ |
| S | 3-Cl, 5-Cl | Ph | C-Me | CH₂CF₃ |
| O | 3-Cl, 4-Cl | Et | C-Me | CH₂-2-pyridinyl |
| O | 3-Cl, 5-Cl | Et | C-Me | CH₂-2-pyridinyl |
| S | 3-Cl, 4-Cl | Et | C-Me | CH₂-2-pyridinyl |
| S | 3-Cl, 5-Cl | Et | C-Me | CH₂-2-pyridinyl |
| O | 3-Cl, 4-Cl | i-Pr | C-Me | CH₂-2-pyridinyl |
| O | 3-Cl, 5-Cl | i-Pr | C-Me | CH₂-2-pyridinyl |
| S | 3-Cl, 4-Cl | i-Pr | C-Me | CH₂-2-pyridinyl |
| S | 3-Cl, 5-Cl | i-Pr | C-Me | CH₂-2-pyridinyl |
| O | 3-Cl, 4-Cl | Me | C-Me | CH₂-2-pyridinyl |
| O | 3-Cl, 5-Cl | Me | C-Me | CH₂-2-pyridinyl |
| S | 3-Cl, 4-Cl | Me | C-Me | CH₂-2-pyridinyl |
| S | 3-Cl, 5-Cl | Me | C-Me | CH₂-2-pyridinyl |
| O | 3-Cl, 4-Cl | Ph | C-Me | CH₂-2-pyridinyl |
| O | 3-Cl, 5-Cl | Ph | C-Me | CH₂-2-pyridinyl |
| S | 3-Cl, 4-Cl | Ph | C-Me | CH₂-2-pyridinyl |
| S | 3-Cl, 5-Cl | Ph | C-Me | CH₂-2-pyridinyl |
| O | 3-Cl, 4-Cl | Br | CH | CH₂CF₃ |
| O | 3-Cl, 5-Cl | Br | CH | CH₂CF₃ |
| O | 3-Cl, 4-Cl | Cl | CH | CH₂CF₃ |
| O | 3-Cl, 5-Cl | Cl | CH | CH₂CF₃ |
| S | 3-Cl, 4-Cl | Et | CH | CH₂CF₃ |
| S | 3-Cl, 5-Cl | Et | CH | CH₂CF₃ |
| S | 3-Cl, 4-Cl | i-Pr | CH | CH₂CF₃ |
| S | 3-Cl, 5-Cl | i-Pr | CH | CH₂CF₃ |
| S | 3-Cl, 4-Cl | Me | CH | CH₂CF₃ |
| S | 3-Cl, 5-Cl | Me | CH | CH₂CF₃ |
| O | 3-Cl, 4-Cl | OMe | CH | CH₂CF₃ |
| O | 3-Cl, 5-Cl | OMe | CH | CH₂CF₃ |
| S | 3-Cl, 4-Cl | Ph | CH | CH₂CF₃ |
| S | 3-Cl, 5-Cl | Ph | CH | CH₂CF₃ |
| O | 3-Cl, 4-Cl | SMe | CH | CH₂CF₃ |
| O | 3-Cl, 5-Cl | SMe | CH | CH₂CF₃ |
| O | 3-Cl, 4-Cl | Et | N | CH₂CF₃ |
| O | 3-Cl, 5-Cl | Et | N | CH₂CF₃ |
| S | 3-Cl, 4-Cl | Et | N | CH₂CF₃ |
| S | 3-Cl, 5-Cl | Et | N | CH₂CF₃ |
| O | 3-Cl, 4-Cl | i-Pr | N | CH₂CF₃ |
| O | 3-Cl, 5-Cl | i-Pr | N | CH₂CF₃ |
| S | 3-Cl, 4-Cl | i-Pr | N | CH₂CF₃ |
| S | 3-Cl, 5-Cl | i-Pr | N | CH₂CF₃ |
| O | 3-Cl, 4-Cl | Me | N | CH₂CF₃ |
| O | 3-Cl, 5-Cl | Me | N | CH₂CF₃ |
| S | 3-Cl, 4-Cl | Me | N | CH₂CF₃ |
| S | 3-Cl, 5-Cl | Me | N | CH₂CF₃ |
| O | 3-Cl, 4-Cl | Ph | N | CH₂CF₃ |
| O | 3-Cl, 5-Cl | Ph | N | CH₂CF₃ |
| S | 3-Cl, 4-Cl | Ph | N | CH₂CF₃ |
| S | 3-Cl, 5-Cl | Ph | N | CH₂CF₃ |

TABLE 3

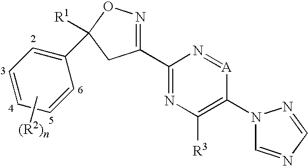

| R¹ | (R²)ₙ | R³ | A |
|---|---|---|---|
| CF₃ | 2-Cl | Et | CH |
| CF₃ | 2-Cl | Et | N |
| CF₃ | 2-Cl | i-Pr | CH |
| CF₃ | 2-Cl | i-Pr | N |
| CF₃ | 2-Cl | Me | CH |
| CF₃ | 2-Cl | Me | N |
| CF₃ | 2-Cl | Me | C—CN |
| CF₃ | 2-Cl | Me | C-Me |
| CF₃ | 2-Cl | Me | C—Br |
| CF₃ | 2-Cl | Me | C—Cl |
| CF₃ | 2-Cl | Ph | CH |
| CF₃ | 2-Cl | Ph | C—CN |
| Me | 2-Cl | Me | CH |
| Me | 2-Cl | Me | N |
| CF₃ | 2-Cl, 4-Cl | Et | CH |
| CF₃ | 2-Cl, 4-Cl | Et | C-Me |
| CF₃ | 2-Cl, 4-Cl | i-Pr | CH |
| CF₃ | 2-Cl, 4-Cl | i-Pr | C—Br |
| CF₃ | 2-Cl, 4-Cl | Me | CH |
| CF₃ | 2-Cl, 4-Cl | Me | N |
| CF₃ | 2-Cl, 4-Cl | Me | C—CN |
| CF₃ | 2-Cl, 4-Cl | Me | C-Me |
| CF₃ | 2-Cl, 4-Cl | Me | C—Br |
| CF₃ | 2-Cl, 4-Cl | Me | C—Cl |
| CF₃ | 2-Cl, 4-Cl | Ph | CH |
| CF₃ | 2-Cl, 4-Cl | Ph | C—CN |
| Me | 2-Cl, 4-Cl | Me | CH |
| Me | 2-Cl, 4-Cl | Me | N |
| CF₃ | 2-F | Et | CH |
| CF₃ | 2-F | Et | C-Me |
| CF₃ | 2-F | i-Pr | CH |
| CF₃ | 2-F | i-Pr | C—Br |
| CF₃ | 2-F | Me | CH |
| CF₃ | 2-F | Me | N |
| CF₃ | 2-F | Me | C—CN |
| CF₃ | 2-F | Me | C-Me |
| CF₃ | 2-F | Me | C—Br |
| CF₃ | 2-F | Me | C—Cl |
| CF₃ | 2-F | Ph | CH |
| CF₃ | 2-F | Ph | N |
| Me | 2-F | Me | CH |
| Me | 2-F | Me | N |
| CF₃ | 2-F, 4-F | Et | CH |
| CF₃ | 2-F, 4-F | Et | C-Me |
| CF₃ | 2-F, 4-F | i-Pr | CH |
| CF₃ | 2-F, 4-F | i-Pr | C—Cl |
| CF₃ | 2-F, 4-F | Me | CH |
| CF₃ | 2-F, 4-F | Me | N |
| CF₃ | 2-F, 4-F | Me | C—CN |
| CF₃ | 2-F, 4-F | Me | C-Me |
| CF₃ | 2-F, 4-F | Me | C—Br |
| CF₃ | 2-F, 4-F | Me | C—Cl |
| CF₃ | 2-F, 4-F | Ph | CH |
| CF₃ | 2-F, 4-F | Ph | C-Me |
| Me | 2-F, 4-F | Me | CH |
| Me | 2-F, 4-F | Me | N |
| CF₃ | 3-CF₃ | Et | CH |
| CF₃ | 3-CF₃ | Et | C—Cl |
| CF₃ | 3-CF₃ | i-Pr | CH |
| CF₃ | 3-CF₃ | i-Pr | C—CN |
| CF₃ | 3-CF₃ | Me | CH |
| CF₃ | 3-CF₃ | Me | N |
| CF₃ | 3-CF₃ | Me | C—CN |
| CF₃ | 3-CF₃ | Me | C-Me |
| CF₃ | 3-CF₃ | Me | C—Br |
| CF₃ | 3-CF₃ | Me | C—Cl |
| CF₃ | 3-CF₃ | Ph | CH |
| CF₃ | 3-CF₃ | Ph | C-Me |
| Me | 3-CF₃ | Me | CH |
| Me | 3-CF₃ | Me | N |
| CF₃ | 3-Br | Et | CH |
| CF₃ | 3-Br | Et | N |
| CF₃ | 3-Br | i-Pr | CH |
| CF₃ | 3-Br | i-Pr | C—Cl |
| CF₃ | 3-Br | i-Pr | CH |
| CF₃ | 3-Br | Me | N |
| CF₃ | 3-Br | Me | C—CN |
| CF₃ | 3-Br | Me | C-Me |
| CF₃ | 3-Br | Me | C—Br |
| CF₃ | 3-Br | Me | C—Cl |
| CF₃ | 3-Br | Ph | CH |
| CF₃ | 3-Br | Ph | C-Me |
| Me | 3-Br | Me | CH |
| Me | 3-Br | Me | N |
| CF₃ | 3-CF₃, 5-CF₃ | Et | N |
| CF₃ | 3-CF₃. 5-CF₃ | Et | CH |
| CF₃ | 3-CF₃, 5-CF₃ | i-Pr | CH |
| CF₃ | 3-CF₃, 5-CF₃ | i-Pr | C-Me |
| CF₃ | 3-CF₃, 5-CF₃ | Me | CH |
| CF₃ | 3-CF₃, 5-CF₃ | Me | N |
| CF₃ | 3-CF₃, 5-CF₃ | Me | C—CN |
| CF₃ | 3-CF₃, 5-CF₃ | Me | C-Me |
| CF₃ | 3-CF₃, 5-CF₃ | Me | C—Br |
| CF₃ | 3-CF₃, 5-CF₃ | Me | C—Cl |
| CF₃ | 3-CF₃, 5-CF₃ | Ph | CH |
| CF₃ | 3-CF₃, 5-CF₃ | Ph | C-Me |
| Me | 3-CF₃, 5-CF₃ | Me | CH |
| Me | 3-CF₃, 5-CF₃ | Me | N |
| CF₃ | 3-Cl | Et | CH |
| CF₃ | 3-Cl | Et | N |
| CF₃ | 3-Cl | i-Pr | CH |
| CF₃ | 3-Cl | i-Pr | C—Br |
| CF₃ | 3-Cl | Me | CH |
| CF₃ | 3-Cl | Me | N |
| CF₃ | 3-Cl | Me | C—CN |
| CF₃ | 3-Cl | Me | C-Me |
| CF₃ | 3-Cl | Me | C—Br |
| CF₃ | 3-Cl | Me | C—Cl |
| CF₃ | 3-Cl | Ph | CH |
| CF₃ | 3-Cl | Ph | N |
| Me | 3-Cl | Me | CH |
| Me | 3-Cl | Me | C—Cl |
| CF₃ | 3-Cl, 4-Cl | 2,4-di-F-Ph | CH |
| CF₃ | 3-Cl, 4-Cl | CF₃ | C-Me |
| CF₃ | 3-Cl, 4-Cl | CF₃ | CH |
| CF₃ | 3-Cl, 4-Cl | CN | CH |
| CF₃ | 3-Cl, 4-Cl | Et | CH |
| CF₃ | 3-Cl, 4-Cl | Et | C—Br |
| CF₃ | 3-Cl, 4-Cl | Et | N |
| CF₃ | 3-Cl, 4-Cl | i-Pr | CH |
| CF₃ | 3-Cl, 4-Cl | i-Pr | N |
| CF₃ | 3-Cl, 4-Cl | i-Pr | C-Me |
| CF₃ | 3-Cl, 4-Cl | Me | CH |
| CF₃ | 3-Cl, 4-Cl | Me | N |
| CF₃ | 3-Cl, 4-Cl | Me | C—CN |
| CF₃ | 3-Cl, 4-Cl | Me | C-Me |
| CF₃ | 3-Cl, 4-Cl | Me | C—Br |
| CF₃ | 3-Cl, 4-Cl | Me | C—Cl |
| CF₃ | 3-Cl, 4-Cl | NO₂ | CH |
| CF₃ | 3-Cl, 4-Cl | n-Pr | CH |
| CF₃ | 3-Cl, 4-Cl | Ph | CH |
| CF₃ | 3-Cl, 4-Cl | Ph | C—Cl |
| CF₃ | 3-Cl, 4-Cl | Ph | N |
| CF₃ | 3-Cl, 4-Cl | t-Bu | CH |
| Me | 3-Cl, 4-Cl | Me | CH |
| Me | 3-Cl, 4-Cl | Me | N |
| CF₃ | 3-Cl, 5-Cl | 2,4-di-F-Ph | CH |
| CF₃ | 3-Cl, 5-Cl | CF₃ | C-Me |
| CF₃ | 3-Cl, 5-Cl | CF₃ | CH |
| CF₃ | 3-Cl, 5-Cl | CN | CH |
| CF₃ | 3-Cl, 5-Cl | Et | CH |
| CF₃ | 3-Cl, 5-Cl | Et | C—Br |
| CF₃ | 3-Cl, 5-Cl | Et | C-Me |
| CF₃ | 3-Cl, 5-Cl | i-Pr | CH |
| CF₃ | 3-Cl, 5-Cl | i-Pr | N |
| CF₃ | 3-Cl, 5-Cl | i-Pr | C-Me |
| CF₃ | 3-Cl, 5-Cl | Me | CH |
| CF₃ | 3-Cl, 5-Cl | Me | N |
| CF₃ | 3-Cl, 5-Cl | Me | C—CN |
| CF₃ | 3-Cl, 5-Cl | Me | C-Me |
| CF₃ | 3-Cl, 5-Cl | Me | C—Br |
| CF₃ | 3-Cl, 5-Cl | Me | C—Cl |
| CF₃ | 3-Cl, 5-Cl | NO₂ | CH |
| CF₃ | 3-Cl, 5-Cl | n-Pr | CH |
| CF₃ | 3-Cl, 5-Cl | Ph | CH |
| CF₃ | 3-Cl, 5-Cl | Ph | C—Br |
| CF₃ | 3-Cl, 5-Cl | Ph | N |
| CF₃ | 3-Cl, 5-Cl | t-Bu | CH |
| Me | 3-Cl, 5-Cl | Me | CH |
| Me | 3-Cl, 5-Cl | Me | N |
| CF₃ | 3-CN | Et | CH |
| CF₃ | 3-CN | Et | C-Me |
| CF₃ | 3-CN | i-Pr | CH |
| CF₃ | 3-CN | i-Pr | N |
| CF₃ | 3-CN | Me | CH |
| CF₃ | 3-CN | Me | N |
| CF₃ | 3-CN | Me | C—CN |
| CF₃ | 3-CN | Me | C-Me |
| CF₃ | 3-CN | Me | C—Br |
| CF₃ | 3-CN | Me | C—Cl |
| CF₃ | 3-CN | Ph | CH |
| CF₃ | 3-CN | Ph | N |
| Me | 3-CN | Me | CH |
| Me | 3-CN | Me | N |
| CF₃ | 3-F | Et | C—CN |
| CF₃ | 3-F | Et | N |
| CF₃ | 3-F | i-Pr | C-Me |
| CF₃ | 3-F | i-Pr | N |
| CF₃ | 3-F | Me | CH |
| CF₃ | 3-F | Me | N |
| CF₃ | 3-F | Me | C—CN |
| CF₃ | 3-F | Me | C-Me |
| CF₃ | 3-F | Me | C—Br |
| CF₃ | 3-F | Me | C—Cl |
| CF₃ | 3-F | Ph | CH |
| CF₃ | 3-F | Ph | N |
| Me | 3-F | Me | CH |
| Me | 3-F | Me | N |
| CF₃ | 3-F, 4-F | Et | CH |
| CF₃ | 3-F, 4-F | Et | N |
| CF₃ | 3-F, 4-F | i-Pr | CH |
| CF₃ | 3-F, 4-F | i-Pr | N |
| CF₃ | 3-F, 4-F | Me | CH |
| CF₃ | 3-F, 4-F | Me | N |
| CF₃ | 3-F, 4-F | Me | C—CN |
| CF₃ | 3-F, 4-F | Me | C-Me |
| CF₃ | 3-F, 4-F | Me | C—Br |
| CF₃ | 3-F, 4-F | Me | C—Cl |
| CF₃ | 3-F, 4-F | Ph | CH |
| CF₃ | 3-F, 4-F | Ph | N |
| Me | 3-F, 4-F | Me | CH |
| Me | 3-F, 4-F | Me | N |
| CF₃ | 3-F, 5-F | Et | CH |
| CF₃ | 3-F, 5-F | Et | N |
| CF₃ | 3-F, 5-F | i-Pr | CH |
| CF₃ | 3-F, 5-F | i-Pr | N |
| CF₃ | 3-F, 5-F | Me | CH |
| CF₃ | 3-F, 5-F | Me | N |
| CF₃ | 3-F, 5-F | Me | C—CN |
| CF₃ | 3-F, 5-F | Me | C-Me |
| CF₃ | 3-F, 5-F | Me | C—Br |
| CF₃ | 3-F, 5-F | Me | C—Cl |
| CF₃ | 3-F, 5-F | Me | C-Me |
| CF₃ | 3-F, 5-F | Ph | C—CN |
| CF₃ | 3-F, 5-F | Ph | N |
| Me | 3-F, 5-F | Me | CH |
| CF₃ | 3-I | Et | CH |
| CF₃ | 3-I | Et | N |
| CF₃ | 3-I | i-Pr | CH |
| CF₃ | 3-I | i-Pr | C—Cl |
| CF₃ | 3-I | Me | CH |
| CF₃ | 3-I | Me | N |
| CF₃ | 3-I | Me | C—CN |
| CF₃ | 3-I | Me | C-Me |
| CF₃ | 3-I | Me | C—Br |
| CF₃ | 3-I | Me | C—Cl |
| CF₃ | 3-I | Ph | CH |
| CF₃ | 3-I | Ph | N |
| Me | 3-I | Me | CH |
| Me | 3-I | Me | N |
| CF₃ | 3-Me | Et | CH |
| CF₃ | 3-Me | Et | N |
| CF₃ | 3-Me | i-Pr | CH |
| CF₃ | 3-Me | i-Pr | N |
| CF₃ | 3-Me | Me | CH |
| CF₃ | 3-Me | Me | N |
| CF₃ | 3-Me | Me | C—CN |
| CF₃ | 3-Me | Me | C-Me |
| CF₃ | 3-Me | Me | C—Br |
| CF₃ | 3-Me | Me | C—Cl |
| CF₃ | 3-Me | Ph | C-Me |
| CF₃ | 3-Me | Ph | N |
| Me | 3-Me | Me | CH |
| Me | 3-Me | Me | N |
| CF₃ | 3-OCF₃ | Et | C—CN |
| CF₃ | 3-OCF₃ | Et | N |
| CF₃ | 3-OCF₃ | i-Pr | C-Me |
| CF₃ | 3-OCF₃ | i-Pr | CH |
| CF₃ | 3-OCF₃ | Me | CH |
| CF₃ | 3-OCF₃ | Me | N |
| CF₃ | 3-OCF₃ | Me | C—CN |
| CF₃ | 3-OCF₃ | Me | C-Me |
| CF₃ | 3-OCF₃ | Me | C—Br |
| CF₃ | 3-OCF₃ | Me | C—Cl |
| CF₃ | 3-OCF₃ | Ph | CH |
| CF₃ | 3-OCF₃ | Ph | C—Br |
| Me | 3-OCF₃ | Me | CH |
| Me | 3-OCF₃ | Me | C—Cl |
| CF₃ | 3-OMe | Et | CH |
| CF₃ | 3-OMe | Et | N |
| CF₃ | 3-OMe | i-Pr | CH |
| CF₃ | 3-OMe | i-Pr | C-Me |
| CF₃ | 3-OMe | Me | CH |
| CF₃ | 3-OMe | Me | N |
| CF₃ | 3-OMe | Me | C—CN |
| CF₃ | 3-OMe | Me | C-Me |
| CF₃ | 3-OMe | Me | C—Br |
| CF₃ | 3-OMe | Me | C—Cl |
| CF₃ | 3-OMe | Ph | CH |
| CF₃ | 3-OMe | Ph | C—Br |
| Me | 3-OMe | Me | CH |
| Me | 3-OMe | Me | C—Cl |
| CF₃ | 4-CF₃ | Et | CH |
| CF₃ | 4-CF₃ | Et | N |
| CF₃ | 4-CF₃ | i-Pr | CH |
| CF₃ | 4-CF₃ | i-Pr | C-Me |
| CF₃ | 4-CF₃ | Me | CH |
| CF₃ | 4-CF₃ | Me | N |
| CF₃ | 4-CF₃ | Me | C—CN |
| CF₃ | 4-CF₃ | Me | C-Me |
| CF₃ | 4-CF₃ | Me | C—Br |
| CF₃ | 4-CF₃ | Me | C—Cl |
| CF₃ | 4-CF₃ | Ph | CH |
| CF₃ | 4-CF₃ | Ph | N |
| Me | 4-CF₃ | Me | CH |
| Me | 4-CF₃ | Me | C—Cl |
| CF₃ | 4-Br | Et | CH |
| CF₃ | 4-Br | Et | N |
| CF₃ | 4-Br | i-Pr | CH |
| CF₃ | 4-Br | i-Pr | C—CN |
| CF₃ | 4-Br | Me | CH |
| CF₃ | 4-Br | Me | N |
| CF₃ | 4-Br | Me | C—CN |
| CF₃ | 4-Br | Me | C-Me |
| CF₃ | 4-Br | Me | C—Br |
| CF₃ | 4-Br | Me | C—Cl |
| CF₃ | 4-Br | Ph | CH |
| CF₃ | 4-Br | Ph | C-Me |
| Me | 4-Br | Me | CH |
| CF₃ | 4-Cl | Et | C—Br |
| CF₃ | 4-Cl | Et | C—Cl |
| CF₃ | 4-Cl | i-Pr | CH |
| CF₃ | 4-Cl | i-Pr | N |
| CF₃ | 4-Cl | Me | CH |
| CF₃ | 4-Cl | Me | N |
| CF₃ | 4-Cl | Me | C—CN |
| CF₃ | 4-Cl | Me | C-Me |
| CF₃ | 4-Cl | Me | C—Br |
| CF₃ | 4-Cl | Me | C—Cl |
| CF₃ | 4-Cl | Ph | CH |
| CF₃ | 4-Cl | Ph | C-Me |
| Me | 4-Cl | Me | CH |
| Me | 4-Cl | Me | N |
| CF₃ | 4-CN | Et | C—Br |
| CF₃ | 4-CN | Et | C—Cl |
| CF₃ | 4-CN | i-Pr | CH |
| CF₃ | 4-CN | i-Pr | N |
| CF₃ | 4-CN | Me | CH |
| CF₃ | 4-CN | Me | N |
| CF₃ | 4-CN | Me | C-Me |
| CF₃ | 4-CN | Me | C—Br |
| CF₃ | 4-CN | Me | C—Cl |
| CF₃ | 4-CN | Ph | C—CN |
| CF₃ | 4-CN | Ph | C-Me |
| Me | 4-CN | Me | CH |
| Me | 4-CN | Me | N |
| CF₃ | 4-F | Et | C—Br |
| CF₃ | 4-F | Et | CH |
| CF₃ | 4-F | i-Pr | N |
| CF₃ | 4-F | i-Pr | C-Me |
| CF₃ | 4-F | Me | CH |
| CF₃ | 4-F | Me | N |
| CF₃ | 4-F | Me | C—CN |
| CF₃ | 4-F | Me | C-Me |
| CF₃ | 4-F | Me | C—Br |
| CF₃ | 4-F | Me | C—Cl |
| CF₃ | 4-F | Ph | CH |
| CF₃ | 4-F | Ph | C-Me |
| Me | 4-F | Me | CH |
| Me | 4-F | Me | N |
| CF₃ | 4-I | Et | CH |
| CF₃ | 4-I | Et | N |
| CF₃ | 4-I | i-Pr | CH |
| CF₃ | 4-I | i-Pr | N |
| CF₃ | 4-I | Me | CH |
| CF₃ | 4-I | Me | N |
| CF₃ | 4-I | Me | C—CN |
| CF₃ | 4-I | Me | C-Me |
| CF₃ | 4-I | Me | C—Br |
| CF₃ | 4-I | Me | C—Cl |
| CF₃ | 4-I | Ph | C-Me |
| CF₃ | 4-I | Ph | N |
| Me | 4-I | Me | CH |
| Me | 4-I | Me | N |
| CF₃ | 4-Me | Et | CH |
| CF₃ | 4-Me | Et | N |
| CF₃ | 4-Me | i-Pr | C—Br |
| CF₃ | 4-Me | i-Pr | CH |
| CF₃ | 4-Me | Me | CH |
| CF₃ | 4-Me | Me | N |
| CF₃ | 4-Me | Me | C—CN |
| CF₃ | 4-Me | Me | C-Me |
| CF₃ | 4-Me | Me | C—Br |
| CF₃ | 4-Me | Me | C—Cl |
| CF₃ | 4-Me | Ph | CH |
| CF₃ | 4-Me | Ph | C-Me |
| Me | 4-Me | Me | CH |
| Me | 4-Me | Me | N |
| CF₃ | 4-OCF₃ | Et | CH |
| CF₃ | 4-OCF₃ | Et | C—Br |
| CF₃ | 4-OCF₃ | i-Pr | CH |
| CF₃ | 4-OCF₃ | i-Pr | N |
| CF₃ | 4-OCF₃ | Me | CH |
| CF₃ | 4-OCF₃ | Me | N |
| CF₃ | 4-OCF₃ | Me | C—CN |
| CF₃ | 4-OCF₃ | Me | C-Me |
| CF₃ | 4-OCF₃ | Me | C—Br |
| CF₃ | 4-OCF₃ | Me | C—Cl |
| CF₃ | 4-OCF₃ | Ph | CH |
| CF₃ | 4-OCF₃ | Ph | C—CN |
| Me | 4-OCF₃ | Me | CH |
| Me | 4-OCF₃ | Me | N |
| Me | 4-OCF₃ | Me | C-Me |
| CF₃ | 4-OMe | Et | CH |
| CF₃ | 4-OMe | Et | N |
| CF₃ | 4-OMe | i-Pr | CH |
| CF₃ | 4-OMe | i-Pr | N |
| CF₃ | 4-OMe | Me | CH |
| CF₃ | 4-OMe | Me | N |
| CF₃ | 4-OMe | Me | C—CN |
| CF₃ | 4-OMe | Me | C-Me |
| CF₃ | 4-OMe | Me | C—Br |
| CF₃ | 4-OMe | Me | C—Cl |
| CF₃ | 4-OMe | Ph | C-Me |
| CF₃ | 4-OMe | Ph | CH |
| Me | 4-OMe | Me | CH |
| Me | 4-OMe | Me | N |
| CF₃ | H | Et | C—Br |
| CF₃ | H | Et | CH |
| CF₃ | H | i-Pr | C—Br |
| CF₃ | H | i-Pr | CH |
| CF₃ | H | Me | CH |
| CF₃ | H | Me | N |
| CF₃ | H | Me | C—CN |
| CF₃ | H | Me | C-Me |
| CF₃ | H | Me | C—Br |
| CF₃ | H | Me | C—Cl |
| CF₃ | H | Ph | N |
| CF₃ | H | Ph | C-Me |
| Me | H | Me | C—CN |
| Me | H | Me | CH |

### Formulation/Utility

A compound of this invention will generally be used as an invertebrate pest control active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active Ingredient | Diluent | Surfactant |
| Water-Dispersible and Water- | 0.001-90 | 0-99.999 | 0-15 |
| soluble Granules, Tablets | | | |
| and Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkypyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g, oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylene-tetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 14748, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 14; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Tables A-B. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### EXAMPLE A

Wettable Powder

| | |
|---|---|
| Compound 7 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### EXAMPLE B

Granule

| | |
|---|---|
| Compound 10 | 10.0% |
| attapulgite granules (low volatile matter, 0.71/0.30 mm; | 90.0% |
| U.S.S. No. 25-50 sieves) | |

### EXAMPLE C

Extruded Pellet

| | |
|---|---|
| Compound 3 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### EXAMPLE D

Emulsifiable Concentrate

| | |
|---|---|
| Compound 1 | 10.0% |
| polyoxyethylene sorbitol hexoleate | 20.0% |
| C₆-C₁₀ fatty acid methyl ester | 70.0% |

### EXAMPLE E

Microemulsion

| | |
|---|---|
| Compound 2 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### EXAMPLE F

Seed Treatment

| | |
|---|---|
| Compound 7 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### EXAMPLE G

High Strength Concentrate

| | |
|---|---|
| Compound 1 | 98.5% |
| silica aerogel | 0.5% |
| synthetic amorphous fine silica | 1.0% |

### EXAMPLE H

Fertilizer Stick

| | |
|---|---|
| Compound 7 | 2.5% |
| pyrrolidone-styrene copolymer | 4.8% |
| tristyrylphenyl 16-ethoxylate | 2.3% |
| talc | 0.8% |
| corn starch | 5.0% |
| Nitrophoska ® Permanent 15-9-15 slow- | 36.0% |
| release fertilizer (BASF) | |
| kaolin | 38.0% |
| water | 10.6% |

Compounds of this invention exhibit activity against a wide spectrum of invertebrate pests. These pests include invertebrates inhabiting a variety of environments such as, for example, plant foliage, roots, soil, harvested crops or other foodstuffs, building structures or animal integuments. These pests include, for example, invertebrates feeding on foliage (including leaves, stems, flowers and fruits), seeds, wood, textile fibers or animal blood or tissues, and thereby causing injury or damage to, for example, growing or stored agronomic crops, forests, greenhouse crops, ornamentals, nursery crops, stored foodstuffs or fiber products, or houses or other structures or their contents, or being harmful to animal health or public health. Those skilled in the art will appreciate that not all compounds are equally effective against all growth stages of all pests.

These present compounds and compositions are thus useful agronomically for protecting field crops from phytophagous invertebrate pests, and also nonagronomically for protecting other horticultural crops and plants from phytophagous invertebrate pests. This utility includes protecting crops and other plants (i.e. both agronomic and nonagronomic) that contain genetic material introduced by genetic engineering (i.e. transgenic) or modified by mutagenesis to provide advantageous traits. Examples of such traits include tolerance to herbicides, resistance to phytophagous pests (e.g., insects, mites, aphids, spiders, nematodes, snails, plant-pathogenic fungi, bacteria and viruses), improved plant growth, increased tolerance of adverse growing conditions such as high or low temperatures, low or high soil moisture, and high salinity, increased flowering or fruiting, greater harvest yields, more rapid maturation, higher quality and/or nutritional value of the harvested product, or improved storage or process properties of the harvested products. Transgenic plants can be modified to express multiple traits. Examples of plants containing traits provided by genetic engineering or mutagenesis include varieties of corn, cotton, soybean and potato expressing an insecticidal Bacillus thuringiensis toxin such as YIELD GARD®, KNOCKOUT®, STARLINK®, BOLLGARD®, NuCOTN® and NEWLEAF®, and herbicide-tolerant varieties of corn, cotton, soybean and rapeseed such as ROUNDUP READY®, LIBERTY LINK®, IMI®, STS® and CLEARFIELD®, as well as crops expressing N-acetyltransferase (GAT) to provide resistance to glyphosate herbicide, or crops containing the HRA gene providing resistance to herbicides inhibiting acetolactate synthase (ALS). The present compounds and compositions may interact synergistically with traits introduced by genetic engineering or modified by mutagenesis, thus enhancing phenotypic expression or effectiveness of the traits or increasing the invertebrate pest control effectiveness of the present compounds and compositions. In particular, the present compounds and compositions may interact synergistically with the phenotypic expression of proteins or other natural products toxic to invertebrate pests to provide greater-than-additive control of these pests.

Nonagronomic uses refer to invertebrate pest control in the areas other than fields of crop plants. Nonagronomic uses of the present compounds and compositions include control of invertebrate pests in stored grains, beans and other foodstuffs, and in textiles such as clothing and carpets. Nonagronomic uses of the present compounds and compositions also include invertebrate pest control in ornamental plants, forests, in yards, along roadsides and railroad rights of way, and on turf such as lawns, golf courses and pastures. Nonagronomic uses of the present compounds and compositions also include invertebrate pest control in houses and other buildings which may be occupied by humans and/or companion, farm, ranch, zoo or other animals. Nonagronomic uses of the present compounds and compositions also include the control of pests such as termites that can damage wood or other structural materials used in buildings.

Nonagronomic uses of the present compounds and compositions also include protecting human and animal health by controlling invertebrate pests that are parasitic or transmit infectious diseases. The controlling of animal parasites includes controlling external parasites that are parasitic to the surface of the body of the host animal (e.g., shoulders, armpits, abdomen, inner part of the thighs) and internal parasites that are parasitic to the inside of the body of the host animal (e.g., stomach, intestine, lung, veins, under the skin, lymphatic tissue). External parasitic or disease transmitting pests include, for example, chiggers, ticks, lice, mosquitoes, flies, mites and fleas. Internal parasites include heartworms, hookworms and helninths. Compounds and compositions of the present invention are suitable for systemic and/or non-systemic control of infestation or infection by parasites on animals. Compounds and compositions of the present invention are particularly suitable for combating external parasitic or disease transmitting pests. Compounds and compositions of the present invention are suitable for combating parasites that infest agricultural working animals, such as cattle, sheep, goats, horses, pigs, donkeys, camels, buffalos, rabbits, hens, turkeys, ducks, geese and bees; pet animals and domestic animals such as dogs, cats, pet birds and aquarium fish; as well as so-called experimental animals, such as hamsters, guinea pigs, rats and mice. By combating these parasites, fatalities and performance reduction (in terms of meat, milk, wool, skins, eggs, honey, etc.) are reduced, so that applying a composition comprising a compound of the present invention allows more economic and simple husbandry of animals.

Examples of agronomic or nonagronomic invertebrate pests include eggs, larvae and adults of the order Lepidoptera, such as armyworms, cutworms, loopers, and heliothines in the family Noctuidae (e.g., pink stem borer (Sesamia inferens Walker), corn stalk borer (Sesamia nonagrioides Lefebvre), southern armyworm (Spodoptera eridania Cramer), fall arnyworm (Spodoptera fugiperda J. E. Smith), beet armyworm (Spodoptera exigua Hübner), cotton leafworm (Spodoptera littoralis Boisduval), yellowstriped armyworm (Spodoptera ornithogalli Guenée), black cutworm (Agrotis ipsilon Hufnagel), velvetbean caterpillar (Anticarsia gemmatalis Hübner), green fruitworm (Lithophane antennata Walker), cabbage armyworm (Barathra brassicae Linnaeus), soybean looper (Pseudoplusia includens Walker), cabbage looper (Trichoplusia ni Hübner), tobacco budworm (Heliothis virescens Fabricius)); borers, casebearers, webworms, coneworms, cabbageworms and skeletonizers from the family Pyralidae (e.g., European corn borer (Ostrinia nubilalis Hübner), navel orangeworm (Amyelois transitella Walker), corn root webworm (Crambus caliginosellus Clemens), sod webworms (Pyralidae: Crambinae) such as sod worm (Herpetogramma licarsisalis Walker), sugarcane stem borer (Chilo infuscatellus Snellen), tomato small borer (Neoleucinodes elegantalis Guenée), green leafroller (Cnaphalocerus medinalis), grape leaffolder (Desmia funeralis Hübner), melon worm (Diaphania nitidalis Stoll), cabbage center grub (Helluala hydralis Guenée), yellow stem borer (Scirpophaga incertulas Walker), early shoot borer (Scirpophaga infuscatellus Snellen), white stem borer (Scirpophaga innotata Walker), top shoot borer (Scirpophaga nivella Fabricius), dark-headed rice borer (Chilo polychrysus Meyrick), cabbage cluster caterpillar (Crocidolomia binotalis English)); leafrollers, budworms, seed worms, and fruit worms in the family Tortricidae (e.g., codling moth (Cydia pomonella Linnaeus), grape berry moth (Endopiza viteana Clemens), oriental fruit moth (Grapholita molesta Busck), citrus false codling moth (Cryptophlebia leucotreta Meyrick), citrus borer (Ecdytolopha aurantiana Lima), redbanded leafroller (Argyrotaenia velutinana Walker), obliquebanded leafroller (Choristoneura rosaceana Harris), light brown apple moth (Epiphyas postvittana Walker), European grape berry moth (Eupoecilia ambiguella Hübner), apple bud moth (Pandemis pyrusana Kearfott), omnivorous leafroller (Platynota stultana Walsingham), barred fruit-tree tortrix (Pandemis cerasana Hübner), apple brown tortrix (Pandemis heparana Denis & Schiffermüller)); and many other economically important lepidoptera(e.g., diamondback moth (Plutella xylostella Linnaeus), pink bollworm (Pectinophora gossypiella Saunders), gypsy moth (Lymantria dispar Linnaeus), peach fruit borer (Carposina niponensis Walsingham), peach twig borer (Anarsia lineatella Zeller), potato tuberworm (Phthorimaea operculella Zeller), spotted teniform leafminer (Lithocolletis blancardella Fabricius), Asiatic apple leafminer (Lithocolletis ringoniella Matsumura), rice leaffolder (Lerodea eufala Edwards), apple leafminer (Leucoptera scitella Zeller)); eggs, nymphs and adults of the order Blattodea including cockroaches from the families Blattellidae and Blattidae (e.g., oriental cockroach (Blatta orientalis Linnaeus), Asian cockroach (Blatella asahinai Mizukubo), German cockroach (Blattella germanica Linnaeus), brownbanded cockroach (Supella longipalpa Fabricius), American cockroach (Periplaneta americana Linnaeus), brown cockroach (Periplaneta brunnea Burmeister), Madeira cockroach (Leucophaea maderae Fabricius)), smoky brown cockroach (Periplaneta fuliginosa Service), Australian Cockroach (Periplaneta australasiae Fabr.), lobster cockroach (Nauphoeta cinerea Olivier) and smooth cockroach (Symploce pallens Stephens)); eggs, foliar feeding, fruit feeding, root feeding, seed feeding and vesicular tissue feeding larvae and adults of the order Coleoptera including weevils from the families Anthribidae, Bruchidae, and Curculionidae (e.g., boll weevil (Anthonomus grandis Boheman), rice water weevil (Lissorhoptrus oryzophilus Kuschel), granary weevil (Sitophilus granarius Linnaeus), rice weevil (Sitophilus oryzae Linnaeus)), annual bluegrass weevil (Listronotus maculicollis Dietz), bluegrass billbug (Sphenophorus parvulus Gyllenhal), hunting billbug (Sphenophorus venatus vestitus), Denver billbug (Sphenophorus cicatristriatus Fahraeus)); flea beetles, cucumber beetles, rootworms, leaf beetles, potato beetles, and leafminers in the family Chrysomelidae (e.g., Colorado potato beetle (Leptinotarsa decemlineata Say), western corn rootworm (Diabrotica virgifera virgifera LeConte)); chafers and other beetles from the family Scarabaeidae (e.g., Japanese beetle (Popillia japonica Newman), oriental beetle (Anomala orientalis Waterhouse, Exomala orientalis (Waterhouse) Baraud), northern masked chafer (Cyclocephala borealis Arrow), southern masked chafer (Cyclocephala immaculata Olivier or C. lurida Bland) dung beetle and white grub (Aphodius spp.), black turfgrass ataenius (Ataenius spretulus Haldeman), green June beetle (Cotinis nitida Linnaeus), Asiatic garden beetle (Maladera castanea Arrow), May/June beetles (Phyllophaga spp.) and European chafer (Rhizotrogus majalis Razoumowsky)); carpet beetles from the family Dermestidae; wireworms from the family Elateridae; bark beetles from the family Scolytidae and flour beetles from the family Tenebrionidae. In addition, agronomic and nonagronomic pests include: eggs, adults and larvae of the order Dermaptera including earwigs from the family Forficulidae (e.g., European earwig (Forficula auricularia Linnaeus), black earwig (Chelisoches morio Fabricius)); eggs, immatures, adults and nymphs of the orders Hemiptera and Homoptera such as, plant bugs from the family Miridae, cicadas from the family Cicadidae, leafhoppers (e.g. Empoasca spp.) from the family Cicadellidae, bed bugs (e.g., Cimex lectularius Linnaeus) from the family Cimicidae, planthoppers from the families Fulgoroidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Coccidae, Diaspididae and Margarodidae, lace bugs from the family Tingidae, stink bugs from the family Pentatomidae, chinch bugs (e.g., hairy chinch bug (Blissus leucopterus hirtus Montandon) and southern chinch bug (Blissus insularis Barber)) and other seed bugs from the family Lygaeidae, spittlebugs from the family Cercopidae squash bugs from the family Coreidae, and red bugs and cotton stainers from the family Pyrrhocoridae. Also included are eggs, larvae, nymphs and adults of the order Acari (mites) such as spider mites and red mites in the family Tetranychidae (e.g., European red mite (Panonychus ulmi Koch), two spotted spider mite (Tetranychus urticae Koch), McDaniel mite (Tetranychus mcdanieli McGregor)); flat mites in the family Tenuipalpidae (e.g., citrus flat mite (Brevipalpus lewisi McGregor)); rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae (e.g., deer tick (Ixodes scapularis Say), Australian paralysis tick (Ixodes holocyclus Neumann), American dog tick (Dermacentor variabilis Say), lone star tick (Amblyomma americanum Linnaeus)) and scab and itch mites in the families Psoroptidae, Pyemotidae, and Sarcoptidae; eggs, adults and immatures of the order Orthoptera including grasshoppers, locusts and crickets (e.g., migratory grasshoppers (e.g., Melanoplus sanguinipes Fabricius, M. differentialis Thomas), American grasshoppers (e.g., Schistocerca americana Drury), desert locust (Schistocerca gregaria Forskal), migratory locust (Locusta migratoria Linnaeus), bush locust (Zonocerus spp.), house cricket (Acheta domesticus Linnaeus), mole crickets (e.g., tawny mole cricket (Scapteriscus vicinus Scudder) and southern mole cricket (Scapteriscus borellii Giglio-Tos)); eggs, adults and immatures of the order Diptera including leafminers (e.g., Liriomyza spp. such as serpentine vegetable leafminer (Liriomyza sativae Blanchard)), midges, fruit flies (Tephritidae), frit flies (e.g., Oscinella frit Linnaeus), soil maggots, house flies (e.g., Musca domestica Linnaeus), lesser house flies (e.g., Fannia canicularis Linnaeus, F. femoralis Stein), stable flies (e.g., Stomoxys calcitrans Linnaeus), face flies, horn flies, blow flies (e.g., Chrysomya spp., Phormia spp.), and other muscoid fly pests, horse flies (e.g., Tabanus spp.), bot flies (e.g., Gastrophilus spp., Oestrus spp.), cattle grubs (e.g., Hypoderma spp.), deer flies (e.g., Chrysops spp.), keds (e.g., Melophagus ovinus Linnaeus) and other Brachycera, mosquitoes (e.g., Aedes spp., Anopheles spp., Culex spp.), black flies (e.g., Prosimulium spp., Simulium spp.), biting midges, sand flies, sciarids, and other Nematocera; eggs, adults and immatures of the order Thysanoptera including onion thrips (Thrips tabaci Lindeman), flower thrips (Frankliniella spp.), and other foliar feeding thrips; insect pests of the order Hymenoptera including ants of the Family Formicidae including the Florida carpenter ant (Camponotus floridanus Buckley), red carpenter ant (Camponotus ferrugineus Fabricius), black carpenter ant (Camponotus pennsylvanicus De Geer), white-footed ant (Technomyrmex albipes fr. Smith), big headed ants (Pheidole sp.), ghost ant (Tapinoma melanocephalum Fabricius); Pharaoh ant (Monomorium pharaonis Linnaeus), little fire ant (Wasmannia auropunctata Roger), fire ant (Solenopsis geminata Fabricius), red imported fire ant (Solenopsis invicta Buren), Argentine ant (Iridomyrmex humilis Mayr), crazy ant (Paratrechina longicornis Latreille), pavement ant (Tetramorium caespitum Linnaeus), cornfield ant (Lasius alienus Forster) and odorous house ant (Tapinoma sessile Say). Other Hymenoptera including bees (including carpenter bees), hornets, yellow jackets, wasps, and sawflies (Neodiprion spp.; Cephus spp.); insect pests of the order Isoptera including termites in the Termitidae (e.g., Macrotermes sp., Odontotermes obesus Rambur), Kalotermitidae (e.g., Cryptotermes sp.), and Rhinotermitidae (e.g., Reticulitermes sp., Coptotermes sp., Heterotermes tenuis Hagen) families, the eastern subterranean termite (Reticulitermes flavipes Kollar), western subterranean termite (Reticulitermes hesperus Banks), Formosan subterranean termite (Coptotermes formosanus Shiraki), West Indian drywood termite (Incisitermes immigrans Snyder), powder post termite (Cryptotermes brevis Walker), drywood termite (Incisitermes snyderi Light), southeastern subterranean termite (Reticulitermes virginicus Banks), western drywood termite (Incisitermes minor Hagen), arboreal termites such as Nasutitermes sp. and other termites of economic importance; insect pests of the order Thysanura such as silverfish (Lepisma saccharina Linnaeus) and firebrat (Thermobia domestica Packard); insect pests of the order Mallophaga and including the head louse (Pediculus humanus capitis De Geer), body louse (Pediculus humanus Linnaeus), chicken body louse (Menacanthus stramineus Nitszch), dog biting louse (Trichodectes canis De Geer), fluff louse (Goniocotes gallinae De Geer), sheep body louse (Bovicola ovis Schrank), short-nosed cattle louse (Haematopinus eurysternus Nitzsch), long-nosed cattle louse (Linognathus vituli Linnaeus) and other sucking and chewing parasitic lice that attack man and animals; insect pests of the order Siphonoptera including the oriental rat flea (Xenopsylla cheopis Rothschild), cat flea (Ctenocephalides felis Bouche), dog flea (Ctenocephalides canis Curtis), hen flea (Ceratophyllus gallinae Schrank), sticktight flea (Echidnophaga gallinacea Westwood), human flea (Pulex irritans Linnaeus) and other fleas afflicting mammals and birds. Additional arthropod pests covered include: spiders in the order Araneae such as the brown recluse spider (Loxosceles reclusa Gertsch & Mulaik) and the black widow spider (Latrodectus mactans Fabricius), and centipedes in the order Scutigeromorpha such as the house centipede (Scutigera coleoptrata Linnaeus). Compounds of the present invention also have activity on members of the Classes Nematoda, Cestoda, Trematoda, and Acanthocephala including economically important members of the orders Strongylida, Ascaridida, Oxyurida, Rhabditida, Spirurida, and Enoplida such as but not limited to economically important agricultural pests (i.e. root knot nematodes in the genus Meloidogyne, lesion nematodes in the genus Pratylenchus, stubby root nematodes in the genus Trichodorus, etc.) and animal and human health pests (i.e. all economically important flukes, tapeworms, and roundworms, such as Strongylus vulgaris in horses, Toxocara canis in dogs, Haemonchus contortus in sheep, Dirofilaria immitis Leidy in dogs, Anoplocephala perfoliata in horses, Fasciola hepatica Linnaeus in ruminants, etc.).

Compounds of the invention show particularly high activity against pests in the order Lepidoptera (e.g., Alabama argillacea Hübner (cotton leaf worm), Archips argyrospila Walker (fruit tree leaf roller), A. rosana Linnaeus (European leaf roller) and other Archips species, Chilo suppressalis Walker (rice stem borer), Cnaphalocrosis medinalis Guenee (rice leaf roller), Crambus caliginosellus Clemens (corn root webwomm), Crambus teterrellus Zincken (bluegrass webworm), Cydia pomonella Linnaeus (codling moth), Earias insulana Boisduval (spiny bollworm), Earias vittella Fabricius (spotted bollworm), Helicoverpa armigera Hübner (American bollworm), Helicoverpa zea Boddie (corn earworm), Heliothis virescens Fabricius (tobacco budworm), Herpetogramma licarsisalis Walker (sod webworm), Lobesia botrana Denis & Schiffermüller (grape berry moth), Pectinophora gossypiella Saunders (pink bollworm), Phyllocnistis citrella Stainton (citrus leafininer), Pieris brassicae Linnaeus (large white butterfly), Pieris rapae Linnaeus (small white butterfly), Plutella xylostella Linnaeus (diamondback moth), Spodoptera exigua Hübner (beet armyworm), Spodoptera litura Fabricius (tobacco cutworm, cluster caterpillar), Spodoptera frugiperda J. E. Smith (fall armyworm), Trichoplusia ni Hübner (cabbage looper) and Tuta absoluta Meyrick (tomato leafminer)).

Compounds of the invention also have significant activity on members from the order Homopteraincluding: Acyrthosiphon pisum Harris (pea aphid), Aphis craccivora Koch (cowpea aphid), Aphis fabae Scopoli (black bean aphid), Aphis gossypii Glover (cotton aphid, melon aphid), Aphis pomi De Geer (apple aphid), Aphis spiraecola Patch (spirea aphid), Aulacorthum solani Kaltenbach (foxglove aphid), Chaetosiphon fragaefolii Cockerell (strawberry aphid), Diuraphis noxia Kurdjumov/Mordvilko (Russian wheat aphid), Dysaphis plantaginea Paaserini (rosy apple aphid), Eriosoma lanigerum Hausmann (woolly apple aphid), Hyalopterus pruni Geoffroy (mealy plum aphid), Lipaphis erysimi Kaltenbach (turnip aphid), Metopolophium dirrhodum Walker (cereal aphid), Macrosiphum euphorbiae Thomas (potato aphid), Myzus persicae Sulzer (peach-potato aphid, green peach aphid), Nasonovia ribisnigri Mosley (lettuce aphid), Pemphigus spp. (root aphids and gall aphids), Rhopalosiphum maidis Fitch (corn leaf aphid), Rhopalosiphum padi Linnaeus (bird cherry-oat aphid), Schizaphis graminum Rondani (greenbug), Sitobion avenae Fabricius (English grain aphid), Therioaphis maculata Buckton (spotted alfalfa aphid), Toxoptera aurantii Boyer de Fonscolombe (black citrus aphid), and Toxoptera citricida Kirkaldy (brown citrus aphid); Adelges spp. (adelgids); Phylloxera devastatrix Pergande (pecan phylloxera); Bemisia tabaci Gennadius (tobacco whitefly, sweetpotato whitefly), Bemisia argentifolii Bellows & Perring (silverleaf whitefly), Dialeurodes citri Ashmead (citrus whitefly) and Trialeurodes vaporariorum Westwood (greenhouse whitefly); Empoasca fabae Harris (potato leafhopper), Laodelphax striatellus Fallen (smaller brown planthopper), Macrolestes quadrilineatus Forbes (aster leafhopper), Nephotettix cinticeps Uhler (green leafhopper), Nephotettix nigropictus Stål (rice leafhopper), Nilaparvata lugens Stal (brown planthopper), Peregrinus maidis Ashmead (corn planthopper), Sogatella furcifera Horvath (white-backed planthopper), Sogatodes orizicola Muir (rice delphacid), Typhlocyba pomaria McAtee white apple leafhopper, Erythroneoura spp. (grape leafhoppers); Magicidada septendecim Linnaeus (periodical cicada); Icerya purchasi Maskell (cottony cushion scale), Quadraspidiotus perniciosus Comstock (San Jose scale); Planococcus citri Risso (citrus mealybug); Pseudococcus spp. (other mealybug complex); Cacopsylla pyricola Foerster (pear psylla), Trioza diospyri Ashmead (persirmmon psylla).

Compounds of this invention also have activity on members from the order Hemiptera including: Acrosternum hilare Say (green stink bug), Anasa tristis De Geer (squash bug), Blissus leucopterus leucopterus Say (chinch bug), Cimex lectularius Linnaeus (bed bug) Corythuca gossypii Fabricius (cotton lace bug), Cyrtopeltis modesta Distant (tomato bug), Dysdercus suturellus Herrich-Schäffer (cotton stainer), Euchistus servus Say (brown stink bug), Euchistus variolarius Palisot de Beauvois (one-spotted stink bug), Graptosthetus spp. (complex of seed bugs), Leptoglossus corculus Say (leaf-footed pine seed bug), Lygus lineolaris Palisot de Beauvois (tarnished plant bug), Nezara viridula Linnaeus (southern green stink bug), Oebalus pugnax Fabricius (rice stink bug), Oncopeltus fasciatus Dallas (large milkweed bug), Pseudatomoscelis seriatus Reuter (cotton fleahopper). Other insect orders controlled by compounds of the invention include Thysanoptera (e.g., Frankliniella occidentalis Pergande (western flower thrips), Scirthothrips citri Moulton (citrus thrips), Sericothrips variabilis Beach (soybean thrips), and Thrips tabaci Lindeman (onion thrips); and the order Coleoptera (e.g., Leptinotarsa decemlineata Say (Colorado potato beetle), Epilachna varivestis Mulsant (Mexican bean beetle) and wireworms of the genera Agriotes, Athous or Limonius).

Note that some contemporary classification systems place Homopteraas a suborder within the order Hemiptera.

Of note is use of compounds of this invention for controlling western flower thrips (Frankliniella occidentalis). Of note is use of compounds of this invention for controlling potato leafhopper (Empoasca fabae). Of note is use of compounds of this invention for controlling diamondback moth (Plutella xylostella). Of note is use of compounds of this invention for controlling fall armyworm (Spodoptera fugiperda).

Compounds of this invention can also be mixed with one or more other biologically active compounds or agents including insecticides, fungicides, nematocides, bactericides, acaricides, herbicides, growth regulators such as rooting stimulants, chemosterilants, semiochemicals, repellents, attractants, pheromones, feeding stimulants, other biologically active compounds or entomopathogenic bacteria, virus or fungi to form a multi-component pesticide giving an even broader spectrum of agronomic and nonagronomic utility. Thus the present invention also pertains to a composition comprising a biologically effective amount of a compound of Formula 1, an N-oxide or a salt thereof, and an effective amount of at least one additional biologically active compound or agent and can further comprise at least one of a surfactant, a solid diluent or a liquid diluent. The other biologically active compounds or agents can be formulated in compositions comprising at least one of a surfactant, solid or liquid diluent. For mixtures of the present invention, the other biologically active compounds or agents can be formulated together with the present compounds, including the compounds of Formula 1, to form a premix, or the other biologically active compounds or agents can be formulated separately from the present compounds, including the compounds of Formula 1, and the two formulations combined together before application (e.g., in a spray tank) or, alternatively, applied in succession.

Other biologically active compounds or agents useful in the compositions of the present invention can be selected from invertebrate pest control agents having a different mode of action or a different chemical class including macrocyclic lactones, neonicotinoids, octopamine receptor ligands, ryanodine receptor ligands, ecdysone agonists, sodium channel modulators, chitin synthesis inhibitors, nereisotoxin analogs, mitochondrial electron transport inhibitors, cholinesterase inhibitors, cyclodiene insecticides, molting inhibitors, GABA (γ-aminobutyric acid)-regulated chloride channel blockers, juvenile hormone mimics, lipid biosynthesis inhibitors and biological agents including nucleopolyhedro virus (NPV), a member of Bacillus thuringiensis, an encapsulated delta-endotoxin of Bacillus thuringiensis; and a naturally occurring or a genetically modified viral insecticide. Of note are additional biologically active compounds or agents selected from insecticides of the group consisting of pyrethroids, carbamates, neonicotinoids, neuronal sodium channel blockers, insecticidal macrocyclic lactones, γ-aminobutyric acid (GABA) antagonists, insecticidal ureas and juvenile hormone mimics, a member of Bacillus thuringiensis, a Bacillus thuringiensis delta-endotoxin, and a naturally occurring or a genetically modified viral insecticide.

Of note is a composition of the present invention wherein at least one additional biologically active compound or agent is selected from insecticides of the group consisting of macrocyclic lactones, neonicotinoids, octopamine receptor ligands, ryanodine receptor ligands, ecdysone agonists, sodium channel modulators, chitin synthesis inhibitors, nereisotoxin analogs, mitochondrial electron transport inhibitors, cholinesterase inhibitors, cyclodiene insecticides, molting inhibitors, GABA-regulated chloride channel blockers, juvenile hormone mimics, biological agents, and lipid biosynthesis inhibitors.

Also of note is a composition of the present invention wherein at least one additional biologically active compound or agent is selected from insecticides of the group consisting of pyrethroids, carbamates, neonicotinoids, neuronal sodium channel blockers, insecticidal macrocyclic lactones, γ-aminobutyric acid (GABA) antagonists, insecticidal ureas and juvenile hormone mimics, a member of Bacillus thuringiensis, a Bacillus thuringiensis delta-endotoxin, and a naturally occurring or a genetically modified viral insecticide.

Examples of such biologically active compounds or agents with which compounds of this invention can be formulated are: insecticides such as abamectin, acephate, acetamiprid, acetoprole, amidoflumet (S-1955), avermectin, azadirachtin, azinphos-methyl, bifenthrin, bifenazate, bistrifluoron, buprofezin, carbofuran, cartap, chlorfenapyr, chlorfluazuron, chlorantraniliprole (DPX-E2Y45), chlorpyrifos, chlorpyrifos-methyl, chromafenozide, clothianidin, cyflumetofen, cyfluthrin, beta-cyfluthrin, cyhalothrin, gamma-cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, monocrotophos, methoxyfenozide, monocrotophos, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, protrifenbute, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tolfenpyrad, tralomethrin, triazamate, trichlorfon and triflumuron; fungicides such as acibenzolar, aldimorph, amisulbrom, azaconazole, azoxystrobin, benalaxyl, benomyl, benthiavalicarb, benthiavalicarb-isopropyl, binomial, biphenyl, bitertanol, blasticidin-S, Bordeaux mixture (Tribasic copper sulfate), boscalid/nicobifen, bromuconazole, bupirimate, buthiobate, carboxin, carpropamid, captafol, captan, carbendazim, chloroneb, chlorothalonil, chlozolinate, clotrimazole, copper oxychloride, copper salts such as copper sulfate and copper hydroxide, cyazofamid, cyflunamid, cymoxanil, cyproconazole, cyprodinil, dichlofluanid, diclocymet, diclomezine, dicloran, diethofencarb, difenoconazole, dimethomorph, dimoxystrobin, diniconazole, diniconazole-M, dinocap, discostrobin, dithianon, dodemorph, dodine, econazole, etaconazole, edifenphos, epoxiconazole, ethaboxam, ethirimol, ethridiazole, famoxadone, fenamidone, fenarimol, fenbuconazole, fencaramid, fenfuram, fenhexamide, fenoxanil, fenpiclonil, fenpropidin, fenpropimorph, fentin acetate, fentin hydroxide, ferbam, ferfurazoate, ferimzone, fluazinam, fludioxonil, flumetover, fluopicolide, fluoxastrobin, fluquinconazole, fluquinconazole, flusilazole, flusulfamide, flutolanil, flutriafol, folpet, fosetyl-aluminum, fuberidazole, furalaxyl, furametapyr, hexaconazole, hymexazole, guazatine, imazalil, imibenconazole, iminoctadine, iodicarb, ipconazole, iprobenfos, iprodione, iprovalicarb, isoconazole, isoprothiolane, kasugamycin, kresoxim-methyl, mancozeb, mandipropamid, maneb, mapanipyrin, mefenoxam, mepronil, metalaxyl, metconazole, methasulfocarb, metiram, metominostrobin/fenominostrobin, mepanipyrim, metrafenone, miconazole, myclobutanil, neo-asozin (ferric methanearsonate), nuarimol, octhilinone, ofurace, orysastrobin, oxadixyl, oxolinic acid, oxpoconazole, oxycarboxin, paclobutrazol, penconazole, pencycuron, penthiopyrad, perfurazoate, phosphonic acid, phthalide, picobenzamid, picoxystrobin, polyoxin, probenazole, prochloraz, procymidone, proparnocarb, propamocarb-hydrochloride, propiconazole, propineb, proquinazid, prothioconazole, pyraclostrobin, pryazophos, pyrifenox, pyrimethanil, pyrifenox, pyroInitrine, pyroquilon, quinconazole, quinoxyfen, quintozene, silthiofam, simeconazole, spiroxamine, streptomycin, sulfur, tebuconazole, techrazene, tecloftalam, tecnazene, tetraconazole, thiabendazole, thifluzamide, thiophanate, thiophanate-methyl, thiram, tiadinil, tolclofos-methyl, tolyfluanid, triadimefon, triadimenol, triarimol, triazoxide, tridemorph, trimoprhamide tricyclazole, trifloxystrobin, triforine, triticonazole, uniconazole, validamycin, vinclozolin, zineb, ziram, and zoxamide; nematocides such as aldicarb, imicyafos, oxamyl and fenamiphos; bactericides such as streptomycin; acaricides such as amitraz, chinomethionat, chlorobenzilate, cyhexatin, dicofol, dienochlor, etoxazole, fenazaquin, fenbutatin oxide, fenpropathrin, fenpyroximate, hexythiazox, propargite, pyridaben and tebufenpyrad; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied invertebrate pest control compounds of this invention may be synergistic with the expressed toxin proteins.

General references for these agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2^{nd} Edition, L. G. Copping, Ed., British Crop Protection Council, Farnham, Surrey, U.K., 2001.

Of note is a composition of the present invention wherein at least one additional biologically active compound or agent is selected from the group consisting of abamectin, acephate, acetamiprid, acetoprole, aldicarb, amidoflumet, amitraz, avermectin, azadirachtin, azinphos-methyl, bifenthrin, bifenazate, bistrifluoron, buprofezin, carbofuran, cartap, chinomethionat, chlorfenapyr, chlorfluazuron, chlorantraniliprole, chlorpyrifos, chlorpyrifos-methyl, chlorobenzilate, chromafenozide, clothianidin, cyflumetofen, cyfluthrin, beta-cyfluthrin, cyhalothrin, gamma-cyhalothrin, lambda-cyhalothrin, cyhexatin, cypermethrin, cyromazine, deltamethrin, diafenthiuron, diazinon, dicofol, dieldrin, dienochlor, diflubenzuron, dimefluthrin, dimethoate, dinotefuran, diofenolan, emamectin, endosulfan, esfenvalerate, ethiprole, etoxazole, fenamiphos, fenazaquin, fenbutatin oxide, fenothiocarb, fenoxycarb, fenpropathrin, fenpyroximate, fenvalerate, fipronil, flonicamid, flubendiamide, flucythrinate, tau-fluvalinate, flufenerim, flufenoxuron, fonophos, halofenozide, hexaflumuron, hexythiazox, hydramethylnon, imicyafos, imidacloprid, indoxacarb, isofenphos, lufenuron, malathion, metaflumizone, metaldehyde, methamidophos, methidathion, methomyl, methoprene, methoxychlor, methoxyfenozide, metofluthrin, monocrotophos, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, spinetoram, spinosad, spiridiclofen, spiromesifen, spirotetramat, sulprofos, tebufenozide, tebufenpyrad, teflubenzuron, tefluthrin, terbufos, tetrachlorvinphos, thiacloprid, thiamethoxam, thiodicarb, thiosultap-sodium, tolfenpyrad, tralomethrin, triazamate, trichlorfon, triflumuron, Bacillus thuringiensis subsp. aizawai, Bacillus thuringiensis subsp. kurstaki, nucleopolyhedro virus, an encapsulated delta-endotoxin of Bacillus thuringiensis, baculovirus, entomopathogenic bacteria, entomopathogenic virus and entomopathogenic fungi.

Of particular note is a composition of the present invention wherein the at least one additional biologically active compound or agent is selected from the group consisting of abamectin, acetamiprid, amitraz, avermectin, azadirachtin, bifenthrin, buprofezin, cartap, chlorantraniliprole, chlorfenapyr, chlorpyrifos, clothianidin, cyfluthrin, beta-cyfluthrin, cyhalothrin, lambda-cyhalothrin, cypermethrin, cyromazine, deltamethrin, dieldrin, dinotefuran, diofenolan, emamectin, endosulfan, esfenvalerate, ethiprole, fenothiocarb, fenoxycarb, fenvalerate, fipronil, flonicamid, flubendiamide, flufenoxuron, hexaflumuron, hydramethylnon, imidacloprid, indoxacarb, lufenuron, metaflumizone, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, tebufenozide, thiacloprid, thiamethoxam, thiodicarb, thiosultap-sodium, tralomethrin, triazamate, triflumuron, Bacillus thuringiensis subsp. aizawai, Bacillus thuringiensis subsp. kurstaki, nucleopolyhedro virus and an encapsulated delta-endotoxin of Bacillus thuringiensis.

Also of note is a composition of the present invention wherein the at least one additional biologically active compound or agent is selected from the group consisting of abamectin, acephate, acetamiprid, acetoprole, amidoflumet (S-1955), avermectin, azadirachtin, azinphos-methyl, bifenthrin, bifenazate, bistrifluoron, buprofezin, carbofuran, cartap, chlorfenapyr, chlorfluazuron, chlorpyrifos, chlorpyrifos-methyl, chromafenozide, clothianidin, cyfluthrin, beta-cyfluthrin, cyhalothrin, lambda-cyhalothrin, cypermethrin, cyromazine, deltamethrin, diafenthiuron, diazinon, dieldrin, diflubenzuron, dimethoate, dinotefuran, diofenolan, emamectin, endosulfan, esfenvalerate, ethiprole, fenothicarb, fenoxycarb, fenpropathrin, fenvalerate, fipronil, flonicamid, flubendiamide, flucythrinate, tau-fluvalinate, flufenerim (UR-50701), flufenoxuron, gamma-chalothrin, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, methoxyfenozide, metofluthrin, monocrotophos, methoxyfenozide, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, protrifenbute, pymetrozine, pyrethrin, pyridalyl, pyriproxyfen, rotenone, ryanodine, S1812 (Valent), spinosad, spiridiclofen, spiromesifen (BSN 2060), sulprofos, tebufenozide, teflubenzuron, tefluthrin, terbufos, tetrachlorvinphos, thiacloprid, thiamethoxam, thiodicarb, thiosultap-sodium, tolfenpyrad, tralomethrin, triazamate, trichlorfon, triflumuron, aldicarb, fenamiphos, amitraz, chinomethionat, chlorobenzilate, cyhexatin, dicofol, dienochlor, etoxazole, fenazaquin, fenbutatin oxide, fenpyroximate, hexythiazox, propargite, pyridaben, tebufenpyrad, Bacillus thuringiensis aizawai, Bacillus thuringiensis kurstaki, Bacillus thuringiensis delta endotoxin, baculovirus, entomopathogenic bacteria, entomopathogenic virus and entomopathogenic fungi.

Of further note is a composition of the present invention wherein the at least one additional biologically active compound or agent is selected from the group consisting of cypermethrin, cyhalothrin, cyfluthrin- and beta-cyfluthrin, esfenvalerate, fenvalerate, tralomethrin, fenothicarb, methomyl, oxamyl, thiodicarb, acetamiprid, clothianidin, imidacloprid, thiamethoxam, thiacloprid, indoxacarb, spinosad, abamectin, avermectin, emamectin, endosulfan, ethiprole, fipronil, flufenoxuron, triflumuron, diofenolan, pyriproxyfen, pymetrozine, amitraz, Bacillus thuringiensis aizawai, Bacillus thuringiensis kurstaki, Bacillus thuringiensis delta endotoxin and entomophagous fungi.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of invertebrate pests controlled beyond the spectrum controlled by the compound of Formula 1 alone.

In certain instances, combinations of a compound of this invention with other biologically active (particularly invertebrate pest control) compounds or agents (i.e. active ingredients) can result in a greater-than-additive (i.e. synergistic) effect. Reducing the quantity of active ingredients released in the environment while ensuring effective pest control is always desirable. When synergism of invertebrate pest control active ingredients occurs at application rates giving agronomically satisfactory levels of invertebrate pest control, such combinations can be advantageous for reducing crop production cost and decreasing environmental load.

Of note is a combination of a compound of Formula 1 with at least one other invertebrate pest control active ingredient. Of particular note is such a combination where the other invertebrate pest control active ingredient has different site of action from the compound of Formula 1. In certain instances, a combination with at least one other invertebrate pest control active ingredient having a similar spectrum of control but a different site of action will be particularly advantageous for resistance management. Thus, a composition of the present invention can further comprise a biologically effective amount of at least one additional invertebrate pest control active ingredient having a similar spectrum of control but a different site of action. Contacting a plant genetically modified to express an invertebrate pest compound (e.g., protein) or the locus of the plant with a biologically effective amount of a compound of this invention can also provide a broader spectrum of plant protection and be advantageous for resistance management.

Table A lists specific combinations of a compound of Formula 1 with other invertebrate pest control agents illustrative of the mixtures, compositions and methods of the present invention. The first column of Table A lists the specific invertebrate pest control agents (e.g., “Abamectin” in the first line). The second column of Table A lists the mode of action (if known) or chemical class of the invertebrate pest control agents. The third column of Table A lists embodiment(s) of ranges of weight ratios for rates at which the invertebrate pest control agent can be applied relative to a compound of Formula 1, an N-oxide, or a salt thereof, (e.g., “50:1 to 1:50” of abamectin relative to a compound of Formula 1 by weight). Thus, for example, the first line of Table A specifically discloses the combination of a compound of Formula 1 with abamectin can be applied in a weight ratio between 50:1 to 1:50. The remaining lines of Table A are to be construed similarly. Of further note Table A lists specific combinations of a compound of Formula 1 with other invertebrate pest control agents illustrative of the mixtures, compositions and methods of the present invention and includes additional embodiments of weight ratio ranges for application rates.

TABLE A

| Invertebrate Pest | Mode of Action or | Typical |
|---|---|---|
| Control Agent | Chemical Class | Weight Ratio |
| Abamectin | macrocyclic lactones | 50:1 to 1:50 |
| Acetamiprid | neonicotinoids | 150:1 to 1:200 |
| Amitraz | octopamine receptor ligands | 200:1 to 1:100 |
| Avermectin | macrocyclic lactones | 50:1 to 1:50 |
| Azadirachtin | ecdysone agonists | 100:1 to 1:120 |
| Beta-cyfluthrin | sodium channel modulators | 150:1 to 1:200 |
| Bifenthrin | sodium channel modulators | 100:1 to 1:10 |
| Buprofezin | chitin synthesis inhibitors | 500:1 to 1:50 |
| Cartap | nereistoxin analogs | 100:1 to 1:200 |
| Chlorantraniliprole | ryanodine receptor ligands | 100:1 to 1:120 |
| Chlorfenapyr | mitochondrial electron | 300:1 to 1:200 |
| | transport inhibitors | |
| Chlorpyrifos | cholinesterase inhibitors | 500:1 to 1:200 |
| Clothianidin | neonicotinoids | 100:1 to 1:400 |
| Cyfluthrin | sodium channel modulators | 150:1 to 1:200 |
| Cyhalothrin | sodium channel modulators | 150:1 to 1:200 |
| Cypermethrin | sodium channel modulators | 150:1 to 1:200 |
| Cyromazine | chitin synthesis inhibitors | 400:1 to 1:50 |
| Deltamethrin | sodium channel modulators | 50:1 to 1:400 |
| Dieldrin | cyclodiene insecticides | 200:1 to 1:100 |
| Dinotefuran | neonicotinoids | 150:1 to 1:200 |
| Diofenolan | molting inhibitor | 150:1 to 1:200 |
| Emamectin | macrocyclic lactones | 50:1 to 1:10 |
| Endosulfan | cyclodiene insecticides | 200:1 to 1:100 |
| Esfenvalerate | sodium channel modulators | 100:1 to 1:400 |
| Ethiprole | GABA-regulated chloride | 200:1 to 1:100 |
| | channel blockers | |
| Fenothiocarb | | 150:1 to 1:200 |
| Fenoxycarb | juvenile hormone mimics | 500:1 to 1:100 |
| Fenvalerate | sodium channel modulators | 150:1 to 1:200 |
| Fipronil | GABA-regulated chloride | 150:1 to 1:100 |
| | channel blockers | |
| Flonicamid | | 200:1 to 1:100 |
| Flubendiamide | ryanodine receptor ligands | 100:1 to 1:120 |
| Flufenoxuron | chitin synthesis inhibitors | 200:1 to 1:100 |
| Hexaflumuron | chitin synthesis inhibitors | 300:1 to 1:50 |
| Hydramethylnon | mitochondrial electron | 150:1 to 1:250 |
| | transport inhibitors | |
| Imidacloprid | neonicotinoids | 1000:1 to 1:1000 |
| Indoxacarb | sodium channel modulators | 200:1 to 1:50 |
| Lambda-cyhalothrin | sodium channel modulators | 50:1 to 1:250 |
| Lufenuron | chitin synthesis inhibitors | 500:1 to 1:250 |
| Metaflumizone | | 200:1 to 1:200 |
| Methomyl | cholinesterase inhibitors | 500:1 to 1:100 |
| Methoprene | juvenile hormone mimics | 500:1 to 1:100 |
| Methoxyfenozide | ecdysone agonists | 50:1 to 1:50 |
| Nitenpyram | neonicotinoids | 150:1 to 1:200 |
| Nithiazine | neonicotinoids | 150:1 to 1:200 |
| Novaluron | chitin synthesis inhibitors | 500:1 to 1:150 |
| Oxamyl | cholinesterase inhibitors | 200:1 to 1:200 |
| Pymetrozine | | 200:1 to 1:100 |
| Pyrethrin | sodium channel modulators | 100:1 to 1:10 |
| Pyridaben | mitochondrial electron | 200:1 to 1:100 |
| | transport inhibitors | |
| Pyridalyl | | 200:1 to 1:100 |
| Pyriproxyfen | juvenile hormone mimics | 500:1 to 1:100 |
| Ryanodine | ryanodine receptor ligands | 100:1 to 1:120 |
| Spinetoram | macrocyclic lactones | 150:1 to 1:100 |
| Spinosad | macrocyclic lactones | 500:1 to 1:10 |
| Spirodiclofen | lipid biosynthesis inhibitors | 200:1 to 1:200 |
| Spiromesifen | lipid biosynthesis inhibitors | 200:1 to 1:200 |
| Tebufenozide | ecdysone agonists | 500:1 to 1:250 |
| Thiacloprid | neonicotinoids | 100:1 to 1:200 |
| Thiamethoxam | neonicotinoids | 1250:1 to 1:1000 |
| Thiodicarb | cholinesterase inhibitors | 500:1 to 1:400 |
| Thiosultap-sodium | | 150:1 to 1:100 |
| Tralomethrin | sodium channel modulators | 150:1 to 1:200 |
| Triazamate | cholinesterase inhibitors | 250:1 to 1:100 |
| Triflumuron | chitin synthesis inhibitors | 200:1 to 1:100 |
| Bacillus thuringiensis | biological agents | 50:1 to 1:10 |
| Bacillus thuringiensis | biological agents | 50:1 to 1:10 |
| delta-endotoxin | | |
| NPV (e.g., Gemstar) | biological agents | 50:1 to 1:10 |

One embodiment of invertebrate pest control agents (e.g., insecticides and acaricides) for mixing with compounds of this invention include sodium channel modulators such as bifenthrin, cypermethrin, cyhalothrin, lambda-cyhalothrin, cyfluthrin, beta-cyfluthrin, deltamethrin, dimefluthrin, esfenvalerate, fenvalerate, indoxacarb, metofluthrin, profluthrin, pyrethrin and tralomethrin; cholinesterase inhibitors such as chlorpyrifos, methomyl, oxamyl, thiodicarb and triazamate; neonicotinoids such as acetamiprid, clothianidin, dinotefuran, imidacloprid, nitenpyram, nithiazine, thiacloprid and thiamethoxam; insecticidal macrocyclic lactones such as spinetoram, spinosad, abamectin, avermectin and emamectin; GABA (γ-aminobutyric acid)-regulated chloride channel blockers such as endosulfan, ethiprole and fipronil; chitin synthesis inhibitors such as buprofezin, cyromazine, flufenoxuron, hexaflumuron, lufenuron, novaluron, noviflumuron and triflumuron; juvenile hormone mimics such as diofenolan, fenoxycarb, methoprene and pyriproxyfen; octopamine receptor ligands such as amitraz; ecdysone agonists such as azadirachtin, methoxyfenozide and tebufenozide; ryanodine receptor ligands such as ryanodine, anthranilic diamides such as chlorantraniliprole (see U.S. Pat. No. 6,747,047, PCT Publications WO 2003/015518 and WO 2004/067528) and flubendiamide (see U.S. Pat. No. 6,603,044); nereistoxin analogs such as cartap; mitochondrial electron transport inhibitors such as chlorfenapyr, hydramethylnon and pyridaben; lipid biosynthesis inhibitors such as spirodiclofen and spiromesifen; cyclodiene insecticides such as dieldrin; cyflumetofen; fenothiocarb; flonicamid; metaflumizone; pyrafluprole; pyridalyl; pyriprole; pymetrozine; spirotetramat; and thiosultap-sodium. One embodiment of biological agents for mixing with compounds of this invention include nucleopolyhedro virus such as HzNPV and AfNPV; Bacillus thuringiensis and encapsulated delta-endotoxins of Bacillus thuringiensis such as Cellcap, MPV and MPVII; as well as naturally occurring and genetically modified viral insecticides including members of the family Baculoviridae as well as entomophagous fungi. Of note is the composition of the present invention wherein the at least one additional biologically active compound or agent is selected from the Invertebrate Pest Control Agents listed in Table A above. Also of note is the composition of the present invention wherein the at least one additional biologically active compound or agent is selected from the group consisting of cypermethrin, cyhalothrin, cyfluthrin, beta-cyfluthrin, esfenvalerate, fenvalerate, tralomethrin, fenothiocarb, methomyl, oxamyl, thiodicarb, acetamiprid, clothianidin, imidacloprid, thiamethoxam, thiacloprid, indoxacarb, spinosad, abamectin, avermectin, emamectin, endosulfan, ethiprole, fipronil, flufenoxuron, triflumuron, diofenolan, pyriproxyfen, pymetrozine, amitraz, Bacillus thuringiensis aisawai, Bacillus thuringiensis kurstaki, Bacillus thuringiensis delta endotoxin and entomophagous fungi.

The weight ratios of a compound, including a compound of Formula 1, an N-oxide or a salt thereof, to the additional invertebrate pest control agent typically are between 1000:1 and 1:1000, with one embodiment being between 500:1 and 1:500, another embodiment being between 250:1 and 1:200 and another embodiment being between 100:1 and 1:50.

Listed below in Table B are embodiments of specific compositions comprising a compound of Formula 1 (compound numbers refer to compounds in Index Tables A-B) and an additional invertebrate pest control agent.

TABLE B

| | | | Invertebrate Pest |
|---|---|---|---|
| Mixture No. | Comp. No. | and | Control Agent |
| A-1 | 7 | and | Abamectin |
| A-2 | 7 | and | Acetamiprid |
| A-3 | 7 | and | Amitraz |
| A-4 | 7 | and | Avermectin |
| A-5 | 7 | and | Azadirachtin |
| A-6 | 7 | and | Beta-cyfluthrin |
| A-7 | 7 | and | Bifenthrin |
| A-8 | 7 | and | Buprofezin |
| A-9 | 7 | and | Cartap |
| A-10 | 7 | and | Chlorantraniliprole |
| A-11 | 7 | and | Chlorfenapyr |
| A-12 | 7 | and | Chlorpyrifos |
| A-13 | 7 | and | Clothianidin |
| A-14 | 7 | and | Cyfluthrin |
| A-15 | 7 | and | Cyhalothrin |
| A-16 | 7 | and | Cypermethrin |
| A-17 | 7 | and | Cyromazine |
| A-18 | 7 | and | Deltamethrin |
| A-19 | 7 | and | Dieldrin |
| A-20 | 7 | and | Dinotefuran |
| A-21 | 7 | and | Diofenolan |
| A-22 | 7 | and | Emamectin |
| A-23 | 7 | and | Endosulfan |
| A-24 | 7 | and | Esfenvalerate |
| A-25 | 7 | and | Ethiprole |
| A-26 | 7 | and | Fenothiocarb |
| A-27 | 7 | and | Fenoxycarb |
| A-28 | 7 | and | Fenvalerate |
| A-29 | 7 | and | Fipronil |
| A-30 | 7 | and | Flonicamid |
| A-31 | 7 | and | Flubendiamide |
| A-32 | 7 | and | Flufenoxuron |
| A-33 | 7 | and | Hexaflumuron |
| A-34 | 7 | and | Hydramethylnon |
| A-35 | 7 | and | Imidacloprid |
| A-36 | 7 | and | Indoxacarb |
| A-37 | 7 | and | Lambda-cyhalothrin |
| A-38 | 7 | and | Lufenuron |
| A-39 | 7 | and | Metaflumizone |
| A-40 | 7 | and | Methomyl |
| A-41 | 7 | and | Methoprene |
| A-42 | 7 | and | Methoxyfenozide |
| A-43 | 7 | and | Nitenpyram |
| A-44 | 7 | and | Nithiazine |
| A-45 | 7 | and | Novaluron |
| A-46 | 7 | and | Oxamyl |
| A-47 | 7 | and | Pymetrozine |
| A-48 | 7 | and | Pyrethrin |
| A-49 | 7 | and | Pyridaben |
| A-50 | 7 | and | Pyridalyl |
| A-51 | 7 | and | Pyriproxyfen |
| A-52 | 7 | and | Ryanodine |
| A-53 | 7 | and | Spinetoram |
| A-54 | 7 | and | Spinosad |
| A-55 | 7 | and | Spirodiclofen |
| A-56 | 7 | and | Spiromesifen |
| A-57 | 7 | and | Tebufenozide |
| A-58 | 7 | and | Thiacloprid |
| A-59 | 7 | and | Thiamethoxam |
| A-60 | 7 | and | Thiodicarb |
| A-61 | 7 | and | Thiosultap-sodium |
| A-62 | 7 | and | Tralomethrin |
| A-63 | 7 | and | Triazamate |
| A-64 | 7 | and | Triflumuron |
| A-65 | 7 | and | Bacillus thuringiensis |
| A-66 | 7 | and | Bacillus thuringiensis |
| | | | delta-endotoxin |
| A-67 | 7 | and | NPV (e.g., Gemstar) |
| B-1 | 10 | and | Abamectin |
| B-2 | 10 | and | Acetamiprid |
| B-3 | 10 | and | Amitraz |
| B-4 | 10 | and | Avermectin |
| B-5 | 10 | and | Azadirachtin |
| B-6 | 10 | and | Beta-cyfluthrin |
| B-7 | 10 | and | Bifenthrin |
| B-8 | 10 | and | Buprofezin |
| B-9 | 10 | and | Cartap |
| B-10 | 10 | and | Chlorantraniliprole |
| B-11 | 10 | and | Chlorfenapyr |
| B-12 | 10 | and | Chlorpyrifos |
| B-13 | 10 | and | Clothianidin |
| B-14 | 10 | and | Cyfluthrin |
| B-15 | 10 | and | Cyhalothrin |
| B-16 | 10 | and | Cypermethrin |
| B-17 | 10 | and | Cyromazine |
| B-18 | 10 | and | Deltamethrin |
| B-19 | 10 | and | Dieldrin |
| B-20 | 10 | and | Dinotefuran |
| B-21 | 10 | and | Diofenolan |
| B-22 | 10 | and | Emamectin |
| B-23 | 10 | and | Endosulfan |
| B-24 | 10 | and | Esfenvalerate |
| B-25 | 10 | and | Ethiprole |
| B-26 | 10 | and | Fenothiocarb |
| B-27 | 10 | and | Fenoxycarb |
| B-28 | 10 | and | Fenvalerate |
| B-29 | 10 | and | Fipronil |
| B-30 | 10 | and | Flonicamid |
| B-31 | 10 | and | Flubendiamide |
| B-32 | 10 | and | Flufenoxuron |
| B-33 | 10 | and | Hexaflumuron |
| B-34 | 10 | and | Hydramethylnon |
| B-35 | 10 | and | Imidacloprid |
| B-36 | 10 | and | Indoxacarb |
| B-37 | 10 | and | Lambda-cyhalothrin |
| B-38 | 10 | and | Lufenuron |
| B-39 | 10 | and | Metaflumizone |
| B-40 | 10 | and | Methomyl |
| B-41 | 10 | and | Methoprene |
| B-42 | 10 | and | Methoxyfenozide |
| B-43 | 10 | and | Nitenpyram |
| B-44 | 10 | and | Nithiazine |
| B-45 | 10 | and | Novaluron |
| B-46 | 10 | and | Oxamyl |
| B-47 | 10 | and | Pymetrozine |
| B-48 | 10 | and | Pyrethrin |
| B-49 | 10 | and | Pyridaben |
| B-50 | 10 | and | Pyridalyl |
| B-51 | 10 | and | Pyriproxyfen |
| B-52 | 10 | and | Ryanodine |
| B-53 | 10 | and | Spinetoram |
| B-54 | 10 | and | Spinosad |
| B-55 | 10 | and | Spirodiclofen |
| B-56 | 10 | and | Spiromesifen |
| B-57 | 10 | and | Tebufenozide |
| B-58 | 10 | and | Thiacloprid |
| B-59 | 10 | and | Thiamethoxam |
| B-60 | 10 | and | Thiodicarb |
| B-61 | 10 | and | Thiosultap-sodium |
| B-62 | 10 | and | Tralomethrin |
| B-63 | 10 | and | Triazamate |
| B-64 | 10 | and | Triflumuron |
| B-65 | 10 | and | Bacillus thuringiensis |
| B-66 | 10 | and | Bacillus thuringiensis |
| | | | delta-endotoxin |
| B-67 | 10 | and | NPV (e.g., Gemstar) |

The specific mixtures listed in Table B typically combine a compound of Formula 1 with the other invertebrate pest agent in the ratios specified in Table A.

Invertebrate pests are controlled in agronomic and nonagronomic applications by applying one or more compounds of this invention, typically in the form of a composition, in a biologically effective amount, to the environment of the pests, including the agronomic and/or nonagronomic locus of infestation, to the area to be protected, or directly on the pests to be controlled.

Thus the present invention comprises a method for controlling an invertebrate pest in agronomic and/or nonagronomic applications, comprising contacting the invertebrate pest or its environment with a biologically effective amount of one or more of the compounds of the invention, or with a composition comprising at least one such compound or a composition comprising at least one such compound and a biologically effective amount of at least one additional biologically active compound or agent. Examples of suitable compositions comprising a compound of the invention and a biologically effective amount of at least one additional biologically active compound or agent include granular compositions wherein the additional active compound is present on the same granule as the compound of the invention or on granules separate from those of the compound of the invention.

To achieve contact with a compound or composition of the invention to protect a field crop from invertebrate pests, the compound or composition is typically applied to the seed of the crop before planting, to the foliage (e.g., leaves, stems, flowers, fruits) of crop plants, or to the soil or other growth medium before or after the crop is planted.

One embodiment of a method of contact is by spraying. Alternatively, a granular composition comprising a compound of the invention can be applied to the plant foliage or the soil. Compounds of this invention can also be effectively delivered through plant uptake by contacting the plant with a composition comprising a compound of this invention applied as a soil drench of a liquid formulation, a granular formulation to the soil, a nursery box treatment or a dip of transplants. Of note is a composition of the present invention in the form of a soil drench liquid formulation. Also of note is a method for controlling an invertebrate pest comprising contacting the invertebrate pest or its environment with a biologically effective amount of a compound of the present invention or with a composition comprising a biologically effective amount of a compound of the present invention. Of further note is this method wherein the environment is soil and the composition is applied to the soil as a soil drench formulation. Of further note is that compounds of this invention are also effective by localized application to the locus of infestation. Other methods of contact include application of a compound or a composition of the invention by direct and residual sprays, aerial sprays, gels, seed coatings, microencapsulations, systemic uptake, baits, ear tags, boluses, foggers, fumigants, aerosols, dusts and many others. One embodiment of a method of contact is a dimensionally stable fertilizer granule, stick or tablet comprising a compound or composition of the invention. The compounds of this invention can also be impregnated into materials for fabricating invertebrate control devices (e.g., insect netting).

Compounds of this invention are also useful in seed treatments for protecting seeds from invertebrate pests. In the context of the present disclosure and claims, treating a seed means contacting the seed with a biologically effective amount of a compound of this invention, which is typically formulated as a composition of the invention. This seed treatment protects the seed from invertebrate soil pests and generally can also protect roots and other plant parts in contact with the soil of the seedling developing from the germinating seed. The seed treatment may also provide protection of foliage by translocation of the compound of this invention or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate.

One method of seed treatment is by spraying or dusting the seed with a compound of the invention (i.e. as a formulated composition) before sowing the seeds. Compositions formulated for seed treatment generally comprise a film former or adhesive agent. Therefore typically a seed coating composition of the present invention comprises a biologically effective amount of a compound of Formula 1, an N-oxide or a salt thereof, and a film former or adhesive agent. Seed can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment Progress and Prospects, 1994 BCPC Mongraph No. 57, and references listed therein.

The treated seed typically comprises a compound of the present invention in an amount from about 0.1 g to 1 kg per 100 kg of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention can be incorporated into a bait composition that is consumed by an invertebrate pest or used within a device such as a trap, bait station, and the like. Such a bait composition can be in the form of granules which comprise (a) active ingredients, namely a biologically effective amount of a compound of Formula 1, an N-oxide, or a salt thereof; (b) one or more food materials; optionally (c) an attractant, and optionally (d) one or more humectants. Of note are granules or bait compositions which comprise between about 0.001-5% active ingredients, about 40-99% food material and/or attractant; and optionally about 0.05-10% humectants, which are effective in controlling soil invertebrate pests at very low application rates, particularly at doses of active ingredient that are lethal by ingestion rather than by direct contact. Some food materials can function both as a food source and an attractant. Food materials include carbohydrates, proteins and lipids. Examples of food materials are vegetable flour, sugar, starches, animal fat, vegetable oil, yeast extracts and milk solids. Examples of attractants are odorants and flavorants, such as fruit or plant extracts, perfume, or other animal or plant component, pheromones or other agents known to attract a target invertebrate pest. Examples of humectants, i.e. moisture retaining agents, are glycols and other polyols, glycerine and sorbitol. Of note is a bait composition (and a method utilizing such a bait composition) used to control at least one invertebrate pest selected from the group consisting of ants, termites and cockroaches. A device for controlling an invertebrate pest can comprise the present bait composition and a housing adapted to receive the bait composition, wherein the housing has at least one opening sized to permit the invertebrate pest to pass through the opening so the invertebrate pest can gain access to the bait composition from a location outside the housing, and wherein the housing is further adapted to be placed in or near a locus of potential or known activity for the invertebrate pest.

The compounds of this invention can be applied without other adjuvants, but most often application will be of a formulation comprising one or more active ingredients with suitable carriers, diluents, and surfactants and possibly in combination with a food depending on the contemplated end use. One method of application involves spraying a water dispersion or refined oil solution of a compound of the present invention. Combinations with spray oils, spray oil concentrations, spreader stickers, adjuvants, other solvents, and synergists such as piperonyl butoxide often enhance compound efficacy. For nonagronomic uses such sprays can be applied from spray containers such as a can, a bottle or other container, either by means of a pump or by releasing it from a pressurized container, e.g., a pressurized aerosol spray can. Such spray compositions can take various forms, for example, sprays, mists, foams, fumes or fog. Such spray compositions thus can further comprise propellants, foaming agents, etc. as the case may be. Of note is a spray composition comprising a biologically effective amount of a compound or a composition of the present invention and a carrier. One embodiment of such a spray composition comprises a biologically effective amount of a compound or a composition of the present invention and a propellant. Representative propellants include, but are not limited to, methane, ethane, propane, butane, isobutane, butene, pentane, isopentane, neopentane, pentene, hydrofluorocarbons, chlorofluorocarbons, dimethyl ether, and mixtures of the foregoing. Of note is a spray composition (and a method utilizing such a spray composition dispensed from a spray container) used to control at least one invertebrate pest selected from the group consisting of mosquitoes, black flies, stable flies, deer flies, horse flies, wasps, yellow jackets, hornets, ticks, spiders, ants, gnats, and the like, including individually or in combinations.

Nonagronomic applications include protecting an animal, particularly a vertebrate, more particularly a homeothermic vertebrate (e.g., mammal or bird) and most particularly a mammal, from an invertebrate parasitic pest by administering a parasiticidally effective (i.e. biologically effective) amount of a compound of the invention, typically in the form of a composition formulated for veterinary use, to the animal to be protected. Therefore of note is a method for protecting an animal comprising administering to the animal a parasiticidally effective amount of a compound of the invention. As referred to in the present disclosure and claims, the terms “parasiticidal” and “parasiticidally” refers to observable effects on an invertebrate parasite pest to provide protection of an animal from the pest. Parasiticidal effects typically relate to diminishing the occurrence or activity of the target invertebrate parasitic pest. Such effects on the pest include necrosis, death, retarded growth, diminished mobility or lessened ability to remain on or in the host animal, reduced feeding and inhibition of reproduction. These effects on invertebrate parasite pests provide control (including prevention, reduction or elimination) of parasitic infestation or infection of the animal. Examples of invertebrate parasitic pests controlled by administering a parasiticidally effective amount of a compound of the invention to an animal to be protected include ectoparasites (arthropods, acarines, etc) and endoparasites (helminths, e.g., nematodes, trematodes, cestodes, acanthocephalans, etc.). In particular, the compounds of this invention are effective against ectoparasites including: flies such as Haematobia (Lyperosia) irritans (horn fly), Stomoxys calcitrans (stable fly), Simulium spp. (blackfly), Glossina spp. (tsetse flies), Hydrotaea irritans (head fly), Musca autumnalis (face fly), Musca domestica (house fly), Morellia simplex (sweat fly), Tabanus spp. (horse fly), Hypoderma bovis, Hypoderma lineatum, Lucilia sericata, Lucilia cuprina (green blowfly), Calliphora spp. (blowfly), Protophormia spp., Oestrus ovis (nasal botfly), Culicoides spp. (midges), Hippobosca equine, Gastrophilus instestinalis, Gastrophilus haemorrhoidalis and Gastrophilus naslis; lices such as Bovicola (Damalinia) bovis, Bovicola equi, Haematopinus asini, Felicola subrostratus, Heterodoxus spiniger, Lignonathus setosus and Trichodectes canis; keds such as Melophagus ovinus; mites such as Psoroptes spp., Sarcoptes scabei, Chorioptes bovis, Demodex equi, Cheyletiella spp., Notoedres cati, Trombicula spp. and Otodectes cyanotis (ear mites); ticks such as Ixodes spp., Boophilus spp., Rhipicephalus spp., Amblyomma spp., Dermacentor spp., Hyalomma spp. and Haemaphysalis spp.; and fleas such as Ctenocephalides felis (cat flea) and Ctenocephalides canis (dog flea).

Nonagronomic applications in the veterinary sector are by conventional means such as by enteral administration in the form of, for example, tablets, capsules, drinks, drenching preparations, granulates, pastes, boli, feed-through procedures, or suppositories; or by parenteral administration, such as by injection (including intramuscular, subcutaneous, intravenous, intraperitoneal), implants; by nasal administration; by topical administration, for example, in the form of immersion or dipping, spraying, washing, coating with powder, or application to a small area of the animal, and through articles such as neck collars, ear tag's, tail bands, limb bands or halters which comprise compounds or compositions of the present invention.

Typically a parasiticidal composition according to the present invention comprises a mixture of a compound of Formula 1, an N-oxide or a salt thereof, with one or more pharmaceutically or veterinarily acceptable carriers comprising excipients and auxiliaries selected with regard to the intended route of administration (e.g., oral, topical or parenteral administration such as injection) and in accordance with standard practice. In addition, a suitable carrier is selected on the basis of compatibility with the one or more active ingredients in the composition, including such considerations as stability relative to pH and moisture content. Therefore of note is a composition for protecting an animal from an invertebrate parasitic pest comprising a parasitically effective amount of a compound of the invention and at least one carrier.

For parenteral administration including intravenous, intramuscular and subcutaneous injection, a compound of the present invention can be formulated in suspension, solution or emulsion in oily or aqueous vehicles, and may contain adjuncts such as suspending, stabilizing and/or dispersing agents. Pharmaceutical compositions for injection include aqueous solutions of water-soluble forms of active ingredients (e.g., a salt of an active compound), preferably in physiologically compatible buffers containing other excipients or auxiliaries as are known in the art of pharmaceutical formulation.

For oral administration including solutions (the most readily available form for absorption), emulsions, suspensions, pastes, gels, capsules, tablets, boluses powders, granules, rumen-retention and feed/water/lick blocks, a compound of the present invention can be formulated with binders/fillers known in the art to be suitable for oral administration compositions, such as sugars (e.g., lactose, sucrose, mannitol, sorbitol), starch (e.g., maize starch, wheat starch, rice starch, potato starch), cellulose and derivatives (e.g., methylcellulose, carboxymethylcellulose, ethylhydroxycellulose), protein derivatives (e.g., zein, gelatin), and synthetic polymers (e.g., polyvinyl alcohol, polyvinylpyrrolidone). If desired, lubricants (e.g., magnesium stearate), disintegrating agents (e.g., cross-linked polyvinylpyrrolidinone, agar, alginic acid) and dyes or pigments can be added. Pastes and gels often also contain adhesives (e.g., acacia, alginic acid, bentonite, cellulose, xanthangum, colloidal magnesium aluminum silicate) to aid in keeping the composition in contact with the oral cavity and not being easily ejected.

If the parasiticidal compositions are in the form of feed concentrates, the carrier is typically selected from high-performance feed, feed cereals or protein concentrates. Such feed concentrate-containing compositions can, in addition to the parasiticidal active ingredients, comprise additives promoting animal health or growth, improving quality of meat from animals for slaughter or otherwise useful to animal husbandry. These additives can include, for example, vitamins, antibiotics, chemotherapeutics, bacteriostats, fungistats, coccidiostats and hormones.

Compounds of the present invention may have favorable pharmacokinetic and pharmacodynamic properties providing systemic availability from oral administration and ingestion. Therefore after ingestion by the animal to be protected, parasiticidally effective concentrations of compounds of the invention in the bloodstream protect the treated animal from blood-sucking pests such as fleas, ticks and lice. Therefore of note is a composition for protecting an animal from an invertebrate parasite pest in a form for oral administration (i.e. comprising, in addition to a parasiticidally effective amount of a compound of the invention, one or more carriers selected from binders and fillers suitable for oral administration and feed concentrate carriers).

Formulations for topical administration are typically in the form of a powder, cream, suspension, spray, emulsion, foam, paste, aerosol, ointment, salve or gel. More typically a topical formulation is a water-soluble solution, which can be in the form of a concentrate that is diluted before use. Parasiticidal compositions suitable for topical administration typically comprise a compound of the present invention and one or more topically suitable carriers. In applications of a parasiticidal composition topically to the exterior of an animal as a line or spot (i.e. “spot-on” treatment), the active ingredient is expected to migrate over the surface of the active to cover most or all of its external surface area. As a result, the treated animal is particularly protected from invertebrate pests that feed off the epidermis of the animal such as ticks, fleas and lice. Therefore formulations for topical localized administration often comprise at least one organic solvent to facilitate transport of the active ingredient over the skin and/or penetration into the epidermis of the animal. Solvents commonly used as carriers in such formulations include propylene glycol, paraffins, aromatics, esters such as isopropyl myristate, glycol ethers, and alcohols such as ethanol and n-propanol.

The rate of application required for effective control (i.e. “biologically effective amount”) will depend on such factors as the species of invertebrate to be controlled, the pest's life cycle, life stage, its size, location, time of year, host crop or animal, feeding behavior, mating behavior, ambient moisture, temperature, and the like. Under normal circumstances, application rates of about 0.01 to 2 kg of active ingredients per hectare are sufficient to control pests in agronomic ecosystems, but as little as 0.0001 kg/hectare may be sufficient or as much as 8 kg/hectare may be required. For nonagronomic applications, effective use rates will range from about 1.0 to 50 mg/square meter but as little as 0.1 mg/square meter may be sufficient or as much as 150 mg/square meter may be required. One skilled in the art can easily determine the biologically effective amount necessary for the desired level of invertebrate pest control.

In general for veterinary use, a compound of Formula 1, an N-oxide or a salt thereof, is administered in a parasiticidally effective amount to an animal to be protected from invertebrate parasite pests. A parasiticidally effective amount is the amount of active ingredient needed to achieve an observable effect diminishing the occurrence or activity of the target invertebrate parasite pest. One skilled in the art will appreciate that the parasitically effective dose can vary for the various compounds and compositions of the present invention, the desired parasitical effect and duration, the target invertebrate pest species, the animal to be protected, the mode of application and the like, and the amount needed to achieve a particular result can be determined through simple experimentation.

For oral administration to homeothermic animals, the daily dosage of a compound of the present invention typically ranges from about 0.01 mg/kg to about 100 mg/kg, more typically from about 0.5 mg/kg to about 100 mg/kg, of animal body weight. For topical (e.g., dermal) administration, dips and sprays typically contain from about 0.5 ppm to about 5000 ppm, more typically from about 1 ppm to about 3000 ppm, of a compound of the present invention.

The following TESTS demonstrate the control efficacy of compounds of this invention on specific pests. “Control efficacy” represents inhibition of invertebrate pest development (including mortality) that causes significantly reduced feeding. The pest control protection afforded by the compounds is not limited, however, to these species. See Index Tables A-B for compound descriptions. The following abbreviations are used in the Index Tables which follow: Me is methyl, i-Pr is isopropyl, n-Pr is normal propyl. t-Bu is tert-butyl, Ph is phenyl and CF₃ means trifluoromethyl. The abbreviation “Ex.” stands for “Example” and is followed by a number indicating in which example the compound is prepared.

INDEX TABLE A

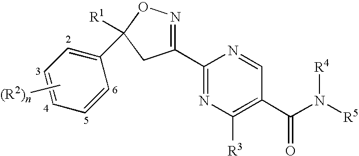

| Com- | | | | | | m.p. |
|---|---|---|---|---|---|---|
| pound | R¹ | (R²)ₙ | R³ | R⁴ | R⁵ | (° C.) |
| 1 | CF₃ | 3-Cl, 5-Cl | Me | H | CH₂CF₃ | * |
| (Ex. 1) | | | | | | |
| 2 | CF₃ | 3-Cl, 5-Cl | Me | H | CH₂-2-pyridinyl | * |
| (Ex. 2) | | | | | | |
| 3 | CF₃ | 3-Cl, 5-Cl | Me | H | Me | * |
| 4 | CF₃ | 3-Cl, 5-Cl | Ph | H | CH₂-2-pyridinyl | * |
| 5 | CF₃ | 3-Cl, 5-Cl | i-Pr | H | CH₂-2-pyridinyl | * |
| 6 | CF₃ | 3-Cl, 5-Cl | t-Bu | H | CH₂-2-pyridinyl | * |
| 7 | CF₃ | 3-Cl, 5-Cl | CF₃ | H | CH₂-2-pyridinyl | * |
| 8 | CF₃ | 3-Cl, 5-Cl | n-Pr | H | CH₂-2-pyridinyl | * |
| 9 | CF₃ | 3-Cl, 5-Cl | CF₃ | H | Me | * |
| 10 | CF₃ | 3-Cl, 5-Cl | CF₃ | H | CH₂CF₃ | * |

*See Index Table C for ¹H NMR data

INDEX TABLE B

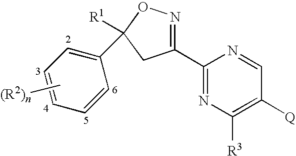

| Com- | | | | | m.p. |
|---|---|---|---|---|---|
| pound | R¹ | R² | R³ | Q | (° C.) |
| 11 | CF₃ | 3-Cl, 5-Cl | t-Bu | 1H-1,2,4-triazol-1-yl | * |
| 12 | CF₃ | 3-Cl, 5-Cl | CF₃ | 1H-1,2,4-triazol-1-yl | * |
| (Ex. 3) | | | | | |
| 13 | CF₃ | 3-Cl, 5-Cl | 2,4-di-F-Ph | 1H-1,2,4-triazol-1-yl | * |
| 14 | CF₃ | 3-Cl, 5-Cl | Me | 1H-1,2,4-triazol-1-yl | * |

*See Index Table C for ¹H NMR data

INDEX TABLE C

| Compd. | ¹H NMR Data (CDCl₃ solution |
|---|---|
| No. | unless indicated otherwise)^{a} |
| 1 | δ 8.76 (s, 1H), 7.51 (m, 2H), 7.4 (m, 1H), 6.3 (br s, 1H), |
| | 4.23 (q, 2H), 4.2 (m, 2H), 3.8 (d, 1H), 2.72 (s, 3H). |
| 2 | δ 8.9 (s, 1H), 8.6 (d, 1H), 7.8 (m, 1H), 7.5 (m, 2H), 7.4 |
| | (s, 1H), 7.2 (m, 1H), 7.1 (m, 1H), 4.8 (d, 2H), 4.25 (d, 1H), |
| | 3.88 (d, 1H), 2.77 (s, 3H). |
| 3 | δ 8.72 (s, 1H), 7.52 (m, 2H), 7.4 (s, 1H), 5.9 (br s, 1H), |
| | 4.25 (d, 1H), 3.8 (d, 1H), 3.06 (d, 3H), 2.72 (s, 3H). |
| 4 | δ 9.4 (m, 1H), 9.02 (s, 1H), 8.5 (m, 1H), 7.76 (m, 3H), |
| | 7.74 (m, 2H), 7.6 (m, 1H), 7.5 (m, 2H), 7.3 (m, 1H), 7.2 |
| | (m, 1H), 4.5 (m, 2H), 4.4 (d, 1H), 4.3 (d, 1H). |
| 5 | δ 8.82 (s, 1H), 8.6 (br s, 1H), 7.7 (m, 1H), 7.6 (m, 2H), |
| | 7.5 (m, 1H), 7.4 (m, 1H), 7.3 (d, 1H), 7.25 (m, 1H), 4.8 1H), |
| | (d, 2H), 4.2 (d, 1H), 3.8 (d, 1H), 3.6 (q, 2H), 1.33 (d, 6H). |
| 6 | δ 8.69 (s, 1H), 8.5 (br s, 1H), 7.8 (m, 1H), 7.53 (m, 2H), |
| | 7.5 (s, 1H), 7.4 (s, 1H), 7.35 (m, 1H), 7.25 (m, 1H), 7.2 |
| | (m, 1H), 4.75 (d, 2H), 4.2 (d, 1H), 3.8 (d, 1H), 1.43 (s, 9H). |
| 7 | δ 9.18 (s, 1H), 8.5 (br s, 1H), 7.8 (t, 1H), 7.65 (s, 1H), |
| | 7.52 (m, 2H), 7.5 (s, 1H), 7.3 (d, 1H), 7.25 (m, 1H), 4.78 |
| | (d, 2H), 4.3 (d, 1H), 3.85 (d, 1H). |
| 8 | δ 8.85 (s, 1H), 8.5 (s, 1H), 7.7 (m, 1H), 7.54 (m, 2H), 7.5 |
| | (s, 1H), 7.2 (d, 1H), 7.0 (m, 2H), 4.78 (m, 2H), 4.2 (d, 1H), |
| | 3.8 (d, 1H), 3.0 (m, 2H), 1.8 (m, 2H), 0.99 (m, 3H). |
| 9 | δ 9.09 (s, 1H), 7.51 (s, 2H), 7.44 (s, 1H), 6.0 (br s, 1H), |
| | 4.3 (d, 1H), 3.9 (d, 1H), 3.08 (s, 3H). |
| 10 | δ 9.11 (s, 1H), 7.51 (s, 2H), 7.45 (s, 1H), 6.4 (br s, 1H), |
| | 4.3 (d, 1H), 4.2 (m, 2H), 3.9(d, 1H). |
| 11 | δ 8.55 (s, 1H), 8.3 (s, 1H), 8.2 (s, 1H), 7.6 (m, 2H), |
| | 7.4 (s, 1H), 4.3 (d, 1H), 3.9 (d, 1H), 1.23 (s, 9H). |
| 12 | δ 9.24 (s, 1H), 8.5 (s, 1H), 8.25 (s, 1H), 7.54 (m, 2H), |
| | 7.53 (m, 1H), 4.3 (d, 1H), 3.95 (d, 1H). |
| 13 | δ 9.1 (s, 1H), 8.11 (s, 1H), 8.09 (s, 1H), 7.8 (m, 1H), |
| | 7.6 (m, 2H), 7.4 (s, 1H), 7.2 (m, 1H), 6.8 (m, 1H), 4.4 (d, |
| | 1H), 3.8 (d, 1H). |
| 14 | δ 8.78 (s, 1H), 8.4 (s, 1H), 8.2 (s, 1H), 7.61 (m, 2H), |
| | 7.4 (s, 1H), 4.4 (d, 1H), 3.8 (d, 1H), 2.64 (s, 3H). |

^{a1}H NMR data are in ppm downfield from tetramethylsilane. Couplings are designated by (s)-singlet, (d)-doublet, (t)-triplet, (q)-quartet, (m)-multiplet, (br s)-broad singlet.

### BIOLOGICAL EXAMPLES OF THE INVENTION

### Test A

For evaluating control of diamondback moth (Plutella xylostella) the test unit consisted of a small open container with a 12-14-day-old radish plant inside. This was pre-infested (i.e. infested before spraying with experimental compounds) with 10-15 neonate larvae on a piece of insect diet by use of a core sampler to remove a plug from a sheet of hardened insect diet having many larvae growing on it and transfer the plug containing larvae and diet to the test unit. The larvae moved onto the test plant as the diet plug dried out.

Test compounds were formulated using a solution containing 10% acetone, 90% water and 300 ppm X-77™ Spreader Lo-Foam Formula non-ionic surfactant containing alkylarylpolyoxyethylene, free fatty acids, glycols and isopropanol (Loveland Industries, Inc. Greeley, Colo., USA). The formulated compounds were applied in 1 mL of liquid through a SUJ2 atomizer nozzle with ⅛ JJ custom body (Spraying Systems Co. Wheaton, Ill., USA) positioned 1.27 cm (0.5 inches) above the top of each test unit. All experimental compounds in these tests were sprayed at 250 ppm replicated three times. After spraying of the formulated test compound, each test unit was allowed to dry for 1 h and then a black, screened cap was placed on top. The test units were held for 6 days in a growth chamber at 25° C. and 70% relative humidity. Plant feeding damage was then visually assessed based on foliage consumed.

Of the compounds of Formula 1 tested the following provided very good to excellent levels of plant protection (20% or less feeding damage or 80% or more mortality): 1, 2, 3, 5, 6, 7, 8, 9, 10, 12 and 14.

### Test B

For evaluating control of the Western Flower Thrip (Frankliniellla occidentalis) through contact and/or systemic means, the test unit consisted of a small open container with a 5-7-day old Longio bean plant inside.

Test compounds were formulated and sprayed at 250 ppm and replicated three times as described for Test A. After spraying, the test units were allowed to dry for 1 hour, then 22-27 adult thrips were added to the unit and then a black, screened cap was placed on top. The test units were held for 7 days at 25° C. and 45-55% relative humidity. Each test unit was then visually assessed for insect mortality

Of the compounds of Formula 1 tested, the following resulted in very good to excellent levels of pest control (at least 80% mortality): 2, 7 and 10.

### Test C

For evaluating control of fall armyworm (Spodoptera frugiperda) the test unit consisted of a small open container with a 4-5-day-old corn (maize) plant inside. This was infested (using a core sampler) with 10-15 1-day-old larvae on a piece of insect diet. Test compounds were then formulated and sprayed at 250 ppm as described for Test A and replicated three times. After spraying, the test units were maintained in a growth chamber and then visually rated as described for Test A.

Of the compounds of Formula 1 tested, the following provided very good to excellent levels of plant protection (20% or less feeding damage or 80% or more mortality): 5, 7 and 10.

### Test D

For evaluating control of potato leafhopper (Empoasca fabae Harris) through contact and/or systemic means, the test unit consisted of a small open container with a 5-6-day old Longio bean plant (primary leaves emerged) inside. White sand was added to the top of the soil and one of the primary leaves was excised prior to application. Test compounds were formulated and sprayed at 50 ppm and replicated three times as described for Test A. After spraying, the test units were allowed to dry for 1 h before they were infested with 5 potato leafhoppers (18 to 21 day old adults). A black, screened cap was placed on the top of the cylinder. The test units were held for 6 days in a growth chamber at 19-21° C. and 50-70% relative humidity. Each test unit was then visually assessed for insect mortality.

Of the compounds of Formula 1 tested, the following resulted in very good to excellent levels of pest control (at least 80% mortality): 7 and 10.

What is claimed is:

1. A compound of Formula 1, an N-oxide, or a salt thereof, 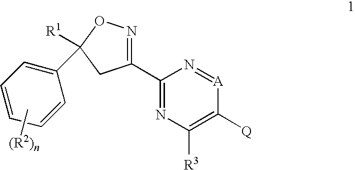
wherein:
A is selected from the group consisting of CR³ and N;
R¹ is C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁶;
each R² is independently H, halogen, C₁-C₆ alkyl, C₁-C₆ haloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkoxycarbonyl, —CN or —NO₂;
each R³ is independently H, halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, —CN, —NO₂, or —CR⁹═NOR¹⁰; or a phenyl ring or a pyridinyl ring, each ring optionally substituted with one to three substituents independently selected from R⁸;
Q is a 5- or 6-membered saturated or unsaturated heterocyclic ring optionally substituted with one or more substituents independently selected from halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, —CN, —NO₂, —N(R¹¹)R¹², —C(W)N(R¹³)R¹⁴, —C(O)OR¹⁵ and
R¹⁶; or
Q is —C(═W)NR⁴R⁵;
each R⁴, R¹¹ and R¹³ is independently H, C₁-C₆ alkyl, C₂-C₆ alkenyl, C₂-C₆ alkynyl, C₃-C₆ cycloalkyl, C₄-C₇ alkylcycloalkyl, C₄-C₇ cycloalkylalkyl, C₂-C₇ alkylcarbonyl or C₂-C₇ alkoxycarbonyl;
each R⁵, R¹², R¹⁴ and R¹⁵ is independently H; or C₁-C₆ alkyl, C₂-C₆ alkenyl, C₂-C₆ alkynyl, C₃-C₆ cycloalkyl, C₄-C₇ alkylcycloalkyl or C₄-C₇ cycloalkylalkyl, each optionally substituted with one or more substituents independently selected from R⁷;
each R⁶ is independently halogen, C₁-C₆ alkyl, C₁-C₆ alkoxy, C₁-C₆ alkylthio, C₁-C₆ alkylsulfinyl, C₁-C₆ alkylsulfonyl, —CN or —NO₂;
each R⁷ is independently halogen, C₁-C₆ alkyl, C₁-C₆ alkoxy, C₁-C₆ alkylthio, C₁-C₆ alkylsulfinyl, C₁-C₆ alkylsulfonyl, C₂-C₇ alkylcarbonyl, C₂-C₇ alkoxycarbonyl, —CN or —NO₂; or Q¹;
each Q¹ is independently a phenyl ring or a 5- or 6-membered saturated or unsaturated heterocyclic ring, each ring optionally substituted with one or more substituents independently selected from halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, —CN, —NO₂, phenyl and pyridinyl;
each R⁸ is independently halogen, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkoxycarbonyl, —CN or —NO₂;
each R⁹ is independently H, NH₂, C₁-C₄ alkyl or C₁-C₄ haloalkyl;
each R¹⁰ is independently H, C₁-C₄ alkyl or C₁-C₄ haloalkyl;
each R¹⁶ is independently a phenyl ring or a pyridinyl ring, each ring optionally substituted with one or more substituents independently selected from R¹⁷;
each R¹⁷ is independently halogen, C₁-C₆ alkyl, C₁-C₆ haloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, C₁-C₆ alkylamino, C₂-C₆ dialkylamino, C₂-C₄ alkylcarbonyl, C₂-C₄ alkoxycarbonyl, C₂-C₇ alkylaminocarbonyl, C₃-C₇ dialkylaminocarbonyl, —OH, —NH₂, —COOH, —CN or —NO₂;
W is O or S; and
n is 1, 2, 3, 4 or 5.

2. A compound of claim 1 wherein
R¹ is C₁-C₃ alkyl optionally substituted with one or more substituents independently selected from R⁶;
each R² is independently H, halogen, C₁-C₆ haloalkyl, C₁-C₆ haloalkoxy or —CN;
each R³ is independently H, halogen, C₁-C₆ alkyl, C₁-C₆ haloalkyl, C₃-C₆ cycloalkyl, C₁-C₆ alkoxy, C₁-C₆ haloalkoxy, —CN, —NO₂ or —CR⁹═NOR¹⁰; or a phenyl ring or a pyridinyl ring, each ring optionally substituted with one to three substituents independently selected from R⁸;
Q is a pyridinyl ring, a pyrimidinyl ring, a triazinyl ring, a pyrazolyl ring, a triazolyl ring, a tetrazolyl ring, an imidazolyl ring, an oxazolyl ring, an isoxazolyl ring, a thiazolyl ring or an isothiazolyl ring, each ring optionally substituted with one or more substituents independently selected from the group consisting of halogen, C₁-C₆ alkyl, C₁-C₆ haloalkyl, C₃-C₆ cycloalkyl, C₃-C₆ halocycloalkyl, C₁-C₆ alkoxy, C₁-C₆ haloalkoxy, C₁-C₆ alkylthio, C₁-C₆ haloalkylthio, C₁-C₆ alkylsulfinyl, C₁-C₆ haloalkylsulfinyl, C₁-C₆ alkylsulfonyl, C₁-C₆ haloalkylsulfonyl, —CN, —NO₂, —N(R¹¹)R¹², —C(W)N(R¹³)R¹⁴, —C(O)OR¹⁵ and R¹⁶; or
Q is C(═W)NR⁴R⁵;
each R⁴, R¹¹ and R¹³ is independently H, C₁-C₆ alkyl, C₂-C₇ alkylcarbonyl or C₂-C₇ alkoxycarbonyl;
each R⁵, R¹², R¹⁴ and R¹⁵ is independently H; or C₁-C₄ alkyl, C₂-C₄ alkenyl, C₂-C₄ alkynyl, C₃-C₄ cycloalkyl, C₄-C₇ alkylcycloalkyl or C₄-C₇ cycloalkylalkyl, each optionally substituted with one or more substituents independently selected from R⁷; and
each R⁷ is independently halogen, C₁-C₄ alkyl, C₁-C₄ alkoxy, C₁-C₄ alkylthio, C₁-C₄ alkylsulfinyl, C₁-C₄ alkylsulfonyl, C₂-C₄ alkylcarbonyl, C₂-C₄ alkoxycarbonyl, —CN, —NO₂ or Q¹.

3. A compound of claim 2 wherein
R¹ is C₁-C₃ alkyl independently substituted with halogen;
each R² is independently H, halogen, CF₃, OCF₃ or —CN;
each R³ is independently H, C₁-C₄ alkyl, C₁-C₄ haloalkyl, cyclopropyl, C₁-C₄ alkoxy, —CN or —NO₂; or a phenyl ring optionally substituted with one to three substituents independently selected from R⁸;
Q is a pyrazolyl ring, a triazolyl ring, a tetrazolyl ring or an imidazolyl ring, each ring attached to the remainder of Formula 1 through nitrogen and optionally substituted with one or more substituents independently selected from the group consisting of halogen, C₁-C₄ alkyl, C₁-C₄ haloalkyl, C₁-C₄ alkoxy, C₁-C₄ haloalkoxy, —CN and NH₂; or
Q is —C(═W)NR⁴R⁵;
R⁴ is H;
R⁵ is C₁-C₄ alkyl optionally substituted with one of more substituents independently selected from R⁷;
each R⁷ is independently halogen or Q¹; and
Q¹ is a phenyl ring, a pyridinyl ring or a thiazolyl ring, each ring optionally substituted with one or more substituents independently selected from the group consisting of halogen, C₁-C₃ alkyl, C₁-C₃ haloalkyl, —CN, phenyl and pyridinyl.

4. A compound of claim 3 wherein
R¹ is CF₃; and
R⁵ is CH₂CF₃ or CH₂-2-pyridinyl.

5. A compound of claim 1 that is selected from the group consisting of:
2-[5-(3,5-dichlorophenyl)-4,5-dihydro-5-(trifluoromethyl)-3-isoxazolyl]-N-(2-pyridinylmethyl)-4-(trifluoromethyl)-5-pyrimidinecarboxamide;
2-[5-(3,5-dichlorophenyl)-4,5-dihydro-5-(trifluoromethyl)-3-isoxazolyl]-N-(2,2,2-trifluoroethyl)-4-(trifluoromethyl)-5-pyrimidinecarboxamide;
2-[5-(3,5-dichlorophenyl)-4,5-dihydro-5-(trifluoromethyl)-3-isoxazolyl]-4-methyl-N-(2,2,2-trifluoroethyl)-5-pyrimidinecarboxamide; and
2-[5-(3,5-dichlorophenyl)-4,5-dihydro-5-(trifluoromethyl)-3-isoxazolyl]-4-methyl-N-(2-pyridinylmethyl)-5-pyrimidinecarboxamide.

6. A composition comprising a compound of claim 1 and at least one additional component selected from the group consisting of a surfactant, a solid diluent and a liquid diluent, said composition optionally further comprising at least one additional biologically active compound or agent.

7. A composition for controlling an invertebrate pest comprising a biologically effective amount of a compound of claim 1 and at least one additional component selected from the group consisting of a surfactant, a solid diluent and a liquid diluent, said composition optionally further comprising a biologically effective amount of at least one additional biologically active compound or agent.

8. The composition of claim 7 wherein at least one additional biologically active compound or agent is selected from insecticides of the group consisting of macrocyclic lactones, neonicotinoids, octopamine receptor ligands, ryanodine receptor ligands, ecdysone agonists, sodium channel modulators, chitin synthesis inhibitors, nereisotoxin analogs, mitochondrial electron transport inhibitors, cholinesterase inhibitors, cyclodiene insecticides, molting inhibitors, GABA-regulated chloride channel blockers, juvenile hormone mimics, lipid biosynthesis inhibitors and biological agents including nucleopolyhedro virus, a member of Bacillus thuringiensis, an encapsulated delta-endotoxin of Bacillus thuringiensis; and a naturally occurring or a genetically modified viral insecticide.

9. The composition of claim 8 wherein at least one additional biologically active compound or agent is selected from the group consisting of abamectin, acephate, acetamiprid, acetoprole, aldicarb, amidoflumet, amitraz, avermectin, azadirachtin, azinphos-methyl, bifenthrin, bifenazate, bistrifluoron, buprofezin, carbofuran, cartap, chinomethionat, chlorfenapyr, chlorfluazuron, chlorantraniliprole, chlorpyrifos, chlorpyrifosmethyl, chlorobenzilate, chromafenozide, clothianidin, cyflumetofen, cyfluthrin, beta-cyfluthrin, cyhalothrin, gamma-cyhalothrin, lambda-cyhalothrin, cyhexatin, cypermethrin, cyromazine, deltamethrin, diafenthiuron, diazinon, dicofol, dieldrin, dienochlor, diflubenzuron, dimefluthrin, dimethoate, dinotefuran, diofenolan, emamectin, endosulfan, esfenvalerate, ethiprole, etoxazole, fenamiphos, fenazaquin, fenbutatin oxide, fenothiocarb, fenoxycarb, fenpropathrin, fenpyroximate, fenvalerate, fipronil, flonicamid, flubendiamide, flucythrinate, tau-fluvalinate, flufenerim, flufenoxuron, fonophos, halofenozide, hexaflumuron, hexythiazox, hydramethylnon, imicyafos, imidacloprid, indoxacarb, isofenphos, lufenuron, malathion, metaflumizone, metaldehyde, methamidophos, methidathion, methomyl, methoprene, methoxychlor, methoxyfenozide, metofluthrin, monocrotophos, nitenpyram, nithiazine, novaluron, noviflumuron, oxamyl, parathion, parathion-methyl, permethrin, phorate, phosalone, phosmet, phosphamidon, pirimicarb, profenofos, profluthrin, propargite, protrifenbute, pymetrozine, pyrafluprole, pyrethrin, pyridaben, pyridalyl, pyrifluquinazon, pyriprole, pyriproxyfen, rotenone, ryanodine, spinetoram, spinosad, spiridiclofen, spiromesifen, spirotetramat, sulprofos, tebufenozide, tebufenpyrad, teflubenzuron, tefluthrin, terbufos, tetrachlorvinphos, thiacloprid, thiamethoxam, thiodicarb, thiosultap-sodium, tolfenpyrad, tralomethrin, triazamate, trichlorfon, triflumuron, Bacillus thuringiensis subsp. aizawai, Bacillus thuringiensis subsp. kurstaki, nucleopolyhedro virus, an encapsulated delta-endotoxin of Bacillus thuringiensis, baculovirus, entomopathogenic bacteria, entomopathogenic virus and entomopathogenic fungi.

10. The composition of claim 9 wherein at least one additional biologically active compound or agent is selected from the group consisting of abamectin, acetamiprid, amitraz, avermectin, azadirachtin, bifenthrin, buprofezin, cartap, chlorantraniliprole, chlorfenapyr, chlorpyrifos, clothianidin, cyfluthrin, beta-cyfluthrin, cyhalothrin, lambda-cyhalothrin, cypermethrin, cyromazine, deltamethrin, dieldrin, dinotefuran, diofenolan, emamectin, endosulfan, esfenvalerate, ethiprole, fenothiocarb, fenoxycarb, fenvalerate, fipronil, flonicamid, flubendiamide, flufenoxuron, hexaflumuron, hydramethylnon, imidacloprid, indoxacarb, lufenuron, metaflumizone, methomyl, methoprene, methoxyfenozide, nitenpyram, nithiazine, novaluron, oxamyl, pymetrozine, pyrethrin, pyridaben, pyridalyl, pyriproxyfen, ryanodine, spinetoram, spinosad, spirodiclofen, spiromesifen, tebufenozide, thiacloprid, thiamethoxam, thiodicarb, thiosultap-sodium, tralomethrin, triazamate, triflumuron, Bacillus thuringiensis subsp. aizawai, Bacillus thuringiensis subsp. kurstaki, nucleopolyhedro virus and an encapsulated delta-endotoxin of Bacillus thuringiensis.

11. The composition of claim 7 in the form of a soil drench liquid formulation.

12. A spray composition for controlling an invertebrate pest, comprising:
(a) a biologically effective amount of the compound of claim 1 or the composition of claim 7; and
(b) a propellant.

13. A bait composition for controlling an invertebrate pest, comprising:
(a) a biologically effective amount of the compound of claim 1 or the composition of claim 7;
(b) one or more food materials;
(c) optionally an attractant; and
(d) optionally a humectant.

14. A trap device for controlling an invertebrate pest, comprising:
(a) the bait composition of claim 13; and
(b) a housing adapted to receive the bait composition, wherein the housing has at least one opening sized to permit the invertebrate pest to pass through the opening so the invertebrate pest can gain access to the bait composition from a location outside the housing, and wherein the housing is further adapted to be placed in or near a locus of potential or known activity for the invertebrate pest.

15. A method for controlling an invertebrate pest comprising contacting the invertebrate pest or its environment with a biologically effective amount of a compound of claim 1.

16. A method for controlling an invertebrate pest comprising contacting the invertebrate pest or its environment with a composition of claim 7.

17. The method of claim 16 wherein the environment is soil and the composition is applied to the soil as a soil drench formulation.

18. A method for controlling a cockroach, an ant or a termite, comprising contacting a cockroach, an ant, or a termite with the bait composition in a trap device of claim 14.

19. A method for controlling a mosquito, a black fly, a stable, fly, a deer fly, a horse fly, a wasp, a yellow jacket, a hornet, a tick, a spider, an ant, or a gnat, comprising contacting a mosquito, a black fly, a stable, fly, a deer fly, a horse fly, a wasp, a yellow jacket, a hornet, a tick, a spider, an ant, or a gnat with the spray composition of claim 12 dispensed from a spray container.

20. A method for protecting a seed from an invertebrate pest comprising contacting the seed with a biologically effective amount of a compound of claim 1.

21. The method of claim 20 wherein the seed is coated with the compound of claim 1 formulated as a composition comprising a film former or adhesive agent.
